(19) United States
(12) Patent Application Publication
(10) Pub. No.: US 2014/0350081 A1
(43) Pub. Date: Nov. 27, 2014

(54) INSECT G-COUPLED RECEPTORS USEFUL AS TARGETS FOR INSECTICIDES AND COMPOUNDS AND REAGENTS IDENTIFIED USING THE SAME

(71) Applicant: Purdue Research Foundation, West Lafayette, IN (US)

(72) Inventors: Catherine A. Hill, Lafayettte, IN (US); Val J. Watts, West Lafayette, IN (US)

(21) Appl. No.: 14/356,019

(22) PCT Filed: Nov. 5, 2012
(86) PCT No.: PCT/US2012/063585

§ 371 Date: May 2, 2014

Related U.S. Application Data
(60) Provisional application No. 61/555,645, filed on Nov. 4, 2011.

(51) Int. Cl. A01N 43/90; C12Q 1/68

(52) U.S. Cl. CPC A01N 43/90; C12Q 1/68
USPC 514/44 A; 506/9; 435/6.12

(57) ABSTRACT

An approach to identify and evaluate potential insecticide targets using publicly available genome (DNA) sequence information for arthropod disease vector is provided. The utility of this approach is demonstrated by first determining the molecular and pharmacological properties of two different dopamine (neurotransmitter) receptors identified in the genome of the yellow fever- and dengue-transmitting mosquito, Aedes aegypti. Next, different chemistries were tested for their ability to interact with one of these dopamine receptors in a chemical compound screen, and “hit” compounds were identified. Finally, it is shown that some of these chemistries, are selective for the mosquito over the human dopamine receptor and that these chemistries caused significant mortality in mosquito larvae 24 hours after exposure.

### PRIORITY CLAIM

This application claims the benefit of U.S. Provisional Patent Application No. 61/555,645, filed on Nov. 4, 2011, and incorporated herein by reference in its entirety.

### STATEMENT OF GOVERNMENTAL RIGHTS

This invention was made with government support under grant contract number 09-CRIS-2-2037/IND011474 awarded by the United States Department of Agriculture/National Institute of Food and Agriculture; and grant contract no. prime award W81XWH-10-1-0085, sub-award no. PU201596 awarded by the Department of Defense/Telemedicine and Advanced Technology Research Center. The United States Government has certain rights in the invention.

### FIELD OF THE INVENTION

Aspects of the invention related to identifying G-coupled receptors in insects such as mosquitoes and the design and use of screens that employ these proteins to identify efficacious insecticides.

### BACKGROUND

Many neglected tropical infectious diseases affecting humans are transmitted by arthropods such as mosquitoes and ticks. New mode-of-action chemistries are urgently sought to enhance vector management practices in countries where arthropod-borne diseases are endemic, especially where vector populations have acquired widespread resistance to insecticides.

### SUMMARY

Some aspects of the invention include a method of controlling a population of invertebrates. The method comprises contacting an invertebrate with a compound that includes at least one of the group consisting of asenapine, amperozide, cis-(Z)-flupenthixol, benztropine, methiothepin, loxapine, mianserin, clomipramine, amperozide, butaclamol, clozapine, doxepin, and SCH23390 amitriptyline, chlorpromazine chlorprothixene. In some more particular embodiments, the invertebrate is an insect. In some more particular embodiments, the compound includes at least one of the group consisting of asenapine, amperozide, and cis-(z)-flupenthixol. In some more particular embodiments, the compound includes at least one of the group consisting of benztropine, methiothepin, loxapine, chlorprothixene, mianserin, and clomipramine. In some more particular embodiments, the insect is a mosquito. In some more particular embodiments, the compound includes at least one of the group consisting of asenapine, amperozide, cis-(Z)-flupenthixol, chlorpormazine, amitriptyline, and doxepin. In some more particular embodiments, the insect is a termite. In some more particular embodiments, the compound includes at least one of the group consisting of asenapine, cis-(Z)-flupenthixol, amperozide, amitriptyline, and chlorpromazine. In some more particular embodiments, the insect is a cockroach. in some more particular embodiments, the compound includes at least one of the group consisting of amitriptyline, chlorpormazine, cis-(Z)-flupenthixol, asenapine, and amperozide. In some more particular embodiments, the invertebrate is an arthropod. In some more particular embodiments, the invertebrate is a tick. In some more particular embodiments, the compound includes at least one of asenapine, chlorpromazine, and amperozide.

Some aspects of the invention include a method of controlling a population of invertebrates. The method comprises contacting an invertebrate with a compound that includes at least one of the group consisting of amitriptyline hydrochloride, (±)-butaclamol hydrochloride, clozapine, doxepin hydrochloride, cis-(Z)-flupenthixol dihydrochloride, methiothepin maleate, mianserin hydrochloride, niclosamide, piceatannol, and resveratrol. In some more particular embodiments, the invertebrate is any one of an arthropod, an insect, a mosquito, a termite, a cockroach, and a tick.

Some aspects of the invention include a method of controlling a population of invertebrates. The method comprises contacting an invertebrate with a compound that includes at least one of the group of chemical scaffolds consisting of dibenzocycloheptane derivatives, phenothiazine derivatives, thioxanthene derivatives, butyrophenone derivatives, diphenyl amine-containing compounds, quinazoline derivatives, benzodiazoxide derivatives, indole derivatives, piperazinylpyrazolopyrimidines, aryldiaminopryimidine, and hexahydrothienopyridines. In some more particular embodiments, the invertebrate is any one of an arthropod, an insect, a mosquito, a termite, a cockroach, and a tick.

Some aspects of the invention include a method of controlling a population of invertebrates. The method comprises contacting an invertebrate with a compound that includes at least 3 conjugated or non-conjugated rings, wherein said rings are independently selected from the group consisting of aromatic, non-aromatic, heterocyclic and non-heterocyclic rings, and wherein heterocyclic rings may independently includes at least one of the following atoms: C, O, N, or S and wherein each ring may be independently, substituted or unsubstituted with at least one of the following groups or atoms including H, amines, imines, ketones, aldehydes, alcohols, thiols, aromatic rings, alkanes, alkenes, alkynes, halogens and the like. In some more particular embodiments, the invertebrate is any one of an arthropod, an insect, a mosquito, a termite, a cockroach, and a tick.

Some aspects of the invention include a method of controlling a population of insects. The method includes contacting an insect with at least polynucleotide that interferes with the expression of a gene having at least 90 percent homology to SEQ ID. NO. 1 or SEQ ID. NO. 3. In some more particular embodiments, the polynucleotide interferes with the expression of at gene that includes or that hybridizes under stringent conditions to SEQ ID. NO. 1 or SEQ ID. NO. 3.

Some aspects of the invention include a method of screening. The method includes expressing a polynucleotide having at least 90 or 95 percent homology to SEQ ID. NO. 1 or SEQ ID. NO. 3, and contacting cells that express said polynucleotide with at least one exogenous compound, wherein said polynucleotide is not expressed in its native host. In some more particular embodiments, the polynucleotide has at least 90 or 95 percent identity to SEQ ID. NO. 1 or SEQ ID. NO. 3. In some more particular embodiments, the polynucleotide is SEQ ID. NO. 1 or SEQ ID. NO. 3.

Some aspects of the invention include a method of screening. The method includes an isolated polypeptide having at least 90 or 95 percent homology to SEQ ID. NO. 2 or SEQ ID. NO. 4; with at least one exogenous compound and detecting a interaction between said polypeptide and the at least one compound. In some more particular embodiments, the polypeptide has at least 90 or 95 percent identity to SEQ ID. NO. 2 or SEQ ID. NO. 4. In some more particular embodiments, the polypeptide is SEQ ID. NO. 2 or SEQ ID. NO. 4.

### SEQUENCE LISTING

SEQ ID NO. 1. Gene Aedes aegypti dopamine receptor AaDOP1 Gene sequence.

SEQ. ID NO. 2. Aedes aegypti Dopamine Receptor AaDOP1 conceptual amino acid sequences.

SEQ. ID NO. 3. AADOP2 gene sequence.

SEQ. ID NO. 4. AADOP2 conceptual amino acid sequence.

SEQ ID NO. 5. Gene Ixodes scapularis dopamine receptor IsDOP1 Gene sequence.

SEQ. ID NO. 6. Ixodes scapularis Dopamine Receptor IsDOP1 conceptual amino acid sequences.

SEQ. ID NO. 7. IsDOP2 gene sequence.

SEQ. ID NO. 8. IsDOP2 conceptual amino acid sequence.

### BRIEF DESCRIPTION OF THE FIGURES

FIG. 1A. Drug discovery and development pipeline for new insecticidal chemistries. The illustration shows critical steps involved with the “genome-to-lead” (described in this manuscript) and “lead-to-product” phases. Abbreviations: (EPA) Environmental Protection Agency; (FDA) Food and Drug Administration; (SAR) structure-activity relationship study. The intended administration route of a particular chemistry dictates the federal agency that will receive the registration package.

FIG. 1B: Expanded details of the “hit-to-lead” phase including those pursued in this study.

FIG. 2. Neighbor-joining sequence analysis of Aedes aegypti AaDOP1 and AaDOP2 and representative biogenic amine receptors. The deduced amino acid sequences for the mosquito dopamine receptors AaDOP1 and AaDOP2 and additional receptors for dopamine, muscarinic acetylcholine, octopamine, serotonin, and tyramine from Drosophila melanogaster and Apis mellifera, as well as the human D₁-like and D₂-like dopamine receptors were aligned for use in the analysis. Bootstrap values (100 replicates) are indicated with numbers at supported branches. The outgroup is a D. melanogaster diuretic hormone receptor, a Class B GPCR. Abbreviations: Aa=Ae. aegypti; Is=I. scapularis; Dm=D. melanogaster; Am=A. mellifera; Hs=H. sapiens. Sequences: Isdop1, D₁-like dopamine receptor (ISCW001496); Isdop2, D₁-like dopamine receptor (ISCW008775); DmD-Dop1, D₁-like dopamine receptor (P41596); DmDAMB, D₁-like dopamine receptor (DopR99B/DAMB: AAC47161), DmDD2R, D₂-like dopamine receptor (DD2R-606: AAN15955); DmDih, diuretic hormone 44 receptor 1 (NP_{—}610960.1); DmmAChR, muscarinic acetylcholine receptor (AAA28676); DmOAMB, octopamine receptor in mushroom bodies, isoform A (NP_{—}732541); DM5HT1A, serotonin receptor 1A, isoform A (NP_{—}476802); DmTyr, tyramine receptor (CG7431: NP_{—}650652); AmDOP1, D₁-like dopamine receptor (dopamine receptor, D1, NP_{—}001011595); AmDOP2, D₁-like dopamine receptor (dopamine receptor 2, NP_{—}001011567), AmDOP3, D₂-like dopamine receptor (AmDOP3, NP_{—}001014983); AmmAChR, muscarinic acetylcholine receptor (XP_{—}395760); AmOA1, octopamine receptor (oar, NP_{—}001011565); Am5HT1A, serotonin receptor (5ht-1, NP_{—}001164579); AmTyr, tyramine receptor (XP_{—}394231); HsD1, D₁-like dopamine receptor (D(1A), NP_{—}000785); HsD2,D₂-like dopamine receptor (D(2), NP_{—}000786); HsD3, D₂-like dopamine receptor (D(3), NP_{—}000787); HsD4, D₂-like dopamine receptor (D(4), NP_{—}000788); HsD5, D₁-like dopamine receptor (D(1B)/D5, NP_{—}000789).

FIG. 3. Alignment of the complete Aedes aegypti AaDOP1 and AaDOP2 amino acid sequences. Highlighted areas designate residues with shared biochemical characteristics, as designated by the ClustalW [33] output, where black shading=identical residues; dark shading=strongly similar residues; light shading=weakly similar residues. Also noted are the residues composing the N- and C-termini and the transmembrane (TM) domains I-VII.

FIG. 4A. Pharmacological characterization of the Aedes aegypti AaDOP1 and AaDOP2 receptors. Representative curve of biogenic amines measured with AaDOP.

FIG. 4B: Representative curve of biogenic amines measured with AaDOP2.

FIG. 4C. Synthetic dopamine receptor agonists of AaDOP1.

FIG. 4D. Synthetic dopamine receptor agonists of AaDOP2.

FIG. 4E. Inhibitory effect of 10 μM SCH23390 in the presence of 1 μM dopamine (n=4) shown for both mosquito dopamine receptors. ** p<0.01; *** p<0.001.

FIG. 4F. Dose-response curve of dopamine for AaDOP2 in the absence or presence of 10 μM SCH23390 used to identify an appropriate “signal window” for chemical library screening. The concentration of dopamine selected for screening (300 nM) is indicated with a box. CPS=counts per second; M=molarity.

FIG. 5. Dose-response curves for selected screen “hit” compounds that exhibited antagonistic effects on AaDOP2. Direct cAMP accumulation assays were used for dose-response assays and determination of IC₅₀ values for SCH23390 (antagonist control) and seven AaDOP2 antagonists (shown in Table 3) identified in the chemical library screen.

FIG. 6A. Toxicity of antagonist screen hits in Ae. aegypti larval bioassays. Aegypti larval bioassay showing toxicity of amitriptyline and doxepin at a single dose point (400 μM) compared to the water control; Ami=amitriptyline, Dox=Doxepin; * indicates p<0.05.

FIG. 6B: Aegypti larval bioassay involving amitriptyline in a dose response format (25 μM-400 μM).

FIG. 7A. Gel electrophoresis for non-quantitative RT-PCR assessment of transcript production for Aedes aegypti Aadop1 and Aadop2. Aadop1 amplified with primers Aadop_{—}1F/1R (224 bp amplicon).

FIG. 7B: Aadop2 amplified with primers Aadop2_Full_F/R (1,425 bp amplicon). Transcripts were detected for both dopamine receptors in each developmental stage of the mosquito and both adult sexes. As expected, no amplification products were detected in the negative control, which contained identical reagents as the other reactions but lacked an RNA template. Abbreviations: (M) DNA size marker (HyperLadder I, Bioline USA Inc., Randolph, Mass.); (E) egg; (L) larva; (P) pupa; (AF) adult female; (AM) adult male; (−) negative control. Marker: DNA HyperLadder I (Bioline USA Inc., Randolph, Mass.).

FIG. 8B: Aadop2. Exons (E) are shown with gray bars, and introns with solid black lines. Numbers above the box/line indicate the size of exon/intron in base pairs (bp), respectively. The putative transmembrane domains (1-VII) are shown with black boxes along the exons. The gene structures of Aadop1 and Aadop2 include three and two introns, respectively, which is consistent with other characterized insect dopamine receptor genes that also contain introns [42], but is in contrast with the single exon gene structures reported for the two D₁-like receptor genes in humans [74-75] and the Lyme disease tick, I. scapularis [36]. The genomic supercontigs on which Aadop1 and Aadop2 reside have not yet been linked to chromosomal positions [10], so their relative genome organization cannot yet be compared with other insects. However, in A. gambiae the predicted orthologs of Aadop1 and Aadop2 are positioned on chromosome 2R (GPRDOP1: AGAP004613) and the X chromosome (GPRDOP2: AGAP000667) [9].

FIG. 9. Alignment of transmembrane (TM) domains of Aedes aegypti AaDOP1 and AaDOP2 and other D₁-like dopamine receptors. Aligned receptor amino acid sequences include each of the two D₁-like receptors reported in Drosophila melanogaster (D-Dop1; DopR99B/DAMB) [30,31,39,43], Apis mellifera (AmDOP1; AmDOP2) [40], Ixodes scapularis (Mop1; Isdop2) [36,41], and Homo sapiens (HsD1, HsD5) [74-75]. Amino acids included in the alignment were related to the TM regions predicted for D. melanogaster [30-31]. Shaded amino acids designate residues conserved among each of the aligned TM domain sequences.

FIG. 10A. RT-PCR detection of transcripts for Aedes aegypti Aadop1 and Aadop2 in transiently-transfected HEK 293 cells. Gel electrophoresis shows PCR and RT-PCR amplification of Aedes aegypti Aadop1 A: DNA construct pcDNA3.1+/Aadop1 A: pcDNA3.1+/Aadop1 A: pcDNA3.1+/Aadop1 (John, cut and pasted from last page-didn't know where to insert).

FIG. 10B: Aadop2 using primers Aadop1_{—}1F/2R (amplicon=1058 bp) and Aadop2_FullF/FullR (1425 bp), respectively. Abbreviations: (M) DNA size marker (HyperLadder I, Bioline USA Inc., Randolph, Mass.); lanes under the heading “PCR” include controls for DNA contamination in the RNA preparation: (−) no DNA template; (+) and DNA construct pcDNA3.1+/Aadop2; (V) mRNA from cells transfected with empty vector pcDNA3.1; (C) mRNA from cells transfected with construct and pcDNA3.1+/Aadop2. Lanes under the heading “RT-PCR” show mRNA transcript detection experiments; (−) no template mRNA; (+) mRNA from adult female Ae. aegypti (non-specific amplification products were eliminated with gel purification); (V) mRNA from cells transfected with empty vector pcDNA3.1; (C) mRNA from cells transfected with construct and pcDNA3.1+/Aadop2.

FIG. 11. Response of AaDOP1 and AaDOP2 following dopamine treatment in transiently-transfected HEK cells. Significant responses to dopamine were observed for both AaDOP1 and AaDOP2, relative to basal conditions (p<0.05)

FIG. 12: Time course experiment showing toxicity of DAR antagonists to Ae. aegypti L3 larvae, also shown in Table 8. Percent larval mortality is shown on the y-axis. Chemistries were evaluated in comparison to a water-only control.

FIG. 13: Concentration response curves (CRCs; 0-100 μM) showing toxicity of four AaDOP2 antagonist chemistries to Ae. aegypti L3 larvae over 24 hours

FIG. 14 Neighbor joining analysis of amino acid sequences for the putative Ixodes scapularis dopamine receptors and selected receptors for dopamine, muscarinic acetylcholine, octopamine, serotonin, and tyramine from Drosophila melanogaster and Apis mellifera, as well as the human D1-like and D2-like dopamine receptors. Bootstrap values (100 replicates) are indicated with numbers at supported branches. The outgroup included a D. melanogaster diuretic hormone receptor, a Class B GPCR. Abbreviations: Is ¼ I. scapularis; Dm ¼ D. melanogaster; Am ¼ A. mellifera; Hs ¼ Homo sapiens. Sequences: Isdop1, putative dopamine receptor (ISCW001496); Isdop2, putative dopamine receptor (ISCW008775); DmD-Dop1, D1-like dopamine receptor (P41596); DmDAMB, D1-like dopamine receptor (DopR99B/DAMB: AAC47161), DmDD2R, D2-like dopamine receptor (DD2R-606: AAN15955); DmDih, diuretic hormone 44 receptor 1 (NP_{—}610960.1); DmmAChR, muscarinic acetylcholine receptor (AAA28676); DmOAMB, octopamine receptor in mushroom bodies, isoform A (NP_{—}732541); Dm5-HT1A, serotonin receptor 1A, isoform A (NP_{—}476802); DmTyr, tyramine receptor (CG7431: NP_{—}650652); AmDOP1, D1-like dopamine receptor (dopamine receptor, D1, NP_{—}001011595); AmDOP2, D1-like dopamine receptor (dopamine receptor 2, NP_{—}001011567), AmDOP3, D2-like dopamine receptor (AmDOP3, NP_{—}001014983); AmmAChR, muscarinic acetylcholine receptor (XP_{—}395760); AmOA1, octopamine receptor (Oar, NP_{—}001011565); Am5-HT1A, serotonin receptor (5ht-1, NP_{—}001164579); AmTYR1, tyramine receptor (XP_{—}394231); HsD1, D1-like dopamine receptor (D(1A), NP_{—}000785); HsD2, D2-like dopamine receptor (D(2), NP_{—}000786); HsD3, D2-like dopamine receptor (D(3), NP_{—}000787); HsD4, D2-like dopamine receptor (D(4), NP_{—}000788); HsD5, D1-like dopamine receptor (D(1B)/D5, NP_{—}000789).

FIG. 15 Amino acid alignment of the predicted TM domains (TM I-VII) of I. scapularis Isdop1 (11) and Isdop2 (U.) and the two D1-like receptors reported in Drosophila melanogaster (D1 ¼ D-Dop1; D2 ¼ DopR99B/DAMB) (Gotzes et al., 1994; Feng et al., 1996; Han et al., 1996), Apis mellifera (A1 ¼ AmDOP1; A2 ¼ AmDOP2) (Mustard et al., 2003), and Homo sapiens (H1, H5) (Sunahara et al., 1990; Sunahara et al., 1991). The amino acids included in the alignment of TM domains are related to those positions reported by Gotzes et al. (1994) and Feng et al. (1996) for the D1-like dopamine receptors in D. melanogaster. Also shown is a three amino acid extension of the third TM domain to illustrate the conserved “DRY” motif. Highlighted amino acids show positions conserved in each of the aligned dopamine receptors.

FIG. 16A Functional studies of the Isdop1 and Isdop2 receptors expressed in HEK 293 cells. Transiently transfected HEK cells were analyzed for cAMP accumulation under basal and drug-stimulated conditions. (A) Response to dopamine (10 μM), n ¼ 4; (B) Responses to the agonists SKF38393 (10 μM) and SKF81297 (10 μM) and to the antagonists SCH23390 (10 μM) and (+)-butaclamol (10 μM) in combination with 1 μM dopamine, n ¼ 4. Statistical analysis (A) Paired, two-tailed t-test, *p<0.05, **p<0.01;

FIG. 16B Functional studies of the Isdop1 and Isdop2 receptors expressed in HEK 293 cells. Transiently transfected HEK cells were analyzed for cAMP accumulation under basal and drug-stimulated conditions. (B) One-way ANOVA with each condition compared to the basal response followed by Dunnett's post-hoc test, *p<0.05, **p<0.01, ***p<0.001. DA ¼ dopamine.

FIG. 17A-C Functional studies in HEK cells stably expressing the CRE-Luc reporter gene in combination with either Isdop1 or Isdop2. (A) Dose-response curves of dopamine, epinephrine, or norepinephrine (1 nM-10 μM) activation of Isdop1 (representative graph shown); (B) Dose-response curves of dopamine (1 nM-30 μM), epinephrine. (C)SCH23390 (10 μM) inhibition of response to dopamine (1 μM), ** indicates p<0.01 using a paired one-sample t-test. The data represent the mean and standard error of a minimum of three independent experiments assayed in at least triplicate. Abbreviations: DA ¼ dopamine; Epi ¼ epinephrine; Nor ¼ norepinephrine; CPS ¼ counts per second.

FIG. 18 Dopamine dose-response curves for Isdop1 and Isdop2 highlighting the differences in constitutive activity and fold-stimulation in response to dopamine. The dopamine concentration used in the chemical library screen with Isdop2 is shown with an arrow (3 μM). Abbreviation: CPS=luminescence counts per second. Shown Is a representative graph of three independent experiments (average±S.E.M.).

FIG. 19A-F The activity of Isdop1 and Isdop2 were assessed using direct cAMP accumulation. Panels A and B: Dopamine concentration-response curves for Isdop1 (A) and Isdop2 (B) to establish parameters for the confirmation assays. The approximate EC90 values for dopamine subsequently used in the confirmation assays are labeled with arrows. Data shown are based on two independent experiments carried out in at least triplicate. Panels C to F: Confirmation assays of antagonist activities to compare the responses of Isdop1 and Isdop2 to clozapine (C), mianserin (D), SCH23390 (E), and methiothepin (F) identified in the chemical library (LOPAC1280) screen. Levels of cAMP accumulation were normalized to the response of dopamine alone (10 nM for Isdop1 and 30 μM for Isdop2), based on at least three independent experiments carried out in duplicate.

FIG. 20. Alignment of the amino acid sequences for the Isdop2 (Meyer et al., 2011) and AaDOP2 receptors (Meyer et al., 2012). Roman numerals (I to VII) denote the predicted seven transmembrane domains. The region between TMV and TMVI represents the highly divergent third intracellular loop. Black background indicates identical amino acids and gray shading indicates similar amino acids. The sequence alignment was generated using the Muscle-Multiple Sequence Alignment program (http://www.ebi.ac.uk/Tools/msa/muscle/) and managed with BioEdit software (Hall, 1999).

FIG. 21. Concentration response curves of select LOPAC compounds for AaDOP2 receptors. The AaDOP2 receptor was stably expressed in HEK 293-CRELuc cells for dose-response assays and determination of EC₅₀ values. A, Representative curves for dopamine, epinephrine, and norepinephrine. B, Representative curves for dopamine, dihydrexidine, SKF81297, and SKF38393.

### DESCRIPTION

For the purposes of promoting an understanding of the principles of the novel technology, reference will now be made to the preferred embodiments thereof, and specific language will be used to describe the same. It will nevertheless be understood that no limitation of the scope of the novel technology is thereby intended, such alterations, modifications, and further applications of the principles of the novel technology being contemplated as would normally occur to one skilled in the art to which the novel technology relates are within the scope of this disclosure and the claims.

Mosquitoes (Class Insecta; Order Diptera; Family Culicidae) vector multiple neglected tropical diseases (NTDs) affecting human health, including malaria, yellow-fever, dengue and filariasis. Historically, insecticides employed against arthropod disease vectors have reduced the impact of NTDs, but unfortunately, continued disease control is threatened by the widespread development of vector populations that are resistant to insecticidal chemistries [1]. This issue is further complicated by the fact that there has not been a new public health insecticide produced for vector-borne disease control for over 30 years [2]. Recently, philanthropic investment has focused attention toward the development of new drugs to control NTDs in the human population [3]. It is widely recognized that an arsenal of new vector control solutions are required in order to meet this and other public health goals regarding NTDs. Thus, the research community should aggressively pursue the discovery of new mode-of-action chemistries for mosquito control through both traditional phenotypic screening and novel target-based approaches.

Novel insecticide targets potentially exist among the arthropod G-protein coupled receptors (GPCRs). These proteins comprise a large family of membrane-bound molecules that mediate critical biological processes such as neurotransmission, vision, and hormonal regulation, among others [4-5]. GPCRs are extensively targeted for drug development in humans—approximately 40% of prescription pharmaceuticals interact with these receptors [6]—and more recently, Gamo et al. [7] reported multiple GPCR-interacting chemistries as promising anti-malarial leads. Also, the mode-of-action of amitraz, a chemistry registered for tick and insect control, is presumed to have partial agonistic activity at an octopamine sensitive GPCR [8]. More than 100 different GPCRs have been identified in the genomes of multiple insect species, including malaria- and yellow fever-transmitting mosquitoes [9-10]. These studies have provided a basis for the functional characterization of GPCRs and their prioritization as potential subjects for insecticide development.

The biogenic amine-binding GPCRs (rhodopsin-like) are integral components of the central and peripheral nervous systems of eukaryotes and include receptors that bind the neurotransmitters dopamine, histamine, octopamine, serotonin, tyramine, and acetylcholine [11]. The dopamine receptors are classified as either D₁- or D₂-like [12] based on their differential functional roles. Ligand binding to the D₁-like dopamine receptors causes Gαₛ-mediated stimulation of adenylyl cyclase (AC) production of cAMP. A reciprocal effect is observed following agonist activation of D₂-like dopamine receptors, whereby cAMP production by AC is inhibited via Gα_{i/o} proteins. Dopamine and its receptors are essential for complex behavioral mechanisms in arthropods such as locomotion [13-15], arousal [16], and olfactory learning [17-18].

The importance of dopaminergic-related functions has stimulated research to understand these processes in mosquitoes. Dopamine and serotonin have been tied to salivary gland functioning of vectors [19-20] and may have an impact on pathogen acquisition and transmission during blood feeding. Andersen et al. [21] reported that increased levels of dopamine were detected in Aedes aegypti following a blood meal that were implicated in ovarian or egg development, and in newly-emerged adults, presumably as part of the sclerotization process. Much attention has been given to the role of dopamine in the melanization pathway of mosquitoes and other insects, as well as the effect of dopamine on development, pigmentation, reproduction, immune responses to parasites, wound healing, and Wolbachia infection [22-27]. In the mosquito Culex pipiens, dose-dependent increases in cAMP were detected following treatment with dopamine and octopamine in homogenized head tissues, suggesting the presence of Gαₛ-coupled receptors that are responsive to these biogenic amines [28]. Putative D₁-like and D₂-like dopamine receptors have been identified in the genomes of the mosquitoes Ae. aegypti [9] and Anopheles gambiae [10], but research investigating their pharmacological properties is lacking. These genomic sequences provide a logical starting point to functionally characterize the receptors, which is needed to improve our comprehension of dopaminergic processes in mosquitoes. Moreover, due to their presumed significance in mosquito neurobiology, these dopamine receptors are attractive candidates to explore as new targets for chemical control.

Described herein is a “genome-to-lead” approach for insecticide discovery that uses perhaps the first reported chemical screen of a G-protein coupled receptor (GPCR) mined from a mosquito genome. A combination of molecular and pharmacological studies was used to functionally characterize two dopamine receptors (AaDOP1 and AaDOP2) from the yellow fever mosquito, Aedes aegypti. Sequence analyses indicated that these receptors are orthologous to arthropod D₁-like (Gαₛ-coupled) receptors, but share less than 55% amino acid identity in conserved domains with mammalian dopamine receptors. Heterologous expression of AaDOP1 and AaDOP2 in HEK293 cells revealed dose-dependent responses to dopamine (EC₅₀: AaDOP1=3.1±1.1 nM; AaDOP2=240±16 nM). Interestingly, only AaDOP1 exhibited sensitivity to epinephrine (EC₅₀=5.8±1.5 nM) and norepinephrine (EC₅₀=760±180 nM), while neither receptor was activated by other biogenic amines tested. Differential responses were observed between these receptors regarding their sensitivity to dopamine agonists and antagonists, level of maximal stimulation, and constitutive activity. Subsequently, a chemical library screen was implemented to discover lead chemistries active at AaDOP2. Fifty-one compounds were identified as “hits”, and follow-up validation assays confirmed the antagonistic effect of selected compounds at AaDOP2. In vitro comparison studies between AaDOP2 and the human D₁ dopamine receptor (hD₁) revealed markedly different pharmacological profiles and identified amitriptyline and doxepin as AaDOP2-selective compounds. In subsequent Ae. aegypti larval bioassays, significant mortality was observed for amitriptyline (93%) and doxepin (57%), confirming these chemistries as “leads” for insecticide discovery.

This research provides a pipeline useful for prioritization, pharmacological characterization, and expanded chemical screening of additional GPCRs in disease-vector arthropods. The differential molecular and pharmacological properties of the mosquito dopamine receptors highlight the potential for the identification of target-specific chemistries for vector-borne disease management, also reported are dopamine receptor antagonists with in vivo toxicity toward mosquitoes.

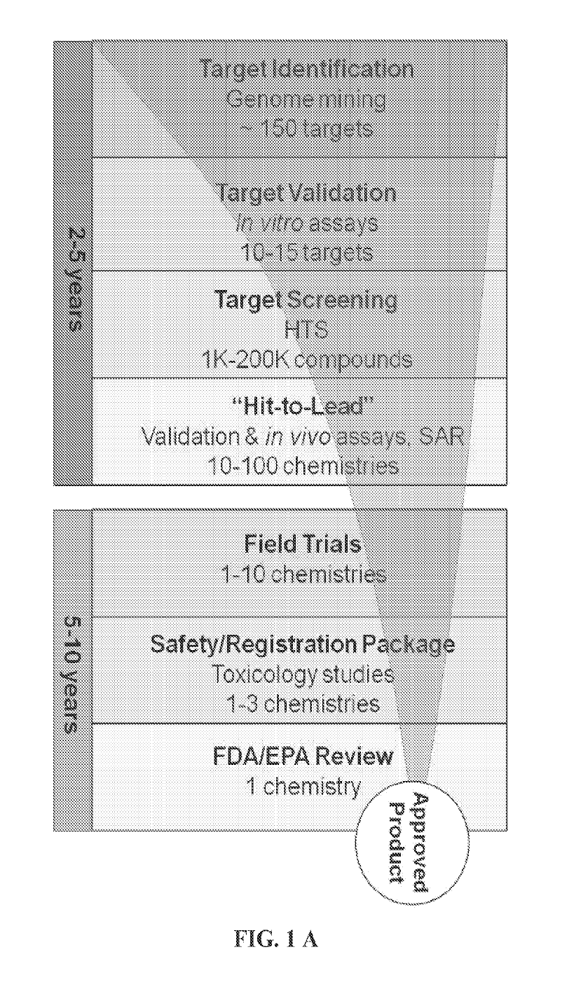

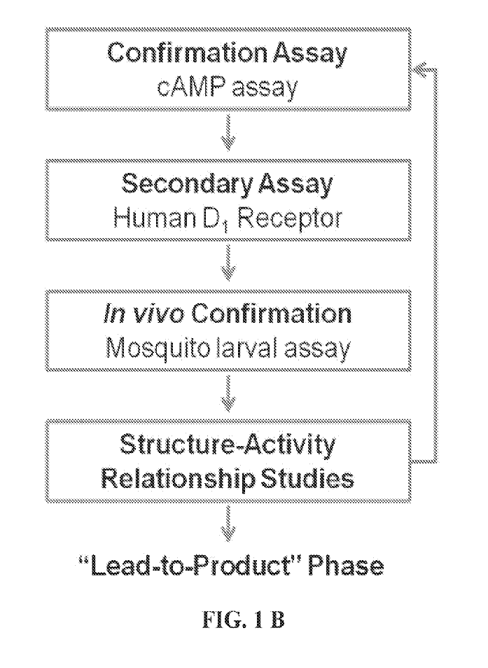

Presented herein are the results of a “proof-of-concept” study involving a “genome-to-lead” approach for developing new mode-of-action insecticides for arthropod disease vectors (FIG. 1A). This strategy involves (i) exploitation of an arthropod genome sequence for novel target identification, (ii) biochemical and pharmacological target validation, (iii) chemical library screening, and (iv) confirmation of hits and identification of candidate “leads” using secondary in vitro assays and mosquito in vivo assays. Toward these objectives, two dopamine receptors (AaDOP1 and AaDOP2) were identified in the genome of the yellow-fever mosquito, Ae. aegypti, and characterized using molecular and pharmacological methods. Next a chemical library screen in which multiple antagonistic chemistries of the AaDOP2 receptor was conducted. Finally, we employed a “hit-to-lead” approach (FIG. 1B) wherein, screen “hits” were confirmed in secondary in vitro assays and two “lead” chemistries were identified using in vivo assays that confirmed their toxicity to mosquito larvae. These results serve as an entry point for expanded chemical library screening of mosquito dopamine receptors and subsequent structure-activity relationship- and further “hit-to-lead”-studies to discover one or more candidate compounds that will enter the registration phase of product development (FIG. 1). Our pipeline will expedite the exploration of GPCRs as potential targets for chemical control in mosquitoes and other important arthropod disease vectors for which sufficient genome sequence data is available.

Materials and Methods—Aedes aegypti

### Molecular Analyses

The gene sequences for the putative dopamine receptors AaegGPRdop1 SEQ I.D. NO. 2 (AAEL003920) and AaegGPRdop2 (AAEL005834) (referred to hereafter as Aadop1 and Aadop2, respectively) in Ae. aegypti [10] were downloaded from VectorBase (http://www.vectorbase.org/index.php) [29]. Sequences of the D₁-like dopamine receptors in Drosophila melanogaster were used to identify and compare conserved structural features [30-31].

Gene expression analyses for each receptor were conducted using RNA extracted from the eggs, larvae, pupae, and adult male and female mosquitoes from the Liverpool strain of Ae. aegypti [10]. Total RNA was isolated using TRIzol Reagent (Invitrogen, Carlsbad, Calif.) and then treated with RNase-Free DNase (QIAGEN, Valencia, Calif.). The SuperScript One-Step RT-PCR kit (Invitrogen, Carlsbad, Calif.) was used to amplify receptor mRNA from approximately 150 ng total RNA per reaction using the primers and experimental conditions provided in supporting information, Table 5. RT-PCR amplification products were electrophoresed and compared by size to the DNA HyperLadder I (Bioline USA Inc., Randolph, Mass.). Products were cut from the gel and isolated with the Qiagen Gel Extraction Kit (Qiagen Valencia, Calif.). The cloning procedure was performed using the TOPO TA cloning kit (Invitrogen, Carlsbad, Calif.), according to the manufacturer's instructions. DNA sequencing was conducted at the Purdue University Genomics Core Facility. The resultant DNA sequences were used to predict full-length coding regions that were manually annotated using Artemis software (version 9) [32].

A neighbor joining sequence analysis was performed using the deduced amino acid sequences representing the mosquito dopamine receptor proteins (referred to hereafter as AaDOP1 and AaDOP2), additional representative biogenic amine receptors from the insects D. melanogaster and A. mellifera, and the human D₁- and D₂-like dopamine receptors. ClustalW 1.83 [33] was used for sequence alignments prior to tree construction in PAUP 4.0b4a [34]. The bootstrap method (100 replicates) was used to provide branch support. Alignments of amino acid sequences for determination of conserved motifs were conducted using Multalin software [35]. Conserved amino acid residues and additional protein features were predicted as described by Meyer et al. [36].

### Heterologous Expression

Functional characterization of AaDOP1 and AaDOP2 was conducted by heterologous expression in HEK293 cells (ATCC, Manassas, Va.) [36]. Expression constructs were produced by synthesis (GenScript, Piscataway, N.J.) and included the partial Kozak transcriptional recognition sequence “CACC” added directly upstream of the transcription initiation codon for each gene. Constructs were cloned into pUC57 and then subcloned into the expression vector pcDNA3.1+ (Invitrogen, Carlsbad, Calif.) by GenScript (Piscataway, N.J.). Stable cell lines co-expressing either AaDOP1 or AaDOP2 with a CRELuc reporter construct were developed to permit pharmacological studies in a 384-well format [36-37]. Briefly, cells already stably expressing the CRELuc reporter construct were transfected in a 10 cm dish with 15 μl Lipofectamine-2000 and 3 μg of pcDNA3.1+/Aadop1 or pcDNA3.1+/Aadop2. Clones were maintained as described for the wild-type HEK293 cells [36] with the addition of 2 μg/ml puromycin and 300 μg/ml Geneticin (Sigma-Aldrich, St. Louis, Mo.).

### Pharmacological Characterization

For initial functional analysis, the receptors were transiently expressed in HEK293 cells [36] and analyzed using a competitive binding assay to measure levels of cAMP accumulation [37]. Dose-response curves were generated using cells stably expressing the receptors [36-37]. The compounds used for pharmacological characterization included dopamine hydrochloride, histamine dihydrochloride, 5-hydroxytryptamine hydrochloride (serotonin), (±)-octopamine hydrochloride, and tyramine hydrochloride (Sigma-Aldrich, St. Louis, Mo.) and (−)-epinephrine bitartrate and L (−)-norepinephrine bitartrate (Research Biochemical International, Natick, Mass.). The synthetic dopamine receptor ligands tested included SKF38393 and SKF81297 (Tocris, Ellisville, Mo.), SCH23390 (Tocris, Ellisville, Mo.), and dihydrexidine (DHX). Data was collected from a minimum of three independent replicate experiments with each sample measured in triplicate. Statistical analysis of data was conducted with GraphPad Prism 5 software (GraphPad Software Inc., San Diego, Calif.).

### Screening of AaDOP2 Against the LOPAC₁₂₈₀ Library

In order to identify novel AaDOP2 receptor antagonists, a Library of Pharmacologically Active Compounds (LOPAC₁₂₈₀) was screened, using HEK-CRELuc-Aadop2 cells. These cells were cultured as described above, expanded, and cryo-preserved, to produce a uniform cell population. Briefly, cells (˜2.5×10⁷) were harvested by non-enzymatic dissociation [0.5 mM EDTA in Ca²⁺Mg²⁺ free-phosphate buffered saline (CMF-PBS)] resuspended in cell culture media, and pelleted by centrifugation for 5 min at 100×G. The pellet was resuspended in freezing media (Opti-MEM supplemented with 10% DMSO and 10% FBS) to a concentration of 5×10⁶/ml, frozen step-wise, and held in liquid N₂ until use. Cells were rapidly thawed, diluted in Opti-MEM, and 20 μl containing 25,000 cells were plated per well in 384-well plates (Nunc, Fisher Scientific, Pittsburgh, Pa.) using a BiomekFX liquid handling station (Beckman-Coulter, Brea, Calif.). The plates were incubated overnight in a humidified incubator at 37° C. and 5% CO₂.

Prior to screen initiation, a “checkerboard” analysis was conducted that included a minimum (300 nM dopamine in combination with 10 μM SCH23390) and maximum (300 nM dopamine) stimulatory condition. The data obtained were analyzed to calculate the Z-factor [38] using a modified equation that accounts for the number of replicates (NIH website: http://www.ncgc.nih.gov/guidance/section2.html#summary-calcs).

All compounds were diluted to appropriate concentrations and suspended in assay buffer (Opti-MEM supplemented with 0.02% ascorbic acid) using a BiomekFX 96-tip head. All LOPAC₁₂₈₀ compounds were screened in quadruplicate at a concentration of 10 μM, including duplicate samples on two separate assay plates in different quadrants to control for plate and automation effects. Each plate contained a dopamine response curve (14 nM-30 μM) and antagonist control wells (10 μM SCH23390 in combination with 300 nM dopamine). Following compound addition, dopamine was added to each test well at a final concentration of 300 nM, and cells were incubated for 2 hr at 37° C. in a humidified incubator. The plates were then equilibrated at 25° C. prior to the addition of Steadylite plus luminescence reagent (PerkinElmer, Shelton, Conn.). Plates were incubated on a shaker at 300 rpm for 5 min, and the luminescence signal was measured using a DTX880 multimode reader (Beckman Coulter, Brea, Calif.) with a 1 sec integration time.

Raw screen data were processed as follows: the average background luminescence (cells in the absence of dopamine or LOPAC₁₂₈₀ compound) was subtracted from the raw data. Values for the positive receptor activation control (300 nM dopamine) were averaged within each assay plate and used to establish a 100% dopamine receptor stimulation level. Similarly, the average response to SCH23390 was calculated within each assay plate to establish a baseline inhibition for antagonist chemistries. The average percent compound effect was calculated for each LOPAC chemistry in comparison to the SCH23390 antagonist control. The minimum criterion for selection of an antagonist “hit” was established as the percent inhibition equivalent to that determined for SCH23390+3 standard deviations.

### Confirmation and Secondary In Vitro Assays

Subsequent validation assays using both the AaDOP2 and the human D₁ dopamine receptor (hD₁) (Sunahara et al., 1990) were conducted for select identified chemistries using a competitive binding cAMP accumulation assay. In addition to SCH23390, these included amitriptyline hydrochloride, doxepin hydrochloride, niclosamide, clozapine, (+)-butaclamol hydrochloride, cis-(Z)-flupenthixol dihydrochloride, resveratrol, mianserin hydrochloride (Sigma, St. Louis, Mo.), piceatannol and methiothepin maleate (Tocris, Ellisville, Mo.). The drugs were suspended from dimethyl sulfoxide (DMSO) stocks in Hanks Balanced Salt Solution (HBSS) (HyClone, Logan, Utah) with 0.1% fatty acid free bovine serum albumin (BSA) and 20 mM 4-(2-hydroxyethyl)-1-piperazineethanesulfonic acid (HEPES), and serial dilutions were prepared using a Precision 2000 automated pipetting system (BioTek, Winooski, Vt.). The cAMP accumulation assay was carried out as previously described [36-37] with minor modifications to permit processing of a larger number of samples in a semi-automated fashion. Briefly, AaDOP2- or hD₁-expressing cells were harvested using Hank's based non-enzymatic cell dissociation reagent (Invitrogen, Carlsbad, Calif.), triturated in equal parts Dulbecco's modified eagle medium (DMEM) (Invitrogen, Carlsbad, Calif.) dissociation reagent, centrifuged 5 min at 100×G, and resuspended in HBSS supplemented with 0.1% BSA and 20 mM HEPES. Cells were seeded (50,000 cells in 40 μl) in clear 96-well plates and incubated at 37° C. with 5% CO₂ for 1 hr. The cAMP accumulation assay was carried out in HBSS supplemented with final concentrations of 0.1% BSA, 20 mM HEPES, 0.5 mM 3-isobutyl-1-methylxanthine (IBMX), and 0.02% ascorbic acid in a final volume of 50 μl. The selected compounds were added to the wells in duplicate, followed by addition of dopamine (final concentration 3 μM for AaDOP2 and 100 nM for hD₁). Plates were incubated at room temperature for 1 hr, and the assay was terminated by addition of 25 μl of 9% ice-cold trichloroacetic acid (TCA). Cell lysates were incubated on ice for at least 1 hr prior to quantifying cAMP accumulation as previously described [36-37].

In Vivo Ae. aegypti Bioassays

Single dose-point and dose response in vivo mosquito bioassays were used to assess the toxicity of selected AaDop2 receptor antagonists identified in the chemical screen. Larvae of Ae. aegypti (Liverpool strain) were reared under standard laboratory conditions on a 12 hr day/night cycle at 75% RH and 28° C., and bioassays were conducted at room temperature (22-24° C.). Larvae were transferred from standard rearing trays into six-well tissue culture plates (Corning, Inc. Corning, N.Y.) using a small plastic pipette. Ten L4-stage larvae were included per well, each containing five ml of de-ionized water and an assigned drug concentration. Controls were conducted similarly but lacked a drug treatment. Bioassays employed a double-blind experimental design, and larval mortality was scored 24 hr following administration of drugs. Single dose-point assays were conducted using 400 μM drug and included three biological replicates each consisting of 100 larvae. Dose-response assays were conducted using five doses (400, 200, 100, 50 and 25 μM. Five technical replicates, each including 10 larvae, were performed per dose, and the assay was repeated three times. Statistical analysis included one sample t-tests (single-point assays) and determination of the LC₅₀ and LC₉₀ values (dose-response assays) and were conducted with GraphPad Prism 5 software (GraphPad Software Inc., San Diego, Calif.).

### Results

### Molecular Analyses

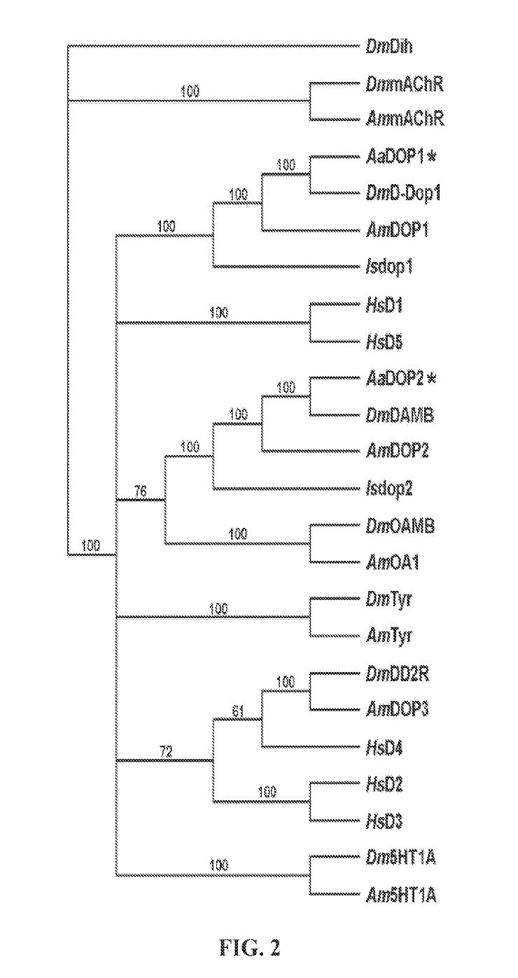

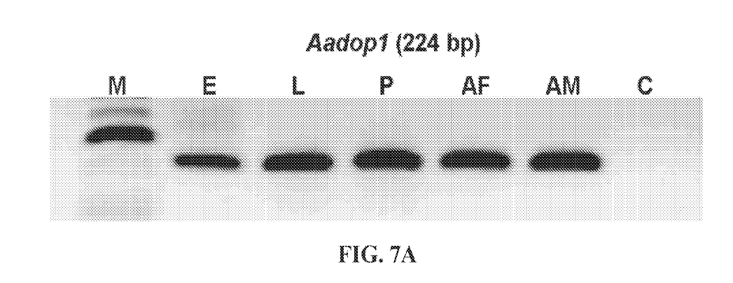

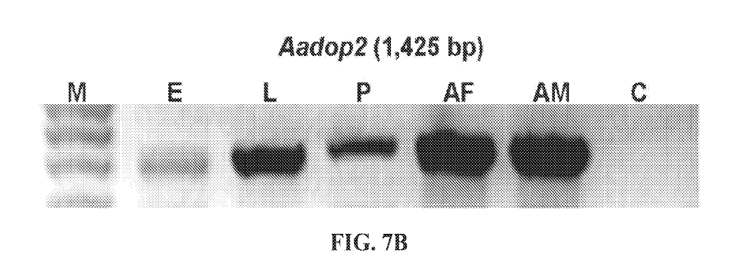

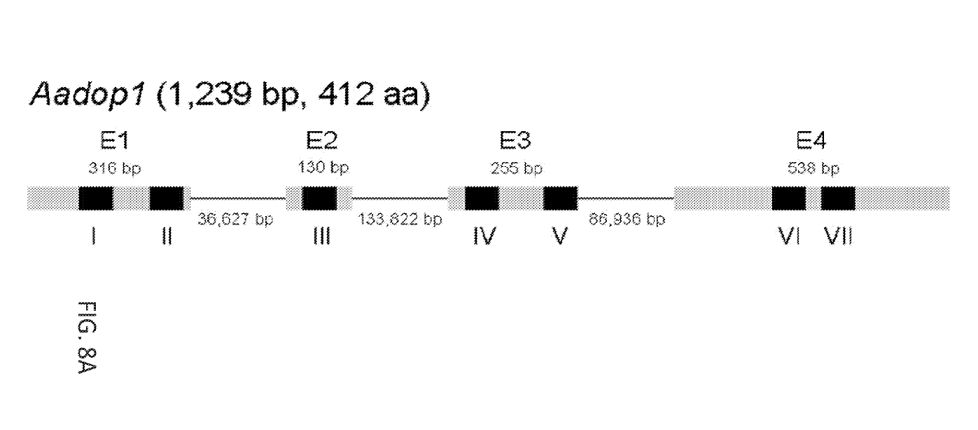

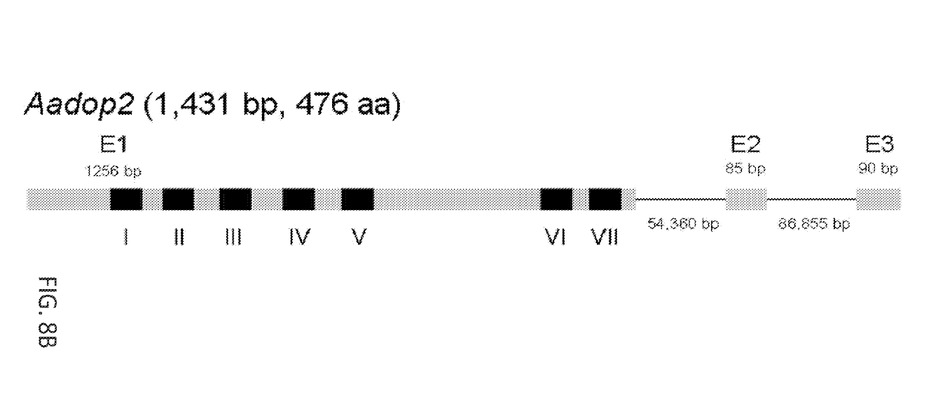

mRNA transcripts for Aadop1 and Aadop2 were detected by RT-PCR from the eggs, larvae, pupae, and adult male and female Ae. aegypti (FIGS. 7A-7B). DNA sequencing of RT-PCR products confirmed the splice junctions at each intron/exon boundary for both receptor genes. Using a combination of evidence from the RT-PCR data, the genome sequence, and related sequences in D. melanogaster, it was possible to predict the gene structure and complete coding regions of Aadop1 (Genbank accession: JN043502) and Aadop2 (Genbank accession: JN043503) FIGS. 8A-8B). A neighbor-joining sequence analysis was conducted to assess the relationships of AaDOP1 and AaDOP2 with other representative biogenic amine receptors (FIG. 2). AaDOP1 was included in a Glade (bootstrap=100) containing the presumably orthologous D₁-like dopamine receptors D-Dop1 of D. melanogaster [30,39], DOP1 of A. mellifera [40], and Mop 1 of I. scapularis [36,41]. AaDOP2 clustered with two presumably orthologous insect D₁-like dopamine receptors (INDRs) [42], DopR99B (DAMB) of D. melanogaster [31,43] and DOP2 of A. mellifera [40], as well as Isdop2 of I. scapularis [36]. The INDR-like and Isdop2 sequences were also joined together in a larger Glade (bootstrap=76) containing the octopamine receptors OAMB of D. melanogaster [44] and OCT1 [45] of A. mellifera, consistent with Mustard et al. [40]. The human D₁-like dopamine receptors formed a separate Glade (bootstrap=100) distinct from the arthropod dopamine receptors.

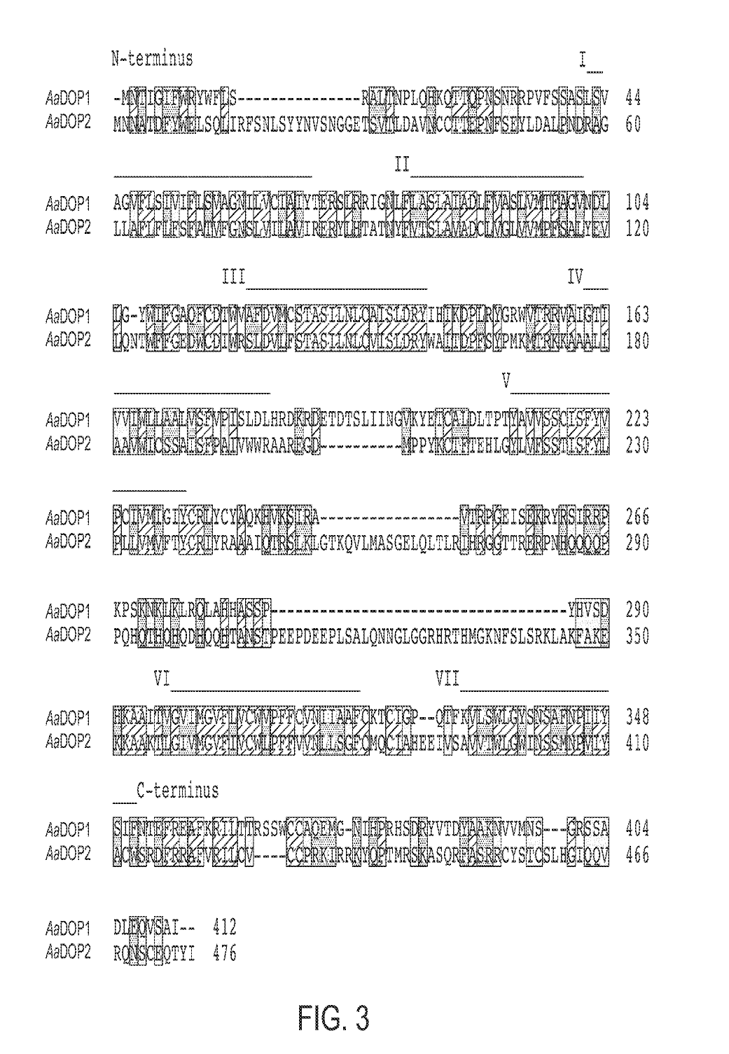

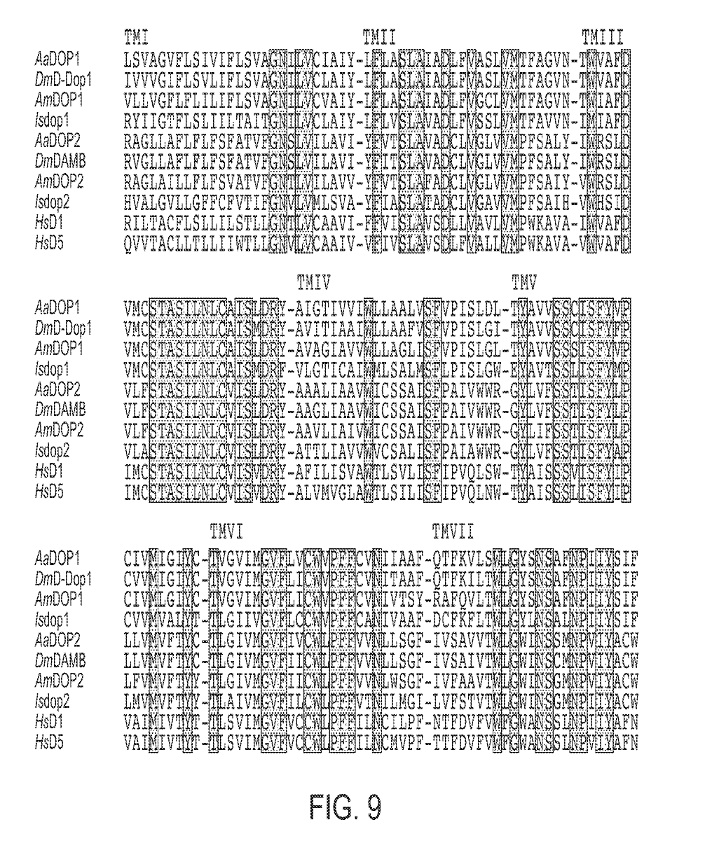

The deduced amino acid sequences of AaDOP1 and AaDOP2 were analyzed to identify conserved structural features typically associated with biogenic amine-binding GPCRs (supporting information, Table 6), as well as unique regions that could be potentially exploited for development of mosquito-specific chemistries. Conserved features included sites predicted for ligand binding, protein stability, G-protein coupling, and post-translational modification. Alignments of the full-length AaDOP1 and AaDOP2 amino acid sequences (FIG. 3) indicated that these sequences were divergent in the presumed N- and C-termini and the intracellular and extracellular loops, and the TM domains were moderately conserved (47% amino acid identity). A substantial difference was observed in the composition and relative size of the third intracellular loop that was much larger in AaDOP2 (115 amino acids) than in AaDOP1 (62 amino acids). Importantly, only a modest level of similarity was observed between the mosquito and human D₁-like dopamine receptors, which shared between 47-54% amino acid identities among the TM domains, which typically represent the most conserved regions of GPCRs (supporting information, Table 7). Moreover, comparison of the predicted TM domains from multiple invertebrate and vertebrate D₁-like dopamine receptors showed that only 34% (58/172) of the amino acids were shared among all species included in the alignment (FIG. 9). The highest level of sequence similarity to the TM domains of AaDOP1 and AaDOP2 was found in their predicted D. melanogaster orthologs, D-Dop1 (88% identity) (supporting information, Table 7) and DopR99B (97% identity), respectively.

### Heterologous Expression and Pharmacological Characterization

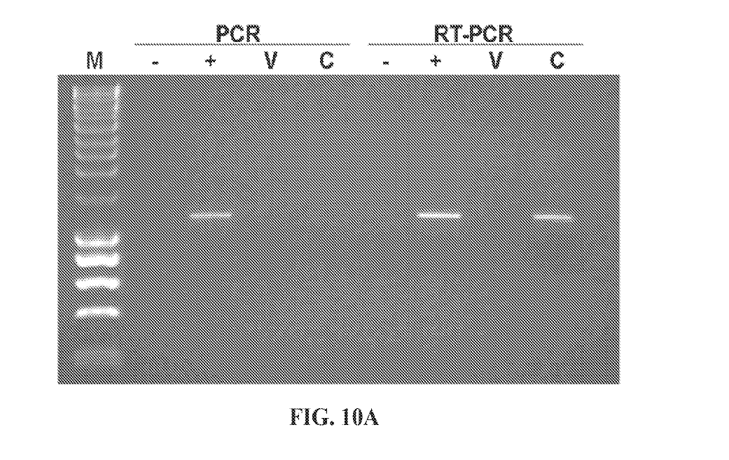

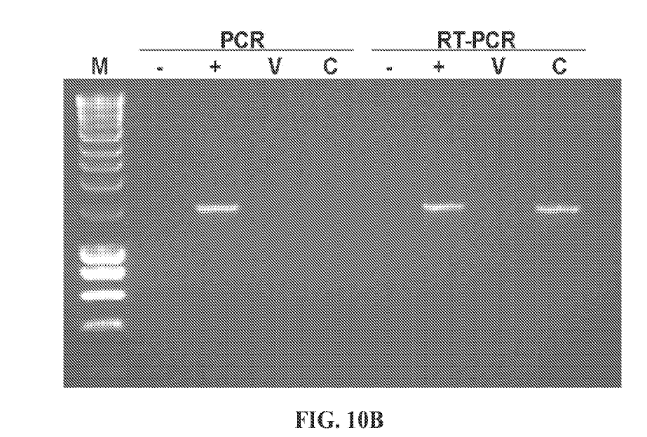

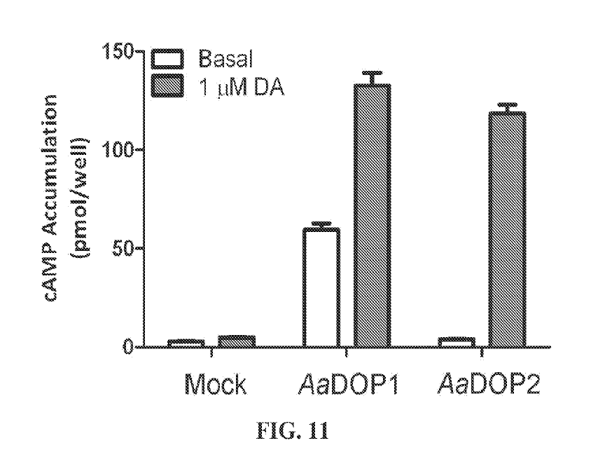

In order to study the function of the putative dopamine receptors AaDOP1-IEOID No. 3 and AaDOP2-IEOID No. 4, each receptor was expressed in HEK293 cells. Production of the mosquito receptor transcripts in transiently-transfected cells was first verified using RT-PCR (supporting information, FIGS. 10A-10B). Increases of intracellular cAMP were detected in cells transiently expressing either AaDOP1 [2.7±0.6 fold (n=3)] or AaDOP2 [48±14 fold (n=3)] in response to a single dose of dopamine (10 μM) (supporting information, FIG. 11). No significant increase in cAMP was observed in the mock transfected cells (empty pcDNA3.1+ vector). For cells transiently expressing AaDOP1, relatively high levels of constitutive activity were observed (17.6±2.4 fold greater than in mock transfected cells) as compared to AaDOP2 (1.83±0.93 fold greater than in mock transfected cells).

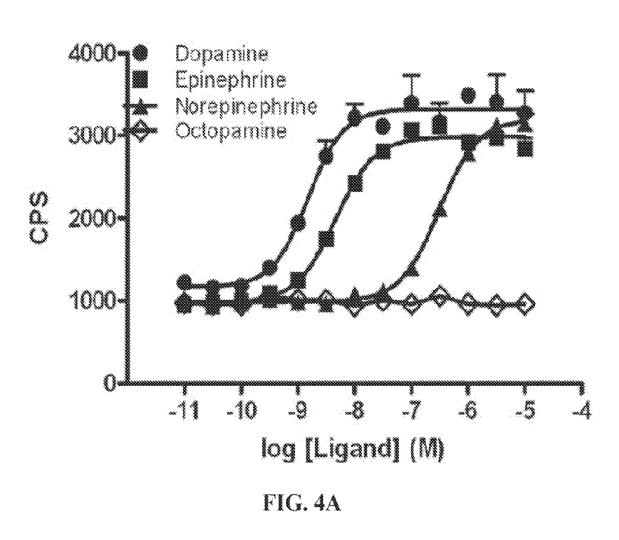

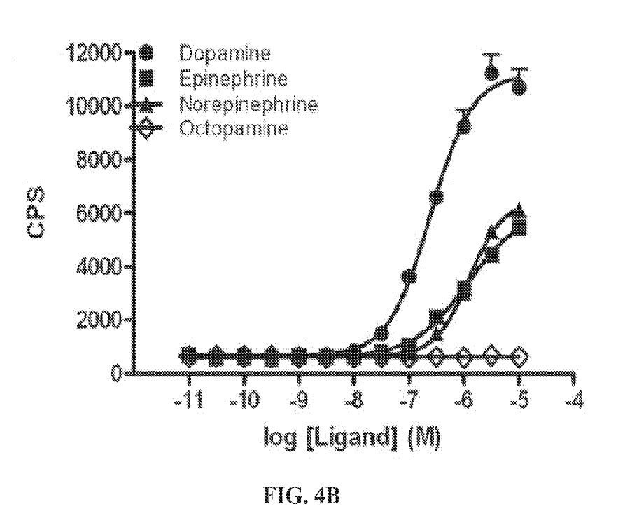

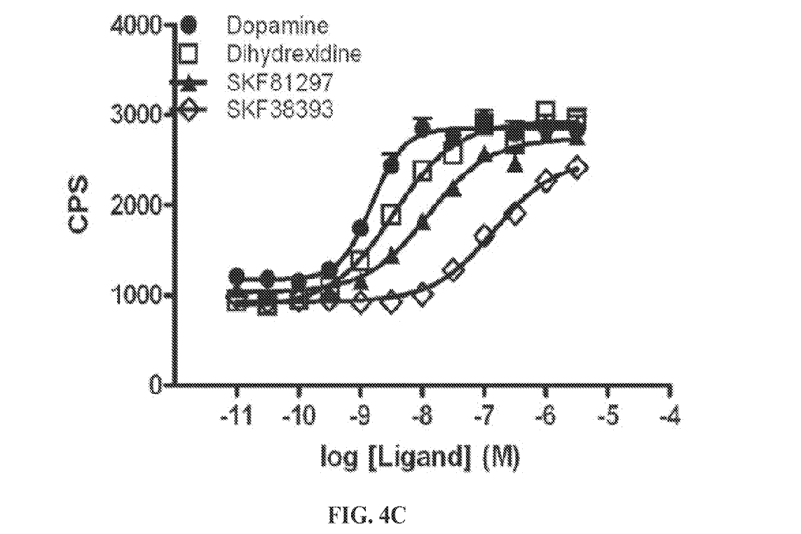

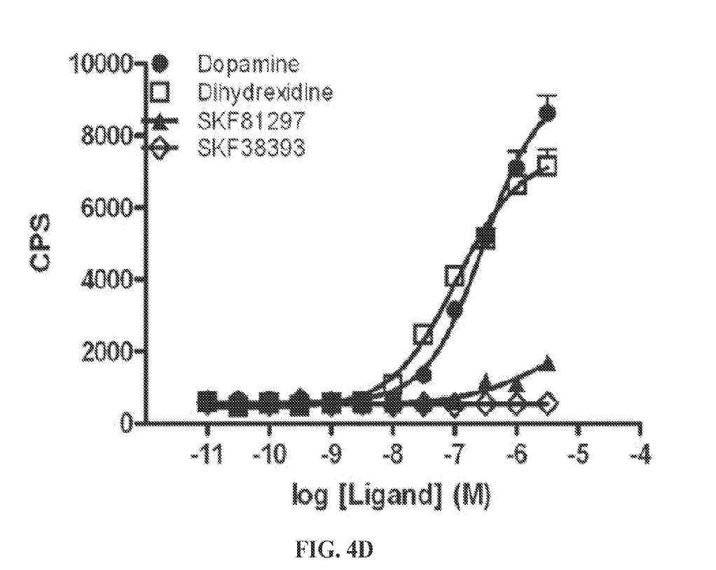

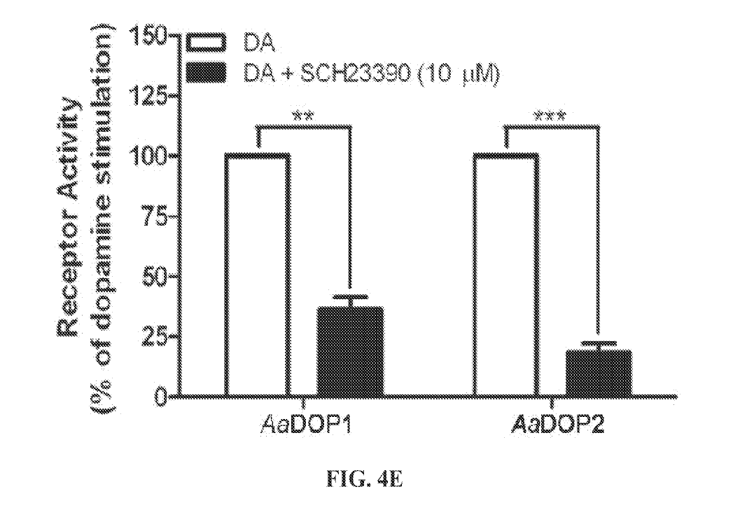

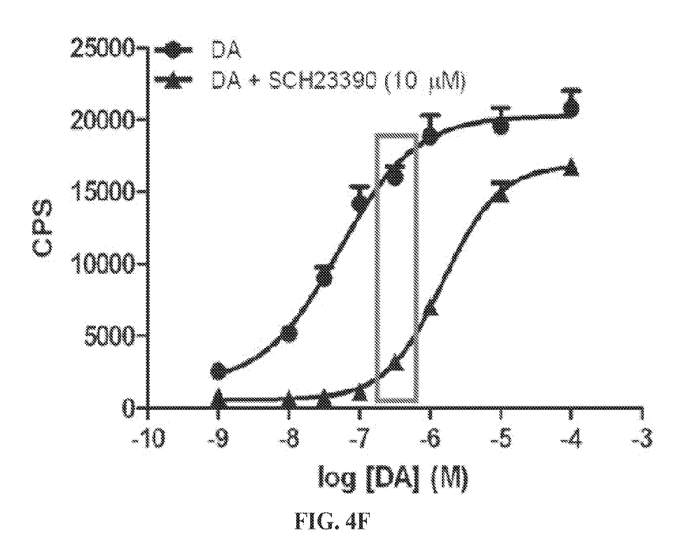

Subsequently, dose-response curves were generated using cells stably expressing either AaDOP1 or AaDOP2 for seven different biogenic amines (FIG. 4; Table 1). Again, dopamine stimulated both receptors, with EC₅₀ values determined at 3.1±1.1 nM and 240±16.0 nM for AaDOP1 and AaDOP2, respectively (FIGS. 4A-B; Table 1). In addition, we observed activation of the AaDOP1 receptor by epinephrine (EC₅₀=5.8±1.5 nM) and norepinephrine (EC₅₀=760±180 nM) (Table 1). Conversely, no significant stimulation was observed for the AaDOP2 receptor by epinephrine or norepinephrine (Table 1). Neither receptor was stimulated by histamine, octopamine, serotonin, or tyramine (EC₅₀≧10 uM). The effects of known synthetic dopamine receptor agonists were also investigated (FIGS. 4B-C; Table 1). Considerable stimulation was observed for AaDOP1 with the agonists listed in their rank order of potency: DHX>SKF81297>SKF38393. In contrast, of the synthetic agonists tested here, only treatment with DHX resulted in significant dose-dependent activation of AaDOP2. The addition of the D₁ dopamine receptor antagonist SCH23390 (10 μM) robustly inhibited the dopamine-mediated stimulation of both AaDOP1 and AaDOP2 (FIG. 4E).

### Screening of Aadop2 Against the LOPAC₁₂₈₀ Library

The AaDOP2 receptor for an antagonist screen of the LOPAC₁₂₈₀ library because of its low constitutive activity and strong dopamine response compared to background (approximately 10-fold) (FIGS. 4A-D) was selected. Using dose-response studies, it was determined that 300 nM dopamine alone and in combination with 10 μM SCH23390 created a suitable “signal window” for identification of AaDOP2 antagonists (FIG. 4F). A “checkerboard analysis” using these conditions and assuming four replicates in the screen generated a Z-factor of 0.5±0.1 (n=3), indicating that the assay was suitable for antagonist screening.

The criterion for “hit” detection was established relative to the control antagonist (SCH23390 response+3 standard deviations), such that only those compounds that inhibited the dopamine response by at least 81% were considered (Table 2). Based on this, the screen identified 51 potential antagonists of the AaDOP2 receptor (additional screen results provided in Table 8). These compounds were partitioned into seven different classes based on their known biochemical interactions with mammalian molecular targets that included dopamine receptor antagonists (20), serotonin (6), histamine (2), and acetylcholine receptor ligands (1), biogenic amine uptake inhibitors (9), protein kinase modulators (6), and miscellaneous chemistries such as cell cycle regulators and apoptosis inhibitors (7).

Ten “hit” compounds (amitriptyline hydrochloride, (±)-butaclamol hydrochloride, clozapine, doxepin hydrochloride, cis-(Z)-flupenthixol dihydrochloride, methiothepin maleate, mianserin hydrochloride, niclosamide, piceatannol, and resveratrol), in addition to SCH23390 were selected for screen validation assays. These compounds were tested for their activity in cAMP accumulation assays to control for potential “off-target” effects (i.e. chemistries that affect the CRELuc reporter system). Seven of these compounds were potent antagonists of the AaDOP2 receptor, as shown by the dose-dependent reduction of cAMP accumulation relative to the dopamine-stimulated control (Table 3). Three of the compounds (i.e. niclosamide, piceatannol, and resveratrol) showed no significant antagonistic effects against AaDOP2 in the cAMP accumulation experiments, having IC₅₀ values ≧10 μM.

### Confirmation and Secondary In Vitro Assays

The active compounds were also tested against hD₁ to allow for comparisons of relative potency between species (Table 3). These comparisons clearly indicated a unique pharmacology between AaDOP2 and hD₁ with divergent rank order functional potencies that showed no significant correlation (R²<0.15). For example, the prototypical mammalian D₁ antagonist, SCH23390 was greater than 3000-fold more selective for hD₁ than AaDOP2. In contrast, the data also revealed that two structurally-related tricyclic antidepressants (i.e. amitriptyline and doxepin) had more than 30-fold selectivity for AaDOP2 when compared to hD₁. These observations suggest that the significant differences between these receptors could be exploited for the development of AaDOP2-selective compounds.

In Vivo Ae. aegypti Bioassays

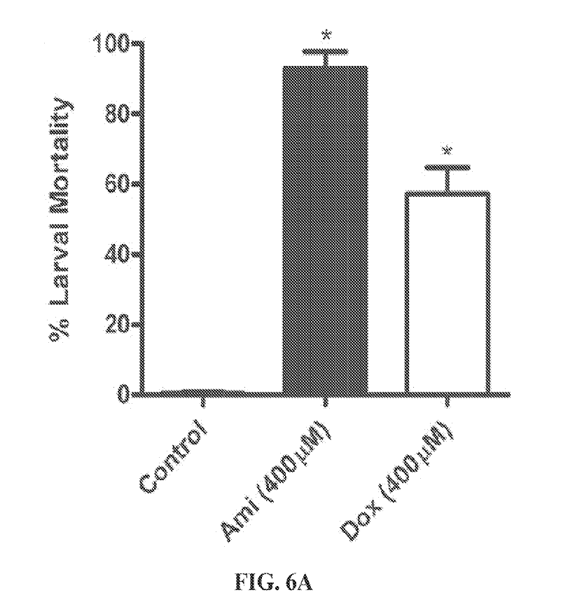

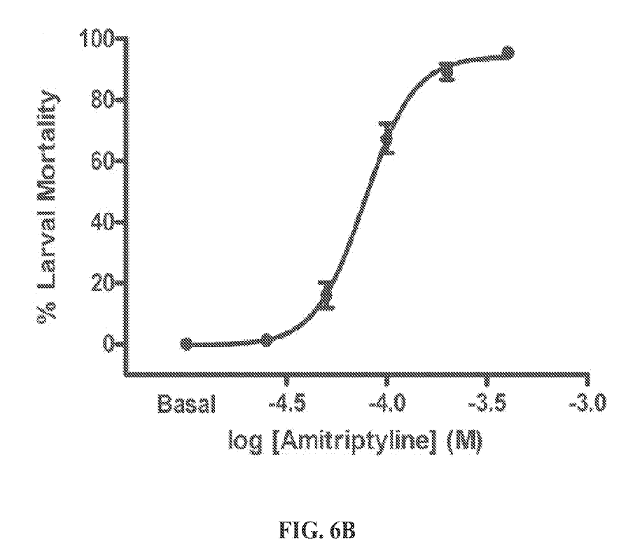

The toxicity of the AaDOP2 antagonist screen hits amitriptyline and doxepin was assessed in Ae. aegypti larval bioassays. These chemistries were selected due to their relatively higher potency at AaDOP2 compared to hD₁ (Table 3). Single dose point assays at 400 μM effective concentration of drug revealed that amitriptyline (93% average mortality) and doxepin (57% average mortality) each caused significant larval mortality (p<0.05) 24 hours post-treatment relative to the water control (0% mortality), whereas no mortality was observed for SCH23390 during this timeframe (data not shown). Dose response experiments were conducted for amitriptyline, which was prioritized due to the rapid and high mortality effect observed in the single-point assays. The toxicity of amitriptyline was dose-dependent, and the LC₅₀ and LC₉₀ values were determined at 78 μM and 185 μM, respectively (FIG. 6).

This work provides the first detailed investigation into the molecular and pharmacological properties of D₁-like dopamine receptors, AaDOP1 and AaDOP2, from the mosquito vector of dengue and yellow fever, Ae. aegypti, and the development of a cell-based screen assay to discover antagonists of AaDOP2. Our study employed a novel pipeline utilizing a “genome-to-lead” approach for the discovery of new chemistries for vector control. This research establishes a basis for improving understanding of mosquito dopaminergic processes in vivo and for chemical screening of these and other receptors characterized in arthropod vectors of human disease, such as in the Lyme disease tick, I. scapularis [36,41]. To our knowledge, Lee and Pietrantonio [46] have published the only other study involving the functional characterization of a biogenic amine-binding GPCR in mosquitoes that was focused on a Gαₛ-coupled serotonin receptor in Ae. aegypti. Furthermore, ligands of only four other cloned GPCRs have been pharmacologically verified in mosquitoes, including those that target an adipokinetic hormone receptor, a corazonin receptor, a crustacean cardioactive peptide receptor [47], and an adipokinetic/corazonin-related peptide receptor in the malaria mosquito, A. gambiae [48].

Typically, insects possess three different dopamine receptors including two D₁-like receptors and a single D₂-like receptor [42]. Here, RT-PCR data were used to validate the two mosquito D₁-like dopamine receptor gene models [10]; this enabled confirmation of intron/exon boundaries and prediction of the complete protein coding regions needed prior to heterologous expression studies. A putative D₂-like dopamine receptor gene (AaDOP3) was also identified in Ae. aegypti [10] although this receptor has not yet been functionally characterized. The RT-PCR studies also demonstrated that transcripts for both D₁-like dopamine receptor genes were detectable in each developmental stage of Ae. aegypti, suggesting the importance of these receptors throughout the mosquito life cycle. Much progress has been made in determining the life-stage and tissue-specific expression dynamics of the orthologous dopamine receptors in D. melanogaster [14,30-31,39,43,49], A. mellifera [40,42,50-53], and most recently in the Lyme disease tick, I. scapularis [41]. Our research will support future complementary studies needed to localize expression of these dopamine receptors in mosquito tissues to gain further insight toward their neurophysiological roles.

The AaDOP1 and AaDOP2 amino acid sequences were compared and analyzed to identify conserved as well as unique features of the receptors. Several characteristics typically associated with biogenic amine-binding GPCRs were evident, including aspartate residues in TM II as well as TM III that are thought to interact with the amine moieties of catecholamines [54]. The conserved serine residues in TM V and aromatic residues in TM V and VI are also potentially important for ligand interaction [55-56]. In both receptors, the conceptual cytoplasmic region of TM III contained the conserved “DRY” motif associated with G-protein coupling [57-58], and a pair of cysteine residues were located in the extracellular loops I and II that may form a disulfide bond for protein stabilization [57,59-60]. Interestingly, the divergent intracellular loop III was predicted to be almost twice as long in AaDOP2 (115 amino acids) than in AaDOP1 (62 amino acids), but the sizes of the carboxyl tail region were similar between these receptors. This corresponded well with the relative sizes of these features in the fruit fly and honeybee orthologs [42]; however, the significance of these characteristics is yet to be determined in the mosquito. Importantly, the AaDOP1 and AaDOP2 sequences were markedly different from the human D₁-like dopamine receptor sequences. Although a modest level of amino acid identity (˜50%) was observed between the TM domains, the N- and C-termini and extracellular and intracellular loop regions were highly divergent (data not shown). These differences suggest that there exists potential for identifying chemistries that are mosquito-specific and, importantly, do not interfere with dopaminergic functioning in humans.

Heterologous expression experiments conducted in HEK293 cells provided experimental evidence that the Ae. aegypti receptors are functional D₁-like dopamine receptors. We measured significant increases in cAMP accumulation following dopamine treatment of cells transiently expressing either AaDOP1 or AaDOP2, suggesting that both receptors are coupled to Gαₛ proteins. This effect was further substantiated in cell lines stably co-expressing either of these receptors and the CRELuc reporter system, as measured by an increase in luciferase activity following dopamine treatment. Future research is needed to determine if these receptors operate through multiple cellular signaling mechanisms, such as was shown for the D. melanogaster dopamine receptor involved with both cAMP and calcium signaling [61].

The stably transformed cell lines were used to compare the pharmacological properties of AaDOP1 and AaDOP2 in response to seven different biogenic amines. For dopamine, we measured EC₅₀ values in the nanomolar range for both AaDOP1 (3.1±1.1 nM) and AaDOP2 (240±16 nM). However, there were differences in the responses of these receptors to the other biogenic amines. AaDOP2 was activated only with dopamine, whereas AaDOP1 was stimulated by dopamine, epinephrine and to a lesser extent, norepinephrine. These results were similar to those reported for the orthologous dopamine receptors in the tick I. scapularis [36,41]. Another difference between AaDOP1 and AaDOP2 was observed regarding constitutive activity. In both transient and stable expression experiments, the AaDOP1 receptor exhibited significant constitutive activity, as determined by the elevated levels of cAMP detected in the absence of a receptor agonist, whereas AaDOP2 did not. Such constitutive activity was also reported for the D₁-like dopamine receptors AmDOP1 of A. mellifera [40], CeDOP1 from the nematode Caenorhabditis elegans [62], Isdop1 of I. scapularis [36], and the human D₅ receptor [63]. Seifert and Wenzel-Seifert [64] proposed that constitutive activity of a GPCR may enable the maintenance of basal neuronal activity, although evidence is needed to support such activity for AaDOP1 in vivo.

The pharmacological properties of AaDOP1 and AaDOP2 were further explored by testing their responses to synthetic dopamine receptor agonists and antagonists. Both receptors were strongly stimulated by the agonist DHX; however, only AaDOP1 significantly responded to the well characterized D₁ agonists SKF81297 and SKF38393. This differential response to the SKF compounds was also observed for the orthologous D₁-like dopamine receptors in the tick I. scapularis [36]. Interestingly, neither of the D. melanogaster D₁-like dopamine receptors was strongly stimulated by SKF38393 [31,39]. Both AaDOP1 and AaDOP2 were inhibited by the antagonist SCH23390, as were the tick D₁-like receptors [36]. This contrasted with the lack of significant inhibition reported by SCH23390 for D-dop1 in the fruit fly [39] and DOP1 of the honeybee [50]. Given the limited number of drugs that have been tested against these receptors, to date, these differential pharmacological responses provide further evidence that it may be possible to discover chemistries that operate specifically at the mosquito dopamine receptors.

Our over-arching goal was to develop a pipeline to identify lead chemistries active at biogenic-amine binding GPCRs in vector arthropods. Broadly speaking, we define a lead chemistry as any molecule, or its analog or derivative, with potential for insecticide development. In our study, this refers to any molecule identified by screening and subsequently confirmed in a variety of “hit-to-lead” assays. The LOPAC₁₂₈₀ library was chosen for our pilot screen because it is enriched with chemistries that influence dopaminergic processes and includes other GPCR-binding ligands. We hypothesized that chemistries that antagonize these dopamine receptors may possess insecticidal properties. Precedent for this notion stems from pest management successes associated with the use of Fipronil and cyclodienes, which antagonize GABA-gated chloride channels and have highly insecticidal properties (Bloomquist, 1996; Casida and Quistad, 1998). This notion was pursued using HEK293 cells stably expressing AaDOP2 because this receptor has a robust response to dopamine and a low constitutive activity, which are properties that aid interpretation of screen data. Our initial screen was directed at the identification of AaDOP2 antagonists; the success of this experiment justifies expanded screening to explore the antagonist chemical “space”, and with assay modification, screens to detect agonists active at this receptor. Moreover, development of the AaDOP1 assay would enable comparative screens against LOPAC₁₂₈₀ chemistries.

Of the 51 hit AaDOP2 antagonists identified in the LOPAC₁₂₈₀ library, 20 (39%) are known antagonists of mammalian dopamine receptors. A majority of these chemistries fall into the benzodiazepine, phenothiazine or thioxanthene classes that in other systems are known to bind other biogenic amine receptors. Included were ligands selective for D₁- and D₂-like dopamine receptors in mammalian systems, as well as several non-dopamine receptor selective compounds such as (±)-butaclamol, cis-(Z)-flupenthixol, and the atypical antipsychotic, clozapine. These three compounds were tested in a dose-response format for their ability to inhibit dopamine-stimulated cAMP accumulation. The IC₅₀ values demonstrated the following rank order of potency clozapine>cis-flupenthixol>butaclamol. The next largest grouping of identified compounds includes inhibitors of the biogenic amine transporters (9 compounds, 18%). Several serotonin receptor antagonists (6 compounds, 12%) were identified as well. Follow-up dose response studies with selected chemistries from the identified transport inhibitors and serotonin antagonists (i.e. methiothepin, mianserin, amitriptyiline, and doxepin) revealed that these compounds were potent antagonists at the AaDOP2 receptor and were much more potent than the prototypical D₁ antagonist, SCH23390 (Table 3). The antagonistic activity of these ligands is not completely surprising; the National Institute of Mental Health's Psychoactive Drug Screening Program (NIMH-PDSP) database reports Kᵢ values for the human D₁-like dopamine receptors at 80-900 nM (http://pdsp.med.unc.edu/). However, these observations, combined with the dopamine antagonist screen results, indicate that well studied and clinically used compounds could be used to target invertebrate GPCRs. In fact, a number of the chemistries identified in our screen have been used in humans for decades, suggesting the possibility of “drug repurposing” as insecticides. Further precedent for the concept of insect-specific chemistries can be drawn from the fact that a number of insecticides (e.g., pyrethroids and fipronil) are considerably more selective at invertebrate as opposed to mammalian targets [65]. The screen also identified multiple protein kinase modulators and several agents that regulate germane cellular functions that presumably inhibit the CRE response via non-AaDOP2 mechanisms. Support for this hypothesis was demonstrated in the direct measurement of cAMP accumulation experiments, where resveratrol, pieacetannol, and niclosamide each lacked activity. The remaining three “hit” compound classes included antagonists of either histamine or muscarinic acetylcholine receptors, and this likely reflects the lack of receptor selectivity for these ligands.

The LOPAC₁₂₈₀ library includes several known antagonists of mammalian dopamine receptors that did not qualify as hits in our screen. In part, this can be explained by the fact that we used a highly stringent cut-off to signify antagonistic activity at AaDOP2. Had we reduced the stringency to select for hits with an antagonistic effect equivalent to that of SCH23390+6 standard deviations (69% inhibition), our screen would have returned an additional 13 hit chemistries, including compounds predicted to have a modest antagonistic effect at AaDOP2 and those that are more selective for D₂-like dopamine receptors. Considering the substantial divergence between the mosquito and human D₁-like dopamine receptor sequences, there is a strong possibility that a subset of the “non-hit” dopamine receptor antagonists are not active at the mosquito receptor. In support of this, the prototypical mammalian D₁ antagonist, SCH23390, was greater than 3000-fold more selective for hD₁ than AaDOP2. Although our comparison data set is limited to only eight compounds, these experiments suggest a very divergent pharmacology between these human and mosquito dopamine receptors. Thus, our study provides a foundation for subsequent comparative pharmacological analyses of the mosquito and human dopamine receptors.

We observed a strong correlation between in vitro and in vivo data. The AaDOP2 antagonist screen hits, amitriptyline and doxepin, caused significant lethality in the mosquito larval assay. Our finding that these drugs each have a relatively higher potency at the mosquito dopamine receptor than at hD₁ has implications for the identification of arthropod-selective chemistries. Drugs with minimal or no impact on the neurological functioning of humans or other vertebrate species are particularly desirable as prospects for insecticide development. Conversely, SCH23390, which is active at AaDOP2 only in the micromolar range and was several fold more selective for hD₁ in cAMP assays, did not cause significant larval mortality at 24 hr.

The success of this initial chemical library screen in identifying new mosquitocidal chemical leads justifies the pursuit of an expanded high-throughput screening effort involving thousands or hundreds of thousands of chemistries against mosquito dopamine receptors. Our platform is also amenable for the screening of agonist chemistries active at these mosquito dopamine receptors, as well as for Gαₛ-coupled biogenic amine targets of other vector arthropods, and also could be modified to screen Gα_{i/o}-coupled receptors [66]. Importantly, the identification of lead AaDOP2 receptor antagonistic chemistries provides a basis for investigating the effect of these or related compounds on mosquito dopaminergic processes in vivo [67]. Follow-up research is needed to determine the precise mechanism(s) of amitriptyline- and doxepin-induced mortality in A. aegypti larvae. Further work is also needed to determine if these chemistries and associated derivatives or analogs identified by chemical screens possess the properties desired of an insecticide (e.g. bioavailability, in vivo potency/toxicity, suitable half-life, lack of effects on non-target organisms, suitability for synthesis and formulation). Molecular modeling of three dimensional GPCR structures and their binding capabilities, as reported for an adipokinetic hormone receptor in A. gambiae [68] and a tyramine receptor in the moth Plodia interpunctella [69], may facilitate in silico chemical screening [70] and ligand-receptor studies that permit the design or refinement of lead molecules active at mosquito GPCRs.

Historically, multiple neuroactive processes in arthropods have been exploited for pest control using insecticides such as chlorinated hydrocarbons, organophosphates, methylcarbamates, pyrethroids, amidines, and phenylpyrazoles. Resistance involving each of these classes (the vast majority of which operate by affecting ion channels and neurotransmitters) has been documented. The development of new mode-of-action insecticides could improve our arsenal against mosquito populations that have developed resistance to existing chemical formulations [1]. We suggest that the two dopamine receptors characterized here, as well as other biogenic amine-binding GPCRs [72-73], represent promising targets for new insecticide research, due to their presumably central roles in insect neurobiology. This “proof-of-concept” study sets the stage for target-specific approaches for vector control. Such efforts, in parallel with activities of organizations such as the Innovative Vector Control Consortium, may help to realize the goal of delivering new insecticides for reduction of vector-borne diseases [2].

Development of a structure-activity relationship (SAR) for AaDOP2 antagonist molecules has been pursued on two main fronts: (1) cell-based SAR studies to identify novel chemistries that antagonize the AaDOP2 target in vitro, and (2) complementary whole-organism in vivo SAR studies to identify those chemistries that cause mortality of key insect pests and have greatest potential for insecticide development.

### In Vitro AaDOP2 Antagonist SAR Studies

cAMP Accumulation Confirmation Assays.

Confirmation of screen hits has been carried out using a secondary cellular assay and evaluation of in vitro potency of antagonist molecules (i.e., the inhibitory concentration or IC50 value). An in vitro SAR analysis of hits from the LOPAC1280 screen for antagonists of the AaDOP2 receptor using a Homogenous Time Resolved Fluorescence (HTRF) cAMP accumulation assay (Cisbio Dynamic 2) was completed. These assays were performed as described in Meyer [84] with modifications to miniaturize the assay to 384-well format. We evaluated the potency of more than 40 potential antagonist chemistries at the AaDOP2 target (Table 9). Chemistries were tested in dose-response experiments in the presence of 304 dopamine in AaDOP2 expressing cells in comparison to the prototypical mammalian dopamine antagonist SCH23390 and amitriptyline. The data in Table 9 are representative of three independent experiments performed in duplicate.

### High Throughput Screening of the AaDOP2 Target to Identify Novel Antagonist Chemistries.

To explore more unique chemical space and identify highly novel chemistries that could be pursued as leads for insecticide development, an additional 6,067 compounds in our Cisbio high-throughput in vitro screen were screened. Three libraries were screened in total; these were the NIH Clinical Collection and Clinical Collection 2 (NCC and NCC 2; 727 small molecules with a history of use in human clinical trials), the Spectrum Collection (MicroSource Discovery Systems Inc., Gaylordsville, Conn.; 2,320 compounds; 60% drugs, 25% natural products and 15% other bioactive molecules, including insecticides), and the TimTec Natural Derivatives Library NDL-3000 (TimTec LLC, Newark, Del.; 3,040 compounds; synthetic compounds and synthetically modified pure compounds). Screens were performed as per methods described in Meyer [81] and Ejendal [78] with modifications and appropriate validation procedures to execute the screening assays in 384 well format using the Cisbio HTRF cAMP assay.

### In Vivo AaDOP2 Antagonist SAR Studies

In Vivo Aedes aegypti High-Throughput Screen.

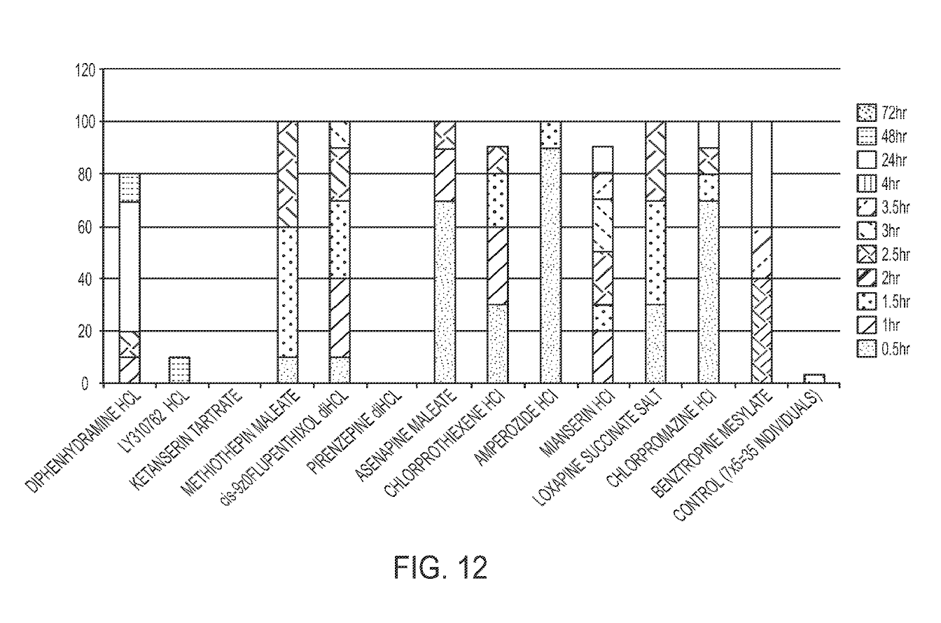

As a second step, the in vivo activity of chemistries identified via in vitro studies was evaluated. Initially, chemistries were evaluated in a high-throughput in vivo assay against L3 stage larvae of the yellow fever mosquito, Ae. aegypti. The chemistries were tested in duplicate at a single point dose of 40004 in 24-well plate (BD Bioscience, San Jose, Calif.) assay. Briefly, chemistries were re-suspended in water and added to wells containing five Ae. aegypti L3 larvae to achieve a final concentration of 400 μM per well in 1 ml sterile RO water. The plates were incubated at 22° C. and the assay was scored at 30 minutes and 1, 1.5, 2, 2.5, 3, 24, 48 and 72 hours. The screen was performed as a double-blind experiment and enables a rapid evaluation of the in vivo activity of multiple water-soluble chemistries, and the prioritization of molecules that exhibit rapid and/or high mosquito mortality. The toxicity of 25 AaDOP2 antagonist chemistries using this assay is presented in for three independent screens (n=3) in Tables 8 and 10 and FIG. 12.

Ae. aegypti Concentration Response Curves and Time Course Assays.

Chemistries that cause rapid and high mortality of mosquitoes in our high-throughput in vivo screen were subsequently evaluated in concentration response assays to determine LC50 (lethal concentration) and LT50 (lethal time) values as described by Meyer et al [81], with the results presented in Tables 11 and 15. Briefly, chemistries were diluted to appropriate concentration in water and added to the wells of 24-well plates containing 5 L3 stage Ae. aegypti larvae to achieve a final concentration of 400, 200, 100, 50 and 25 μM in 1 ml sterile RO water. Chemistries were tested in quadruplicate at each concentration. The plates are incubated at 22° C. and percent mortality was assessed at 30 minutes and 1, 1.5, 2, 2.5, 3, 24, 48 and 72 hours. Results reported here represent a minimum of three independent CRC experiments (i.e., n=3).

### Additional Insect In Vivo Toxicity Assays.

A series of whole organism bioassays was developed to further evaluate the toxicity of chemistries to a range of agricultural, veterinary and public health pests and non-target insects (Tables 12 and 13). These assays are designed to rapidly explore the biological activity spectrum of chemistries as well as to facilitate initial investigations of the mode of action of insecticidal compounds. Briefly, assays were developed for the German cockroach (Blatella germanica, urban pest), termite (Reticulitermes flavipes; urban pest), lone star tick (Amblyomma americana; veterinary and public health pest), soybean aphid (Aphis glycines, agricultural pest) and honeybee (Apis mellifera; beneficial, non-target insect). The assays were designed to test the toxicity and speed of kill of multiple chemistries on contact. Each of these assays was performed as a single point dose experiment in triplicate using either topical application of compound to the ventral thorax (1 μl of a 200 μM compound solution in 1:1 v:v DMSO:EtOH vehicle; 50-80 μg effective dose depending on compound) as is the case for our cockroach and honeybee assays, or 10 second immersion in a solution of the compound diluted in water or DMSO:EtOH to a final concentration of 100 μM. The assays were conducted in 24-well plate format with mortality assessment following incubation at 22° C. as described above. Commercial insecticides were incorporated in these assays to provide a positive control and permit initial comparative analyses. Lead chemistries identified in our in vivo screen have been evaluated in the cockroach and termite assays.

### In Vitro AaDOP2 Antagonist SAR Studies

cAMP Accumulation Confirmation Assays.

An in vitro SAR for antagonists of the AaDOP2 receptor using a Homogenous Time Resolved Fluorescence (HTRF) cAMP accumulation CRC assay (Cisbio Dynamic 2) has been completed. The potency (IC50 value) of more than 40 potential antagonist chemistries at the AaDOP2 target (Table 9) was evaluated, identifying fourteen chemistries with high potency (IC50<100 nM), 12 chemistries with moderate potency (IC50 between 100 nM-1 μM), and eight chemistries with low potency (IC50>1 μM). Six antidepressant molecules (e.g., fluoxetine) and the insecticide molecule, Amitraz, showed no inhibition in the assay. From this preliminary SAR, we have identified a total of five general chemical scaffolds (i.e., dibenzocycloheptane derivatives, phenothiazine derivatives, thioxanthene derivatives, butyrophenone derivatives, and diphenyl amine-containing compounds) that provide the basis for an expanded SAR. It is also noteworthy that within several of the chemical scaffolds, are multiple distinct and more specific chemical classes. For example, the dibenzocycloheptane derivatives also contain dibenzazepines and dibenzodiazepines, and the diphenylamines contain three unique diphenyl structures (i.e., diphenylpiperazine, diphenylpiperidine, and diphenylmethoxy derivatives).

The initial SAR analysis in Table 9 provides the following information on ligand requirements: The amine state contributes to the determination of antagonist potency at the AaDOP2 receptor. For example, compounds with tertiary amines (clomipramine, imipramine, amitriptyline, and loxapine) are approximately five to 100 fold more potent than the secondary amine analogs of these compounds (norclomipramine, desipramine, nortiptyline, and amoxapine, respectively). Furthermore, clomipramine is approximately six fold more potent than imipramine, suggesting that aromatic ring substituents can enhance the activity of the identified antagonists. It should also be noted that the data suggest that the central ring is not necessary for antagonist activity, as risperidone, benztropine and amperozide are moderately potent. However, compounds with six or seven-membered central rings are generally the most potent AaDOP2 receptor antagonists, suggesting a relationship between conformational rigidity and antagonist activity. Interestingly, a conformationally rigid molecule, asenapine, is one of the two most potent compounds tested to date. The structural features noted above can be used to assist in guiding future studies.

### High Throughput Screening of the AaDOP2 Target to Identify Novel Antagonist Chemistries.

In total, 93 new hit compounds were identified in our small molecule screens of the AaDOP2 target against the NIH Clinical Collection, Spectrum Collection and TimTec NDL-3000 library (Tables 9 and 14). Specifically, forty-five hit compounds (including 24 new hits) in the NIH Clinical Collections that caused ≧80% inhibition of the AaDOP2 receptor. Seventeen of these hits were previously identified in a screen of the LOPAC1280 library and four additional compounds had been evaluated in follow-up CRC assays due to their structural similarity to LOPAC1280 library hits. 4 antagonist compounds (58 new hits) were identified from the Spectrum Collection that caused ≧80% inhibition of the AaDOP2 target. Thirty-six of these compounds were previously identified in the LOPAC1280 screen. 11 compounds were identified from the TimTec NDL-3000 library that caused ≧60% inhibition of the AaDOP2 target. Within these 11 compounds at least three new scaffolds have been identified, including quinazoline, benzodiazoxide and indole derivatives.

Quantities of a selection of hit chemistries for follow-up in vitro CRC confirmation assays were obtained to confirm screen hits and evaluate the comparative potency (IC50 values) of compounds. Additionally, a screen of 10,000 chemistries from the ChemDiv library (San Diego, Calif.) was completed. An additional 62 hits were identified from this screen and the top 28 of these were obtained for follow-up confirmation assays. The results of the initial 10,000 compound screen were subjected to a chemoinformatics analysis. The results of analysis revealed a number of promising trends enabling the development of a preliminary molecular model of the AaDOP2 receptor. The results also identified three novel putative chemical scaffolds for AaDOP2 (i.e. piperazinylpyrazolopyrimidines, aryldiaminopyrimidines, and hexahydrothienopyridines).

Thus a total of 17,367 compounds have been screened at the AaDOP2 target, and more than 10 chemical scaffolds and 150 unique hits via the “hit-to-lead” process [81] have been pursued.

### In Vivo AaDOP2 Antagonist SAR Studies

In Vivo Aedes aegypti SAR Screen.

Twenty-five chemistries were evaluated for toxicity to Ae. aegypti larvae in a high-throughput in vivo assay (Tables 8 and 10; FIG. 12). Ten chemistries (asenapine, chlorpromazine, benztropine, methiothepin, cis-flupenthixol, chlorprothixene, loxapine, mianserin, amperozide and clomipramine) were identified that caused 70-100% mosquito mortality within the first 24 hours post-exposure compared to the water-only control (FIG. 12).

These 10 chemistries caused higher mortality at 24 hours and were faster acting than amitriptyline. Three chemistries (asenapine, chlorpromazine, and amperozide) were extremely fast acting and caused greater than 70% mortality of the mosquito population within 30 minutes and five chemistries (cis-flupenthixol, chlorprothixene, mianserin, loxapine and methiothepin) caused greater than 70% mortality within three hours. These 10 chemistries were selected for in vivo concentration response analyses and additional insecticide bioassays (see Sections 2.2 and 2.3). Additionally, five chemistries with moderate mosquito toxicity (i.e., 40-70% mortality at 24 hours post exposure) and nine chemistries with limited or no toxicity to mosquito larvae (i.e., 0-40% mortality at 24 hours) were identified. The in vivo SAR data showed a strong correlation with the in vitro SAR results (see Tables 9 and 10), suggesting a possible mechanism for the chemistries to act by disrupting the AaDOP2 target in vivo. However, amperozide, which caused rapid and high mosquito mortality, is a weak inhibitor of AaDOP2 in vitro (570±110 nM), suggesting that other mechanisms may also be involved.

Ae. aegypti Concentration Response Curve and Time Course Assays

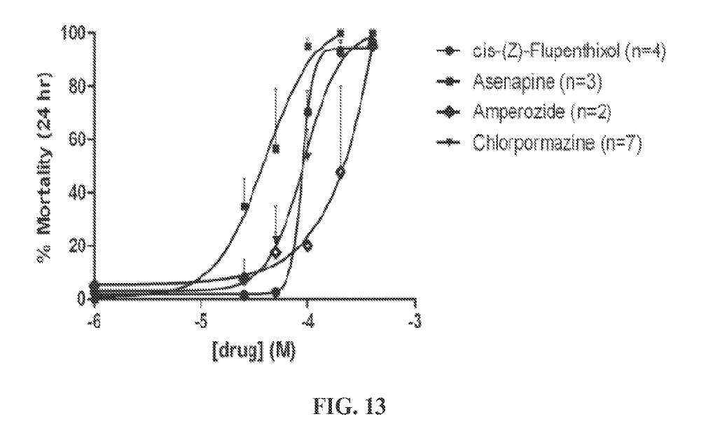

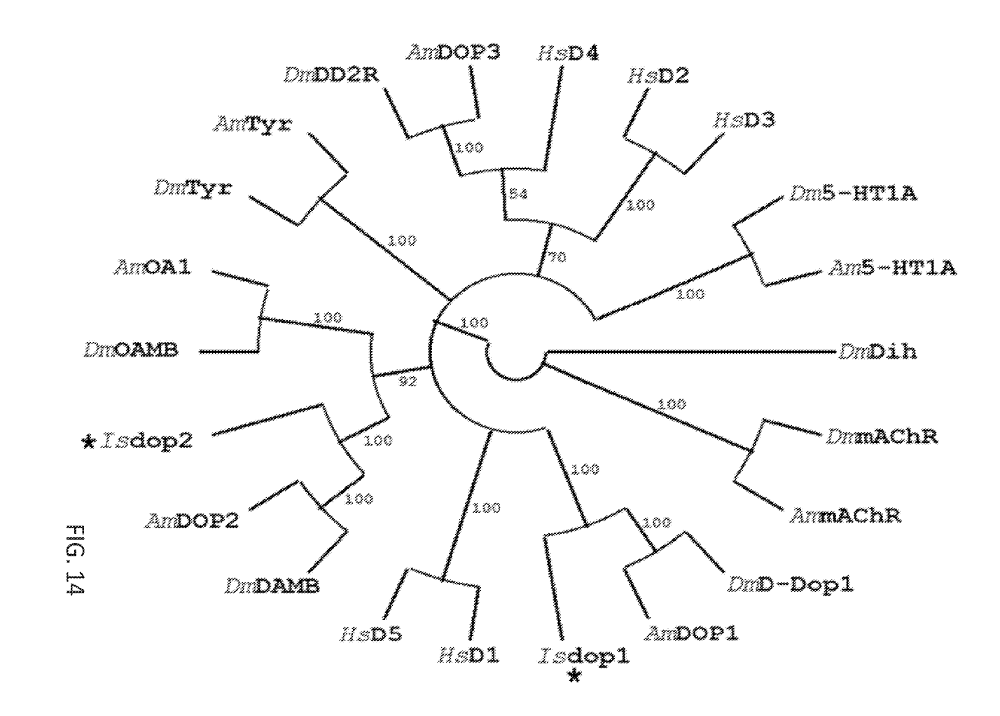

The lethal concentration (LC50; Table 11, FIG. 13) and lethal time (LT50; Table 15) values for our ten lead chemistries were also evaluated. The LC50 values of chemistries we have evaluated on this project to date range from 40 μM (asenapine) to 92 μM (chlorpromazine). Of note, the LC50 value of asenapine is approximately half that of amitriptyline. This represents a significant increase in toxicity and highlights the importance of the approach for identifying molecules with potential for insecticide development. Toxicity in the micro-molar range is typical for unformulated chemistries. Following chemical formulation, improvements in potency are expected.

### Additional Insect In Vivo Toxicity Assays.

Several chemistries (amitriptyline, chlorpromazine and cis-(z)-flupenthixol) that cause moderate toxicity (25-35% mortality) to cockroaches on contact within three to five days post dose (Table 12) have been identified. Using a published feeding assay [80] that involves incorporation of test compounds into an inert bait matrix, no significant oral toxicity of lead chemistries to cockroaches (Table 16) was demonstrated, suggesting that mode of action is associated with absorption via the cuticle. Importantly, four lead chemistries (cis-(z)-flupenthixol, chlorpromazine, amitriptyline and amperozide) were shown to cause rapid toxicity to adult termites within 24 hours, with significant mortality (70-100%) of the test population within five days (Table 13). Although not wishing to be bound by any theory, one possibility is that the smaller biomass and thinner, less melanized cuticle of R. flavipes in comparison to B. germanica significantly increases the bioavailability of test compound and contributes to the higher mortality we observe in termite assays. The insect bioassay results expand the known activity spectrum of our chemistries to three insect orders, namely Diptera (flies and mosquitoes), Blattodea (cockroaches) and Isoptera (termites), thus suggesting significant commercial potential of resultant insecticides

Materials and Methods—Ixodex scapularis.

### Sequence Analyses

The intronless genes IscaGPRdop1 and IscaGPRdop2 (referred to henceforth as Isdop1 and Isdop2) correspond to the automated gene models ISCW001496 and ISCW008775 from the I. scapularis genome assembly, respectively, which were downloaded from VectorBase (http://www.vectorbase.org/index.php) (Lawson et al., 2009). These genes were identified with blastn searches (Altschul et al., 1997) using the D. melanogaster dopamine receptor sequences D-Dop1 (Gotzes et al., 1994) and DopR99B (DAMB) (Feng et al., 1996; Han et al., 1996) as queries against the I. scapularis genome assembly. Isdop1 encodes a protein of 425 amino acids and is located on scaffold DS648196 of the genome assembly at positions 133,404e134,681. Isdop2 is 457 amino acids in length and located on scaffold DS812273, spanning from base pair positions 248,624e247,251. Gene annotations were managed with Artemis software (http://www.sanger.ac.uk/resources/software/artemis/) (Rutherford et al., 2000).

A neighbor joining phylogenetic analysis was conducted to determine the relationships of the full-length deduced amino acid sequences for Isdop1 and Isdop2 with multiple biogenic amine receptors from the insects D. melanogaster and A. mellifera, and the human dopamine receptors. Sequence alignments were performed using ClustalW 1.83 (Chema et al., 2003), and tree construction was conducted with PAUP 4.0b4a (Swofford, 2001). Support for branches in the tree was generated with the bootstrap method (100 replicates) in PAUP.

Multalin software (Corpet, 1988) was used to align conceptual amino acid sequences of the dopamine receptors from I. scapularis, D. melanogaster, A. mellifera and Homo sapiens for comparative analyses of key structural components. To provide support for identification of putative TM domains, hydrophobicity plots were generated with ProtScale software (Kyte and Doolittle, 1982) available at the ExPASy Proteomics Server, Swiss Institute of Bioinformatics (http://ca.expasy.org/tools/protscale.html). Kinase-specific protein phosphorylation sites for protein kinase A and protein kinase C were predicted using the NetPhosK 1.0 Server, Technical University of Denmark (http://www.cbs.dtu.dk/services/NetPhosK/). Putative 1-4 N-linked glycosylation sites were identified with the NetNGlyc 1.0 Server, Technical University of Denmark (http://www.cbs.dtu.dk/services/NetNGlyc/) and EnsembleGly (http://turing.cslastate.edu/EnsembleGly/) (Caragea et al., 2007). Putative palmitoylation sites were identified with CSS-Palm 2.0 (http://csspalm.biocuckoo.org/online.php) using a medium threshold (Ren et al., 2008).

### Gene Expression

Blastn searches (Altschul et al., 1997) were conducted at Gen-Bank and VectorBase to search for expressed sequence tags (ESTs) corresponding to the Isdop1 and Isdop2 sequences. For gene expression, a pooled sample of total RNA was isolated from 10 adult female I. scapularis with TRIzol Reagent (Invitrogen, Carlsbad, Calif.). Total RNA was then treated with RNase-Free DNAse (QIAGEN, Valencia, Calif.) prior to cDNA synthesis. Because both Isdop1 and Isdop2 are single exon genes, it was not possible to design primers that would span intronic regions as a control for contaminating genomic DNA. To circumvent this issue, 30-rapid amplification of complementary DNA ends (30-RACE) experiments were designed according to the directions provided in the GeneRacer_Kit (Invitrogen, Carlsbad, Calif.). Target amplification of synthesized cDNA was conducted using two gene-specific forward primers (nested PCR) and reverse primers (provided in kit) which annealed to the polyadenylated 30 tail. Gene-specific primers used for amplification of the 30 end of Isdop1 included Ixodes_Dop1_{—}30RACE_{—}1F (50-CCTATCGCACCAAGAGAAGCACCATTTG-30) and Ixodes_Dop1_{—}30 RACE_{—}2F (50-GGATGCCGAAGCAACAACAACACTG-30), respectively. Gene-specific primers used for amplification of the 30 end of Isdop2 included Ixodes_Dop2_{—}30RACE_{—}1F (50-TGGATCAACTCCGGCATGAAC CCCATCA-30) and Ixodes_Dop2_{—}30RACE_{—}2F (50-ACCGCCTCCGACGCATCATCAAGGAAGA-30). To amplify cDNA produced in the 30-RACE procedure, a standard PCR protocol was used according to the reagent manufacturers' instructions (Bioline USA Inc., Randolph, Mass.). Parameters used for cDNA amplification in both the initial and nested PCR steps included a denaturation step of 94_C for 2 min followed by a modified touchdown PCR including (i) 5 cycles of denaturation at 94_C for 30 s, primer annealing at 70_C and extension at 70_C for 30 s, (ii) 5 cycles of denaturation at 94_C for 30 s, primer annealing at 65_C and extension at 68_C for 30 s, and (iii) 25 cycles of denaturation at 94_C for 30 s, primer annealing at 60_C and extension at 68_C for 30 s. A final extension period of 10 min was conducted at 68_C. Amplification products were separated on 1% TBE gels and compared by size to DNA Hyper-Ladder I (Bioline USA Inc., Randolph, Mass.), and amplicons of interest were excised and extracted from the gel using the Qiagen Gel Extraction Kit (Qiagen Valencia, Calif.). Purified DNA was cloned with the pCR 2.1-TOPO TA cloning kit using One Shot_Top 10 F′ chemically-competent Escherichia coli cells (Invitrogen, Carlsbad, Calif.). Following transformation, cells were grown in 10 cm petri dishes containing Luria Bertani (LB)-agar media+50 μg/ml kanamycin+10 ml X-Gal/IPTG solution (Bioline USA Inc., Randolph, Mass.) to permit blue/white colony selection. Randomly-selected clones were grown in 5 ml LB media+50 μg/ml kanamycin for 16-18 h at 37_C. Plasmid DNA was isolated using the QIAprep spin miniprep kit (Qiagen, Valencia, Calif.), and DNA sequencing of four independent clones for each cloned amplicon was conducted at the Purdue University Genomics Core Facility. A consensus sequence for each cloned amplicon was produced by first aligning the sequences with Multalin software (Corpet, 1988) and then using a majority-rule criterion at each base pair position to filter out potential sequencing errors or polymorphisms. Consensus sequences were compared to their corresponding regions in the I. scapularis genome sequence assembly for calculations of percent nucleotide identities.

### Pharmacological Characterization

Clones corresponding to the coding regions of Isdop1 and Isdop2 were produced by synthesis (GenScript, Piscataway, N.J.) according to their gene models (see above). The partial Kozak transcriptional recognition sequence “CACC” was synthesized directly upstream of the transcription initiation codon for each gene. Each gene was first cloned into the vector pUC57 and then subcloned into the expression vector pcDNA3.1+ (Invitrogen, Carlsbad, Calif.) by Gen-Script (Piscataway, N.J.).

To functionally characterize Isdop1 and Isdop2, heterologous expression experiments were conducted in HEK 293 cells (ATCC, Manassas, Va.) using transient transfections as follows. HEK 293 cells were cultured in Dulbecco's modified Eagle Medium (DMEM) (Sigma, St. Louis, Mo.) supplemented with 5% bovine calf serum (BCS), 5% fetal clone I serum (Hyclone, Logan, Utah), and 1% AntieAnti (Invitrogen, Carlsbad, Calif.) in a humidified incubator at 37_C with 5% CO₂. HEK 293 cells were seeded in 48 well cluster plates (BD Falcon, Franklin Way, N.J.) and transiently transfected with 200 ng of the Isdop1 or Isdop2 construct DNA, or with the vector pcDNA3.1+ alone in the controls, and 0.5 ml Lipofectamine-2000 (Invitrogen, Carlsbad, Calif.) per well. Forty-eight hours post-transfection, the media was decanted and replaced with assay buffer (Earle's balanced salt solution supplemented with 2% BCS, 0.02% ascorbic acid, 15 mM HEPES and 0.5 mM 3-isobutyl-1-methylxanthine) containing each drug (see below) at the concentrations indicated in Table 3 and FIG. 3. To allow for cAMP accumulation, cells were placed at 37_C for 15 min, and the reaction was stopped by decanting the assay buffer and adding 100 ml ice-cold 3% trichloroacetic acid. Accumulation of cAMP was analyzed using a competitive binding assay as described by Przybyla et al. (2009). Briefly, aliquots (12 ml) of the cell lysates were added in duplicate to assay tubes. Subsequently, a final concentration of 2 nM [3H]cAMP (Perkin Elmer, Boston, Mass.) in cAMP binding buffer (100 mM TriseHCl pH 7.4, 100 mM NaCl, 5 mM EDTA) and cAMP binding protein, also suspended in cAMP binding buffer, were added to each tube and the samples were incubated on ice for 2 h. Using a 96-well Packard Filtermate harvester (PerkinElmer, Shelton, Conn.), the samples were transferred to filter plates (Millipore, Billerica, Mass.). The filters were dried overnight, 50 ml of MicroScint 0 scintillation fluid (PerkinElmer, Shelton, Conn.) was added per well, and the plates were subjected to liquid scintillation counting on a Packard TopCount scintillation detector (PerkinElmer, Shelton, Conn.). The concentrations of cAMP in each sample were calculated from a standard curve of cAMP, added at 0.1e100 pmol cAMP per assay tube.

The drugs used for pharmacological studies included dopamine hydrochloride, histamine dihydrochloride, 5-hydroxytryptamine hydrochloride, (_) octopamine hydrochloride, and tyramine hydrochloride (SigmaeAldrich, St. Louis, Mo.) and (_)-epinephrine bitartrate and L (_)-norepinephrine bitartrate (Research Biochemical International, Natick, Mass.). To block epinephrine and norepinephrine activation of endogenous b adrenergic receptors in HEK cells, propranolol (Research Biochemical International, Natick, Mass.) was included in experiments at 300 nM (results summarized in Table 19). The synthetic dopamine receptor agonists that were tested included SKF38393 and SKF81297, and the antagonists were SCH23390 and (+)-butaclamol (SigmaeAldrich, St. Louis, Mo.). At least three independent experiments were conducted for each pharmacological assay.

To expand on our initial findings measuring cAMP responses in transiently-expressing cells and facilitate pharmacological studies more amenable to a high-throughput format, we developed stable cell lines co-expressing either Isdop1 or Isdop2 and a cyclic AMP Response Element/Luciferase (CRE-Luc) reporter system, as previously described by Przybyla et al. (2009). To test this system for each I. scapularis receptor, we conducted two different reporter assays consisting of dose-response curves for dopamine, epinephrine, and norepinephrine and an assay involving the antagonist SCH23390 (10 μM) in combination with dopamine (1 μM). Cells previously transfected with the CRE-Luc reporter construct (HEK CRE-Luc cells) were transfected in a 10 cm dish with 15 μl Lipofectamine-2000 and 3 μg of either the Isdop1 or Isdop2 constructs described above. Clones were selected in Geneticin (SigmaeAldrich, St. Louis, Mo.) and screened for receptor function in response to dopamine. The resulting clones were maintained as described for the wild-type HEK 293 cells above, with the addition of puromycin (2 μg/ml) and Geneticin (300 μg/ml). For the receptor reporter assays, approximately 20,000 cells were seeded per well in white clear-bottomed 384-well plates (Nunc, Fisher Scientific, Pittsburgh, Pa.) and incubated overnight. The cell media was decanted and replaced with 50 μl Opti-MEM (Invitrogen, Carlsbad, Calif.) supplemented with 0.02% ascorbic acid containing increasing concentrations of the indicated biogenic amine. After 2 h incubation in a humidified incubator at 37_C with 5% CO₂, the assay buffer was decanted, 20 μl PBS and 15 μl Steadylite HTS (PerkinElmer, Waltham, Mass.) were added per well, and the plate was incubated on a shaker for 5 min at room temperature. Luminescence was detected on a VictorLight (PerkinElmer, Shelton, Conn.) as counts per second (CPS). Each condition was carried out in at least triplicate. The GraphPad Prism software (GraphPad Software Inc., San Diego, Calif.) was used for statistical analysis of pharmacology data. Statistical analyses included either a Student's t-test or a one-way ANOVA followed by Dunnett's post-hoc test as indicated in the figure legends. p values <0.05 were considered statistically significant.

Results for Ixodes scapularis

### Sequence Analyses

A neighbor joining analysis was conducted to investigate the relationships of the deduced amino acid sequences of each putative I. scapularis dopamine receptor with multiple biogenic amine receptors from D. melanogaster, A. mellifera and the human D1- and D2-like dopamine receptors (FIG. 14). The Isdop1 sequence was joined in a Glade (bootstrap ¼ 100) with the D1-like dopamine receptor sequences D-Dop1 of D. melanogaster (Gotzes et al., 1994) and DOP1 of A. mellifera (Mustard et al., 2003). Isdop2 was included in a separate Glade (bootstrap ¼ 100) with the insect D1-like dopamine receptors (INDRs) (Mustard et al., 2005) that included DopR99B (DAMB) of D. melanogaster (Feng et al., 1996; Han et al., 1996) and DOP2 of A. mellifera (Mustard et al., 2003), and was related to the octopamine receptors OAMB (Han et al., 1998) and AmOA1 (Grohmann et al., 2003) from D. melanogaster and A. mellifera, respectively (bootstrap ¼ 92). ISDOP1 is presented as SEQ. ID NO. 5, and Isdop2 as SEQ. ID NO. 7.

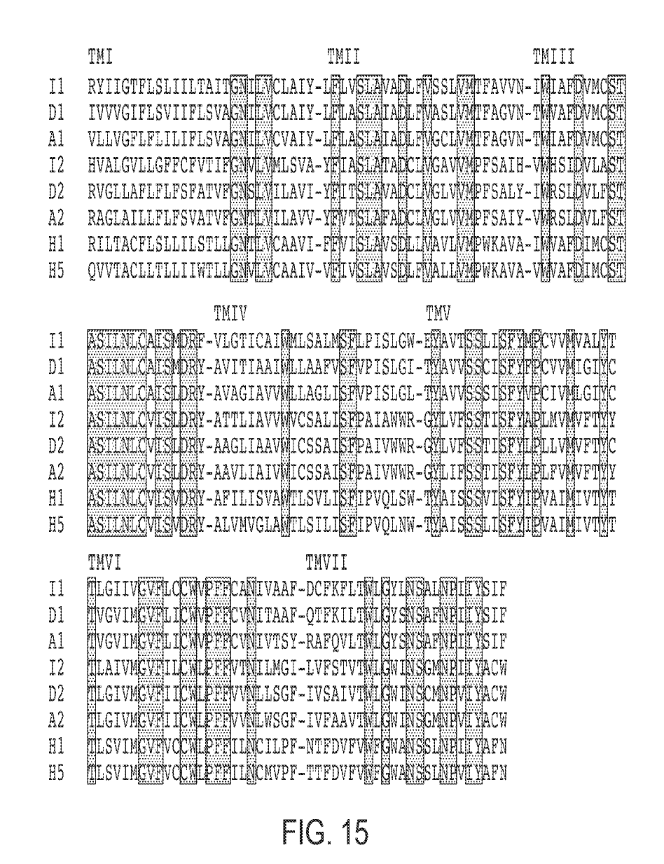

Initially, the seven TM domains of Isdop1 and Isdop2 were identified through comparisons to the TM domain sequences from their putative orthologs in D. melanogaster (Gotzes et al., 1994; Feng et al., 1996) (FIG. 15). For both Isdop1 and Isdop2, these TM domain predictions were also supported by hydropathy analysis (Kyte and Doolittle, 1982; Probst et al., 1992). An alignment of putative TM domains was produced to analyze receptor structure and support predictions of gene orthology. Thirty-three percent of the amino acids composing the TM regions of all identified D1-like receptors in I. scapularis, D. melanogaster, A. mellifera, and H. sapiens were identical, highlighting the similarity among these receptors from distantly related species (shaded residues: FIG. 15). The amino acids composing the predicted TM regions of Isdop1 and Isdop2 were 47% identical. Isdop1 is likely orthologous to D-Dop1 (D. melanogaster) and DOP1 (A. mellifera) in having 72% and 65% amino acid identities in the aligned TM regions, respectively (Table 17). Similarly, the predicted orthologs of Isdop2 are DopR99B (DAMB) (D. melanogaster) and DOP2 (A. mellifera), based on their respective 74% and 74% amino acid identities in the aligned TM domain sequences (Table 17).

The deduced amino acid sequences for Isdop1, SEQ. ID NO. 6, and Isdop2, SEQ. ID NO. 8, were analyzed to identify structural features typically associated with GPCRs and for sites predicted to undergo post-translational modification (Table 18). Each receptor contains a cysteine residue in the first and second extracellular loops, which are believed to form disulfide bonds to maintain GPCR stability (Dixon et al., 1987; Karnik et al., 1988; Fraser, 1989). There are two and four cysteines positioned near the C-terminus of Isdop1 and Isdop2, respectively, which may be suitable sites for post-translational palmitoylation and potentially contribute to the establishment of a fourth intracellular loop (Jin et al., 1999). In the N-terminal extracellular domains, two and four 1e4 N-linked glycosylation sites were predicted in Isdop1 and Isdop2, respectively, which may be needed for localization in the plasma membrane (Karpa et al., 1999). Multiple potential protein kinase A and protein kinase C phosphorylation sites were identified in the intracellular loops and carboxyl termini of each receptor (Namkung and Sibley, 2004). Isdop1 has a short third intracellular loop (47 amino acids), relative to this larger feature in Isdop2 (90 amino acids).

Each I. scapularis sequence was further examined to identify key amino acid residues which are conserved among biogenic amine receptors in the rhodopsin-like receptor subfamily of GPCRs (Table 18). Both receptor sequences contained the signature aspartate (D) residues in TMII and TMIII which are involved with binding the amine groups of catecholamines (Strader et al., 1988). Adjacent to the terminus of TMIII, Isdop2 contained the conserved “DRY” motif believed to function in coupling with G proteins (Dixon et al., 1987; Fraser et al., 1988), whereas Isdop1 included a substitution of phenylalanine (F) for tyrosine (Y) in the third position of this motif. Three conserved serines were present in TMV of each receptor that are reportedly important for hydrogen bonding with catechol hydroxyl groups (Strader et al., 1989; Pollock et al., 1992). Conserved aromatic residues were identified in TMV and TMVI of each receptor which are thought to interact with the catechol aromatic ring during ligand binding (Strader et al., 1995).

### Gene Expression

We were unable to identify ESTs in GenBank corresponding to Isdop1 or Isdop2, which necessitated gene expression analyses to determine if mRNA transcripts were produced for these receptors in vivo. We were successful in detecting transcripts for both of the single exon genes Isdop1 and Isdop2 using a 30-RACE method, as analyzed by gel electrophoresis. For Isdop1, a single 30-RACE amplification product (1050 bp) was produced and cloned. A consensus sequence was generated that, after the reverse primer sequence was removed, had >99% nucleotide identity (998/1002 bp) to the 30 end of the ISCW001496 gene sequence from the I. scapularis genome assembly. This sequence included a 107 bp region upstream of the final position in the predicted stop codon and then extended 894 bp downstream into the presumed 30 untranslated region (UTR). A putative polyadenylation site (AATAAA) for Isdop1 was identified in the 30-RACE amplified region 862 bp downstream of the final base pair position of the predicted stop codon.

For Isdop2, two prevalent (704 bp and 497 bp) and three less abundant (w1.2 kb, w350 bp, w250 bp) 30-RACE products were produced. For purposes of confirming gene expression, we successfully cloned and sequenced the two most abundant products. The consensus sequences of these amplicons had, after the reverse primer sequences were removed, >99% (649/652 bp) and >99% (448/451 bp) nucleotide identities to the corresponding 30 end of the ISCW008775 gene sequence from the I. scapularis genome assembly. The 50 region of these amplification products corresponded to the same position where the forward primers were designed, 70 bp upstream from the final base pair position of the predicted stop codon. Thereafter, these overlapping sequences differed in length as they extended into the presumed 30-UTR region 380 bp and 580 bp downstream of the final base pair position of the predicted stop codon, respectively. A putative polyadenylation site (AATAAA) was identified in the 30-RACE amplified region 358 bp downstream of the final base pair position in the predicted stop codon.

### Pharmacological Characterization

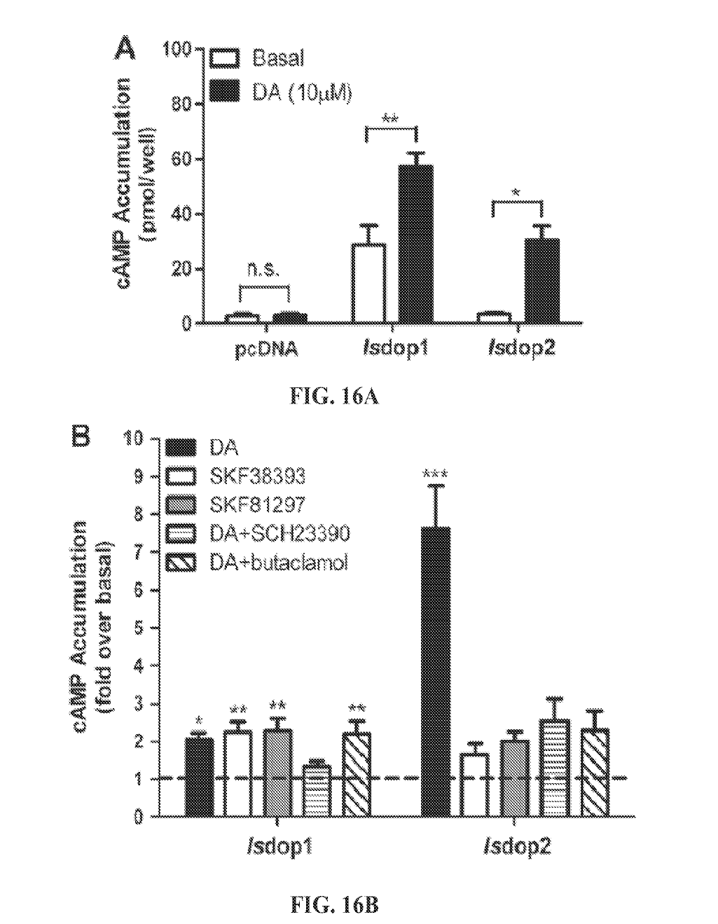

To investigate the pharmacological characteristics of Isdop1 and Isdop2, each cloned receptor was transiently-expressed in HEK 293 cells, and responses to drug treatments were measured in terms of cAMP accumulation. Dopamine treatment (10 μM) of cells expressing these receptors resulted in a significant increase of intracellular cAMP (FIG. 16A). Relative to basal levels, the increase of cAMP was approximately two-fold in cells expressing Isdop1 and nine-fold for Isdop2. Isdop1 exhibited higher constitutive activity than Isdop2, as indicated by the elevated levels of intracellular cAMP detected in Isdop1-expressing cells in the absence of dopamine. To examine the specificity of Isdop1 and Isdop2, cAMP accumulation assays were conducted in response to a series of biogenic amines (Table 19). Dopamine caused a significant increase in cAMP levels in cells expressing Isdop1 (p<0.01) and Isdop2 (p<0.001). It was observed that both epinephrine and norepinephrine caused a slight increase in cAMP levels in cells expressing these receptors; this was significant in Isdop1 cells (p<0.05) only (Table 19).

To further characterize the pharmacological profiles of Isdop1 and Isdop2, treatments with receptor agonists and antagonists were conducted (FIG. 16B). For Isdop1, treatment with the vertebrate D1-like dopamine receptor agonists SKF38393 and SKF81297 each caused modest, yet significant increases in levels of intracellular cAMP compared to basal conditions, which were similar to that observed for dopamine treatment. In contrast, only a slight increase in cAMP (ca. two-fold-over-basal) was detected for treatment with these agonists in cells expressing Isdop2, which was markedly less than the response observed for treatment with dopamine. For Isdop1, the D1-selective antagonist SCH23390 caused a significant reduction in levels of intracellular cAMP that were not significantly different from basal conditions, indicative of inhibition, whereas butaclamol did not cause a similar response. For Isdop2, treatment with dopamine in combination with either antagonist resulted in cAMP levels not significantly different from that detected in the basal measurement, consistent with an inhibition of the dopamine stimulated responses.

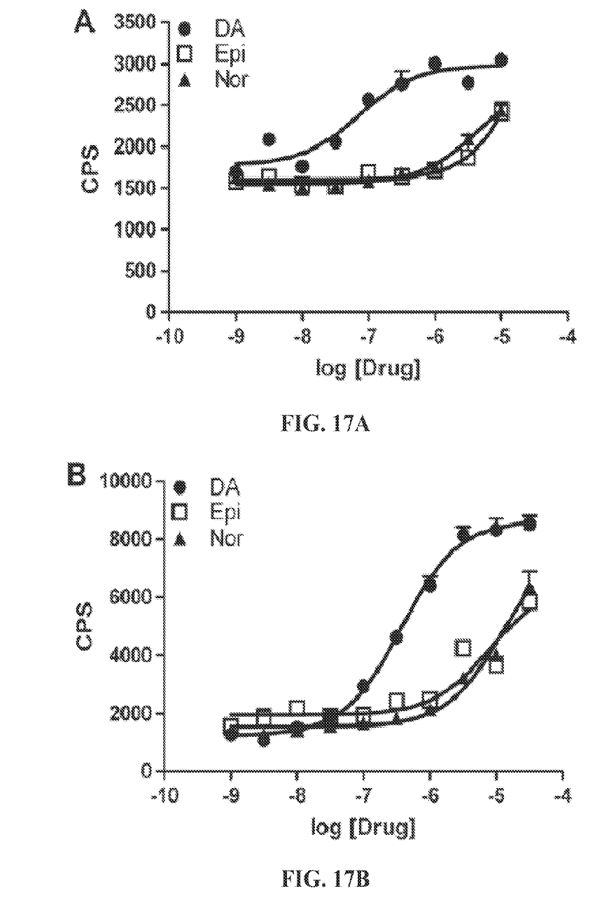

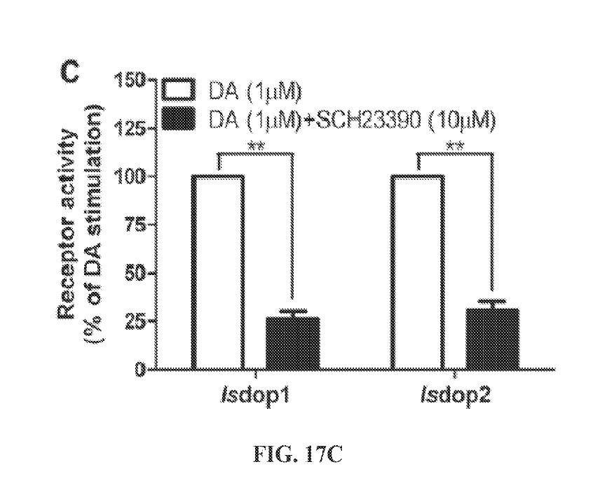

Clones stably expressing Isdop1 and Isdop2 were developed and used in conjunction with a CRE-Luc reporter assay to facilitate functional activity measurements of these receptors in a 384-well format (FIGS. 17A and 17B). With this cellular system, dose-response curves reflected the higher constitutive activity for Isdop1 relative to Isdop2 that was also observed in the cAMP accumulation experiments. To further examine the stimulatory responses observed for epinephrine and norepinephrine in the transiently-expressing cells (see Table 19), we also investigated this using our stable cell lines. The EC50 values determined for dopamine were 31_{—}5 nM for Isdop1 and 145_{—}21 nM for Isdop2 (n=16). In contrast, each of the estimated EC50 values for epinephrine and norepinephrine were markedly higher (FIGS. 17A and 17B). Specifically, for Isdop1, the estimated EC50 values for epinephrine and norepinephrine were 1.9±0.3 μM and 2.2±0.3 μM (n ¼ 5), respectively. For Isdop2, the EC50 value was 3.5±1.0 μM for epinephrine and 4.3±1.3 μM for norepinephrine (n=5). These results further support that Isdop1 and Isdop2 are dopamine receptors, with only low sensitivity to other biogenic amines.

Next, we investigated the effect of co-treatment with the D1-like dopamine receptor antagonist SCH23390 in the CRE-Luc reporter system. Relative to stimulation with dopamine alone, co-treatment with SCH23390 caused a 74% and 69% inhibition of luciferase activity for Isdop1 and Isdop2, respectively (FIG. 17C). Furthermore, when the Isdop1-expressing cells were treated with SCH23390 alone, inhibition of the constitutive activity of Isdop1 was observed, suggesting that SCH23390 may also function as an inverse agonist in this cellular model.

### Discussion

The biogenic amine receptors represent candidate neuroactive targets for the identification of GPCR-specific chemistries potentially useful for tick control (Lees and Bowman, 2007). This study provided the first pharmacological characterization of two cloned dopamine receptors in the Lyme disease vector, I. scapularis. Previous research indicated that the dopaminergic pathway is a critical component of the salivary secretion mechanisms in ticks, which are stimulated during their bloodfeeding behavior (Sauer et al., 2000; Bowman and Sauer, 2004; Lees and Bowman, 2007). Thus, improvements in our knowledge of these complex processes may open new doors toward understanding the dynamics of blood-borne pathogen acquisition and transmission among species in this medically-important arthropod lineage.

Tick genome sequencing projects provide a means to expedite determinations of gene function (Van Zee et al., 2007). The two D1-like dopamine receptor sequences analyzed here, along with a single uncharacterized D2-like receptor identified in the genome assembly, are believed to compose the primary suite of receptors which regulate dopaminergic processes in I. scapularis. Interestingly, Isdop1 and Isdop2 are single exon genes, which contrasted with that reported for other invertebrate D1-like dopamine receptors that contain introns (Mustard et al., 2005), but corresponded with the intronless human D1-like dopamine receptors (Sunahara et al., 1990, 1991). The chromosomal positions of Isdop1 and Isdop2 are unknown because, to date, chromosomal mapping in I. scapularis includes only a preliminary linkage map (Ullmann et al., 2002) and a fluorescent in situ hybridization-based map of the major tandem repeats in the genome (Meyer et al., 2010). The 30-RACE experiments demonstrated that both Isdop1 and Isdop2 were expressed in adult female ticks, supporting their functionality in vivo. Further analyses of gene expression involving different life stages and specific tissues, as well as supporting investigations of the 50- and 30-untranslated regions, would improve our understanding of tick dopamine receptor biology. During the review process of this manuscript, a relevant study by _Simo et al. (2011) provided evidence that an I. scapularis D1 receptor (a.k.a. Isdop1) was positioned on the luminal surface of cells lining the salivary gland acini and activated by a paracrine signal of dopamine.

The previously characterized D1-like dopamine receptors in the model insects D. melanogaster and A. mellifera were used to predict orthologous relationships in I. scapularis and to identify conserved structural features of the tick receptors. A neighbor joining analysis of the Isdop1 and Isdop2 amino acid sequences separated the tick receptors into two different clades, with each Glade containing an orthologous receptor from the model insects used in the analysis. Each of these clades was separate from the Glade containing the two human D1-like dopamine receptors. Furthermore, this analysis showed that the Glade containing sequences for Isdop2 and its insect orthologs also included the octopamine receptors OAMB (Han et al., 1998) and AmOA1 (Oar) (Grohmann et al., 2003) from D. melanogaster and A. mellifera, respectively, which further distinguished these receptors from the other D1-like dopamine receptors used in the sequence alignment (Mustard et al., 2005). Our notion that Isdop1 and Isdop2 would function as D1-like dopamine receptors was supported by the fact that each of the orthologous receptors in the model insects were previously characterized as functional D1-like dopamine receptors (reviewed by Mustard et al., 2005). Based on alignments of the conserved TM domains, only 47-53% amino acid identities were shared between either of the tick receptors and the human D1-like dopamine receptors, whereas 65-74% identities were observed between the tick receptors and their putative orthologs in the model insects. Together, the sequence analyses provided evidence to suggest that the I. scapularis proteins would likely have different pharmacological characteristics from each other, as well as from the human and orthologous insect D1-like dopamine receptors.

The pharmacological properties of Isdop1 and Isdop2 were compared through heterologous expression experiments in HEK 293 cells. Both Isdop1 and Isdop2 were stimulated with dopamine treatment, as determined by the subsequent accumulation of intracellular cAMP; however, each receptor had a unique pharmacological profile. We detected (FIG. 16A) an approximately two-fold increase in cAMP accumulation relative to basal levels in dopamine-treated cells transiently expressing Isdop1, whereas this response was approximately nine-fold for Isdop2. Likewise, a greater response to dopamine treatment was observed in DopR99B (DAMB) in comparison to D-Dop1 of D. melanogaster (Gotzes et al., 1994; Han et al., 1996). Initial functional studies of the Isdop1 and Isdop2 receptors revealed differences in constitutive activity in transiently transfected cells when analyzed for cAMP accumulation. This was further substantiated by characterization of the dopamine response in stably-expressing cell lines using a CREluciferase reporter assay, where Isdop1 also displayed a higher constitutive activity and had a slightly increased sensitivity to dopamine (EC50 ¼ 31_{—}5 nM) when compared to Isdop2 (EC50 ¼ 145_{—}21 nM). These receptor responses to dopamine treatment were comparable to that observed for D-Dop1 of D. melanogaster also expressed in HEK 293 cells (EC50 ¼ 500 nM) (Gotzes et al., 1994). Interestingly, the differences between the two Gas-coupled dopamine receptors in I. scapularis mirror the observations reported for the human D1-like receptors, where the human D5 receptor displays a higher constitutive activity and lower EC50 value for dopamine when compared to the human D1 receptor (Tiberi and Caron, 1994). Such constitutive activity was also reported for the A. mellifera D1-like dopamine receptor DOP1 (Mustard et al., 2003) and the nematode Caenorhabditis elegans D1-like dopamine receptor CeDOP1 (Sanyal et al., 2004). At present, we do not have evidence supporting the constitutive activity of Isdop1 in vivo, but this has been documented for other GPCRs, including those which bind neurotransmitters functioning to maintain basal neuronal activity (Seifert and Wenzel-Seifert, 2002). Interestingly, Isdop1 included a substitution of the “DRY” motif believed to interact with G proteins, where the hydrophobic residue phenylalanine (F) was substituted for the typical polar amino acid tyrosine (Y) in the third position of this motif Such a substitution also was reported in the human D4 dopamine receptor (Van Tol et al., 1991). There is evidence to suggest that this substitution may have an impact on the constitutive activity observed for Isdop1. For example in the crustacean Panulirus interruptus, a 5-HT2 receptor containing such a “DRF” motif was shown to have constitutive activity that could be reduced following mutagenic restoration to the evolutionary conserved “DRY” motif. However, the “DRY” motif is present in the aforementioned constitutively-expressing D1-like dopamine receptors in A. mellifera, H. sapiens, and C. elegans, indicating involvement of additional factors. Site-directed mutagenesis experiments may shed light on the hypothesis that this substitution contributes to the constitutive activity observed for Isdop1.

Additional pharmacological differences between Isdop1 and Isdop2 were suggested in studies examining the specificity of these receptors in response to seven different biogenic amines, where Isdop1 was activated by dopamine, epinephrine, and norepinephrine, but Isdop2 only significantly responded to dopamine treatment. The high constitutive activity of Isdop1 made determining the effect of these biogenic amines somewhat challenging, whereas for Isdop2, dopamine was clearly the most robust stimulatory ligand among the biogenic amines tested. The significant response of cells transiently expressing Isdop1 to epinephrine and norepinephrine presumably reflects the lack of absolute receptor specificity to these biogenic amines at the upper ranges of concentrations tested (i.e. 10 μM). Subsequent dose-response studies revealed that the EC50 values were markedly higher for epinephrine and norepinephrine compared to dopamine for both Isdop1 and Isdop2. In terms of determining the suitability of these receptors for future high-throughput screening assays for agonists, it appears that Isdop2 is a better candidate than Isdop1 because of its strong response to stimulation and lack of detectable constitutive activity.

The I. scapularis receptors showed differential activities in their responses to D1-like dopamine receptor agonists, where Isdop1 was significantly more responsive than Isdop2. In spite of the predicted orthology of Isdop1 with the receptors in D. melanogaster and A. mellifera, divergences in their pharmacological profiles were observed with regard to agonist treatments. For example, stimulation of D-Dop1 from D. melanogaster with SKF38393 was 3-fold less than that observed for dopamine treatment, and SKF81927 also had relatively poor stimulatory effects (Gotzes et al., 1994; Sugamori et al., 1995). This also corresponded with that reported for DOP1 of A. mellifera, where SKF38393 was not an effective agonist (Blenau et al., 1998). In contrast to Isdop1, similar trends regarding agonist activities were observed between Isdop2 and its insect orthologs. For example, SKF38393 was only a weak agonist of DopR99B (DAMB) of D. melanogaster (Feng et al., 1996) and also failed to significantly stimulate DOP2 of A. mellifera (Mustard et al., 2003).

Inhibition of the dopamine response with antagonist treatment of cells transiently expressing either Isdop1 or Isdop2 also provided evidence highlighting the distinct pharmacological properties of these receptors and permitted functional comparisons between their orthologs in D. melanogaster and A. mellifera. SCH23390 was an effective antagonist of Isdop1, whereas butaclamol did not appear to have a considerable inhibitory effect. This was in contrast to that reported for the orthologous D-Dop1 receptor in D. melanogaster and the DOP1 receptor in A. mellifera, where receptor activities were significantly reduced with butaclamol, while SCH23390 was less effective (Gotzes et al., 1994; Blenau et al., 1998). For Isdop2, both butaclamol and SCH23390 had antagonistic effects. This corresponded with that reported for DopR99B (DAMB) of D. melanogaster, where modest inhibition was shown for both SCH23390 and butaclamol (Han et al., 1996; Feng et al., 1996). Similarly, each of these antagonists also significantly reduced cAMP levels in cells expressing DOP2 of A. mellifera, relative to the response observed following treatment with dopamine (Mustard et al., 2003). The antagonistic effect of SCH23390 observed in the stable cell lines expressing Isdop1 or Isdop2 was consistent with that in our transient expression experiments.

Further pharmacological studies of the two I. scapularis D1-like dopamine receptors, as well as the uncharacterized D2-like dopamine receptor, are warranted to better understand the basic properties of these receptors. In addition to stimulation of cAMP production by adenylyl cyclase in vivo, it will also be important to determine if the tick dopamine receptors activate Ca2 [symbol] signaling pathways, as was shown for DopR99B (DAMB) of D. melanogaster (Reale et al., 1997; Feng et al., 1996). Our production of stable cell lines expressing Isdop1 and Isdop2 provided the foundation for ongoing efforts to screen chemical libraries to further characterize the pharmacology of these receptors. The significant inhibition of luciferase activity observed following treatment with the antagonist SCH23390 in cells stably expressing either Isdop1 or Isdop2 demonstrated the utility of the reporter system to readily measure the effect of antagonistic chemistries in a high-throughput format. In addition, inhibition of the constitutive activity of Isdop1 by SCH23390 indicated that this system may also be useful in screening for inverse agonists. Additional research is needed to determine if chemistries can be designed that specifically target these tick receptors but do not influence the dopaminergic processes in other organisms. This line of research in I. scapularis may serve as a model for complementary studies in other ticks for which chemical control is a primary means of population suppression for tick-borne disease management

### Materials and Methods

### Cells and CRELuc Assay

Receptor activation was detected using a CRELuc luciferase reporter assay in which the luciferase (Luc) reporter gene (pGL3, Promega, Madison, Wis.) is under transcriptional control of five copies of the cAMP response element (CRE). Dopamine-stimulated activation of the receptor results in an accumulation of intracellular Camp leading to an increase in luminescence measured in counts per second (CPS). To study a uniform population of cells expressing the receptors, stable cell lines in HEK293 cells were established and maintained as previously described (Meyer et al., 2011). Cells were trypsinized, resuspended in cell culture media, pelleted by centrifugation for 5 min at 100 g, and resuspended in Opti-MEM media (Invitrogen, Carlsbad, Calif.) before being transferred to white 384-well plates (Nunc, Thermo Fischer Scientific, Rochester, N.Y.) using amultichannel pipette. Cells were plated at a final cell concentration of approximately 35,000 cells in 20 ml per well and incubated overnight in a humidified cell culture incubator (37_C with 5% CO₂). CRELuc assays (for FIG. 1) were carried out as previously described (Meyer et al., 2011) and data analysis was conducted using GraphPad Prism v.5 software (GraphPad Software Inc., San Diego, Calif.).

### Assay Validation and LOPAC1280 Library Screening

Screening of the LOPAC1280 library (Sigma, St. Louis, Mo.) for Isdop2 receptor antagonists was executed at the Integrated Screening Technologies Laboratory, Discovery Park at Purdue University, West Lafayette, Ind. Approximately 35,000 Isdop2-expressing cells (20 ml/well) were plated in solid white 384-well plates (Nunc, Thermo Fisher Scientific, Rochester, N.Y.) using a BiomekFX instrument (Beckman-Coulter, Brea, Calif.), incubated overnight and the screening assays were carried out as previously described (Meyer et al., 2012) with the exception that fresh cells (in contrast to cryopreserved cells) were used. Briefly, library compounds were added to the cells followed by addition of dopamine, and the assay plates were incubated for 2 h (37_C with 5% CO2). The plates were equilibrated to room temperature, and the reaction was terminated following addition of 10 μl/well Steadylite Plus reagent (PerkinElmer, Waltham, Mass.). The dopamine stimulated response was determined by measuring luminescence on a DTX880 multimode reader (Beckman Coulter, Brea, Calif.) with a 1 s integration time. To evaluate the robustness of the screening protocol, the Z factor (Zhang et al., 1999) was determined for the assay platform as follows. Plates of Isdop2-expressing cells were prepared with alternating wells containing either dopamine (3 μM) or dopamine (3 μM) in combination with the dopamine receptor antagonist SCH23390 (10 μM) in a “checkerboard” pattern. Data from the assay plates were analyzed to calculate the Z0 using a modification of the original equation (Zhang et al., 1999) that considers the number of replicate samples in the screening assay protocol [http://assay.nih.gov/assay/index.php/Section2:Plate_Uniformity_and_Signal_Variability_Assessment]. Library plates were thawed, and aliquots were diluted in assay buffer using a BiomekFX liquid handling station (Beckman-Coulter, Brea, Calif.). Each library compound was screened in quadruplicate, with duplicates of each compound dispensed on separate plates as a control for potential plate-specific effects. Compounds were tested at a final concentration of 10 μM in combination with dopamine (3 μM). Each plate included samples of dopamine (3 μM) as a positive control for receptor activation, and dopamine (3 μM) in combination with SCH23390 (10 μM) as a control for antagonist chemistries (Meyer et al., 2011). A dopamine concentration-response curve (0.14-300 μM DA) was also included for each plate to enable comparison of data between plates. To analyze screen data, the average background luminescence for each plate was subtracted from all values on the corresponding plate. Wells containing dopamine only (no test compound) were used to establish the dopaminestimulated response control value for each plate. The percent inhibition of the dopamine-stimulated response associated with each test compound was calculated by averaging the four replicate values for each compound and normalizing them compared to the response associated with dopamine alone. These values were then compared to the inhibitory effect of the antagonist control (SCH23390) averaged between all 16 plates. Compounds were considered “hits” if their % inhibition value was three standard deviations below the % inhibition value recorded for SCH23390. In the present study the average_S.D. inhibitory value of SCH23390 was calculated as 86±6%. This value minus three standard deviations (i.e. 86%−(3×6%) ¼ 68%) was used to establish the cutoff for identification of hit compounds.

### Confirmation Assays

Screen validation analysis was conducted with cells stably expressing Isdop1 or Isdop2 (Meyer et al., 2011) using a cAMP accumulation assay as previously described (Meyer et al., 2012) with minor modifications. Briefly, cryopreserved cells were thawed and resuspended in HBSS-based assay buffer (Hank's Balanced Salt Solution (Hyclone, Logan, Utah) supplemented with 0.1% bovine serum albumin (BSA) (Sigma, St. Louis, Mo.), 20 mM 4-(2-Hydroxyethyl) piperazine-1-ethanesulfonic acid N-(2-Hydroxyethyl)piperazine-N0-(2-ethanesulfonic acid) (HEPES, Sigma, St. Louis, Mo.) and 0.02% ascorbic acid), counted, and seeded (30 ml/well) in clear tissue culture treated 96 well plates (BD Falcon, Franklin Lakes, N.J.). To obtain optimal signals in the cAMP accumulation assay, 50,000 cells (Isdop1) or 100,000 cells (Isdop2) were seeded and allowed to recover for 6 h (Isdop1) or 1 h (Isdop2) in a cell culture incubator before proceeding with the assay. Compounds evaluated in confirmation assays were amitriptyline hydrochloride, ( [symbol] )-butaclamol hydrochloride, cis-(Z)-flupenthixol dihydrochloride, clozapine, doxepin hydrochloride, mianserin hydrochloride (Sigma, St. Louis, Mo.), and methiothepin maleate and R(+)—SCH23390 (R(+)-7-Chloro-8-hydroxy-3-methyl-1-phenyl-2,3,4,5-tetrahydro-1H-3-benzazepine) hydrochloride (Tocris, Ellisville, Mo.). The antagonist hit compounds were prepared as DMSO stocks and then diluted in HBSS assay buffer supplemented with 3-isobutyl-1-methylxanthine (IBMX, final assay concentration 0.5 mM, Sigma, St. Louis, Mo.). Serial dilutions were carried out with a Precision 2000 liquid handling station (BioTek, Winooski, Vt.). Diluted antagonists were added and plates were incubated at room temperature for 10 min followed by addition of dopamine at concentrations corresponding to the approximate EC90 values for Isdop1 (10 nM) and Isdop2 (30 μM) as determined in the cAMP accumulation assay. Assays were carried out in duplicate and data analysis was conducted with GraphPad Prism v.5 software, constraining the top of the curves to 100 for both receptors and the Hill slope to _{—}0.7 for Isdop2.

### Results and Discussion

### Assay Establishment for High Throughput Screening

Two Gas-coupled dopamine receptors (Isdop1 and Isdop2) with distinct molecular and pharmacological profiles have been identified in the Lyme disease tick, I. scapularis (Meyer et al., 2011; _Simo et al., 2011). Due to their potential as new molecular targets for tick control, we aimed to further characterize and identify novel small molecule antagonists of Isdop1 and Isdop2. Our data in HEK293 cells support the notion that both Isdop1 and Isdop2 are Gas coupled, however, our observations do not exclude that these receptors may also signal through other pathways, which were not examined here. The Isdop2 receptor displays a lower constitutive activity and higher fold-stimulation in response to dopamine than Isdop1 (approximately 9-fold vs. 2-fold) when heterologously expressed in HEK293 cells (FIG. 1). Furthermore, Isdop2 exhibits a modestly reduced sensitivity to dopamine compared to Isdop1, with EC50 values for Isdop1 and Isdop2 of approximately 31 and 145 nM, respectively (Meyer et al., 2011). The presence of two pharmacologically distinct Gas-coupled dopamine receptors in I. scapularis is consistent with that in mammalian and other arthropod systems. For instance, the respective Gas-coupled dopamine receptors D1 and D5 in humans (Sunahara et al., 1991, 1990), DmDOP1 and DAMB/DopR99B in Drosophila melanogaster (Feng et al., 1996; Gotzes et al., 1994) and AaDOP1 and AaDOP2 in A. aegypti (Meyer et al., 2012) differ in both intrinsic activity and apparent affinity for dopamine.

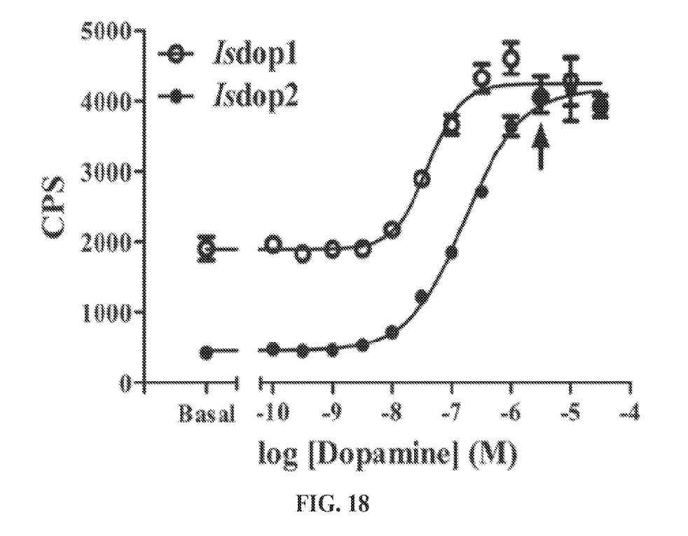

Because antagonists of the AaDOP2 receptor showed toxicity to mosquito larvae (Meyer et al., 2012), we reasoned that the tick dopamine receptors may represent a new class of drug targets for acaricide development. To screen for novel antagonists of the Ixodes dopamine receptors, we developed a luciferase reporter-based screening platform using cells that stably express the Isdop2 receptor in conjunction with the CRELuc luciferase reporter construct (Meyer et al., 2011) to measure dopamine receptor activation. The CRELuc reporter assay is based on an indirect measure of cAMP; receptor activation and subsequent increase in cAMP leads to phosphorylation of CREB causing activation of the luciferase reporter gene that is under transcriptional control of the cAMP response element (CRE). The Isdop2 receptor showed a robust response to dopamine activation and greater signal-to background window relative to Isdop1 (FIG. 18), and accordingly Isdop2 was selected for assay development, optimization, and subsequent chemical library screen for receptor antagonists. Assay validation analysis was conducted using the maximal and minimal screening conditions in a checkerboard pattern to assess the robustness and utility of the assay platform and its parameters (e.g. cell number, dopamine and SCH23390 concentrations, and instrumentation). A concentration of 3 μM dopamine (the approximate EC90 value for Isdop2, labeled with an arrow in FIG. 18) was used to define the maximal signal value. We have previously shown that SCH23390 is an antagonist of dopamine-stimulated activity of both Isdop1 and Isdop2 (Meyer et al., 2011); thus, 3 μM dopamine in combination with the D1 receptor antagonist SCH23390 (10 μM) was used to define the minimum signal value. Using four replicate samples, the specific Z′ for the Isdop2 antagonist assay platform was 0.55_{—}0.05 (n ¼ 4, average S.E.M.). As Z′ values greater than 0.5 indicate high assay performance (Iversen et al., 2006; Zhang et al., 1999), this analysis enabled the validation of the Isdop2 assay platform for the antagonist screen.

### Screening the LOPAC1280 for Isdop2 Receptor Antagonists

To the best of our knowledge, the Isdop2 antagonist screen described herein is the first chemical library screen published for any tick GPCR. The average inhibitory effect of 10 mM SCH23390 (assay antagonist control) across all sixteen library compound plates was calculated as 86±6%. This value minus three standard deviations (i.e. 68%) was used to establish the cutoff for identification of hit compounds. Thus, compounds that resulted in an average inhibition of >68% of the dopamine-stimulated signal were reported as hits. Using these criteria, 85 hit compounds were identified in the Isdop2 antagonist screen, corresponding to a 7% hit rate (Table 20). The demonstrated pharmacological activities of the compounds in the LOPAC1280 small molecule collection, coupled with the composition of this library (>50% of library chemistries target GPCRs, including dopamine receptors) made the LOPAC1280 a suitable choice for screen validation and Isdop2 antagonist discovery. Use of this library also allowed comparisons to a similar screening effort directed at discovery of novel AaDOP2 antagonists (Meyer et al., 2012). Of the 85 hit compounds discovered here, a large fraction includes GPCR ligands with reported activity in mammalian systems. Mammalian dopamine receptor ligands (primarily benzazepines, phenothiazines, and thioxanthenes) constituted the largest class of hit compounds (26 compounds; 31% of hits). Interestingly, this class also included several known dopamine receptor agonists, as well as chemistries selective at Gai coupled dopamine receptors, suggesting a divergent pharmacological profile of Isdop2 compared to mammalian Gas-coupled dopamine receptors. Other major classes of hits included biogenic amine reuptake inhibitors (9 compounds; 11% of hits) and serotonin receptor ligands (9 compounds; 11% of hits), both of which contained multiple tricyclic antidepressant (TCA) derivatives. TCAs are widely used in medicine, and the observation that several TCAs act on the tick dopamine receptor is not entirely surprising because a number of TCAs have reported affinities for mammalian dopamine receptors at modest nanomolar concentrations. Another large group was comprised of protein kinase modulators (14 compounds; 16% of hits). The abundance of structurally diverse kinase modulators among the hits likely reflects off-target effects, rather than specific effects on the Isdop2 receptor, because the luciferase reporter system relies on protein kinase-dependent phosphorylation of CREB. In support of this hypothesis, we previously showed that the kinase inhibitor piceatannol, which was identified as an AaDOP2 antagonist in the luciferase reporter-based screen, did not exhibit measurable activity at the AaDOP2 target in a cAMP accumulation confirmation assay (Meyer et al., 2012).

### Confirmation of Hits and Comparative Pharmacology

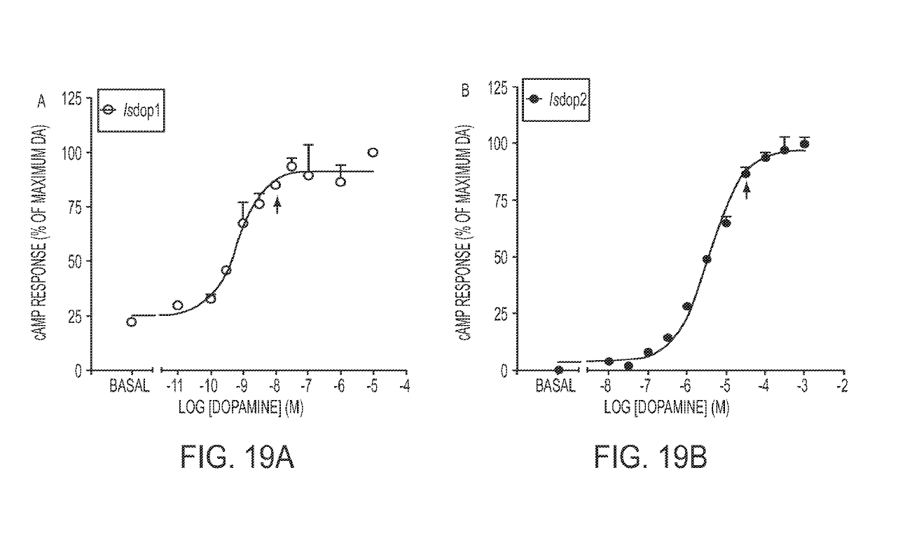

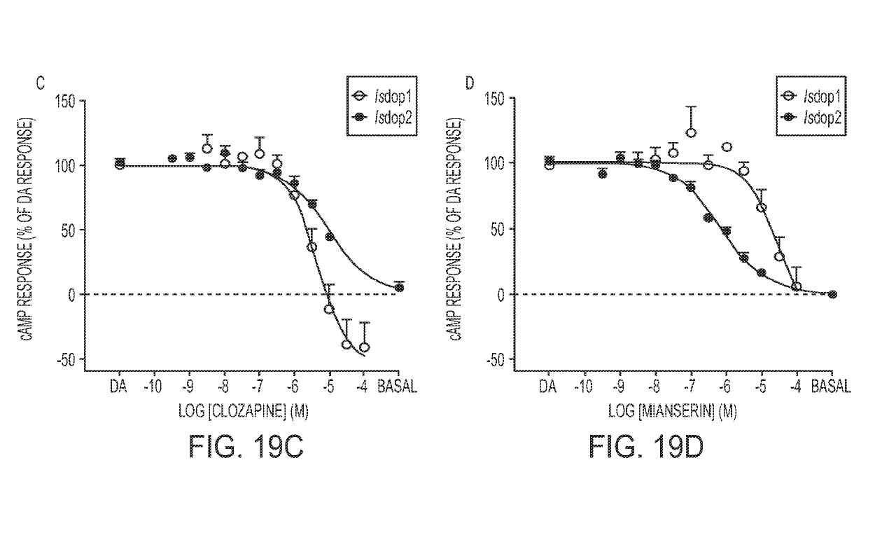

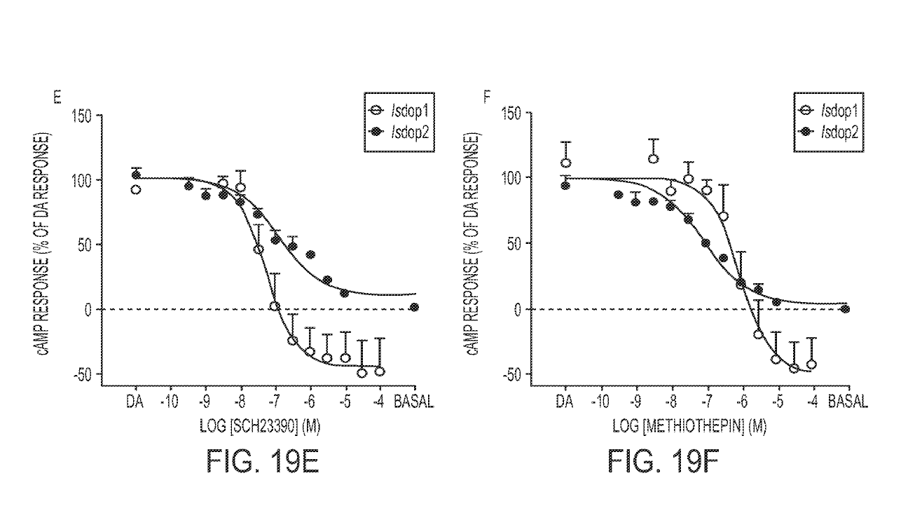

In order to study the relative potency of a subset of the hit antagonistic compounds, we conducted secondary assays that employed direct measurement of cAMP. Eight compounds belonging to three chemical classes were selected for confirmation assays. These included four compounds from the dopamine receptor ligand class (butaclamol, clozapine, cis-(Z)-flupenthixol, and SCH23390), two serotonin receptor ligands (mianserin and methiothepin) and two biogenic amine reuptake inhibitors (amitriptyline and doxepin). Each of these compounds was also shown to have antagonist properties at the AaDOP2 receptor from the mosquito A. aegypti (Meyer et al., 2012). The effects of antagonist hits on the dopamine-stimulated cAMP accumulation of both Isdop1 and Isdop2 were evaluated in order to compare the pharmacological profiles of the receptors. All eight compounds showed inhibition of the dopamine-stimulated response of the Isdop1 and Isdop2 receptors, but considerable differences in potency and relative selectivity were observed (Table 21). Five of the eight compounds were more potent at the Isdop2 receptor (i.e. exhibited a lower IC50 value) compared to Isdop1, with the exceptions being SCH23390, doxepin, and clozapine, for which the range of IC50 values were overlapping at both receptors. An inherent difference between these two receptors is the high constitutive activity of Isdop1 compared to Isdop2, and in addition to their antagonistic properties, all eight compounds tested here also displayed inverse agonist activity at Isdop1 (FIG. 19). Similarly, an inverse agonist effect, or inhibition of constitutive activity, was observed with cis-(Z)-flupenthixol at the AmDOP1 receptor of the honeybee Apis mellifera (Mustard et al., 2003).

Our data provided an opportunity to compare two chemical library screen data sets between orthologous dopamine receptors from a tick (Table 20) and mosquito (Meyer et al., 2012). We previously reported a chemical screen of the LOPAC1280 library in which 51 candidate antagonists of the A. aegypti AaDOP2 receptor were identified (Meyer et al., 2012). Comparison of the hit chemistries identified in these screens shows an overlap of 46 compounds, and reveals 39 hits (46%) that are unique to

Isdop2, i.e. compounds that did not meet the hit selection criteria for AaDOP2 (Table 20, indicated with superscript d). Of these 39 Isdop2-specific hits, several have intermediate activity at AaDOP2. However, ten compounds (chlorpromazine, clemastine, clomipramine, cortexolone, cyclobenzaprine, cyclosporin A, cyproheptadine, CGP-74514A, PD 98,059, and Z-L-Phe chloromethyl ketone) had no or very minor effects (<25% inhibition) on the dopamine-stimulated activity of AaDOP2 (Meyer et al., 2012). Conversely, five hit compounds (JL-18, ketotifen, maprotiline, mitoxantrone, and blapachone) from the AaDOP2 screen (Meyer et al., 2012), were not revealed as Isdop2 antagonists. Interestingly, detailed examination of the screen data revealed that all of these five chemistries, representing five different chemical classes, showed an intermediate activity at the Isdop2 receptor (50e65% inhibition of the dopaminestimulated response) suggesting that they are also modest antagonists at Isdop2.

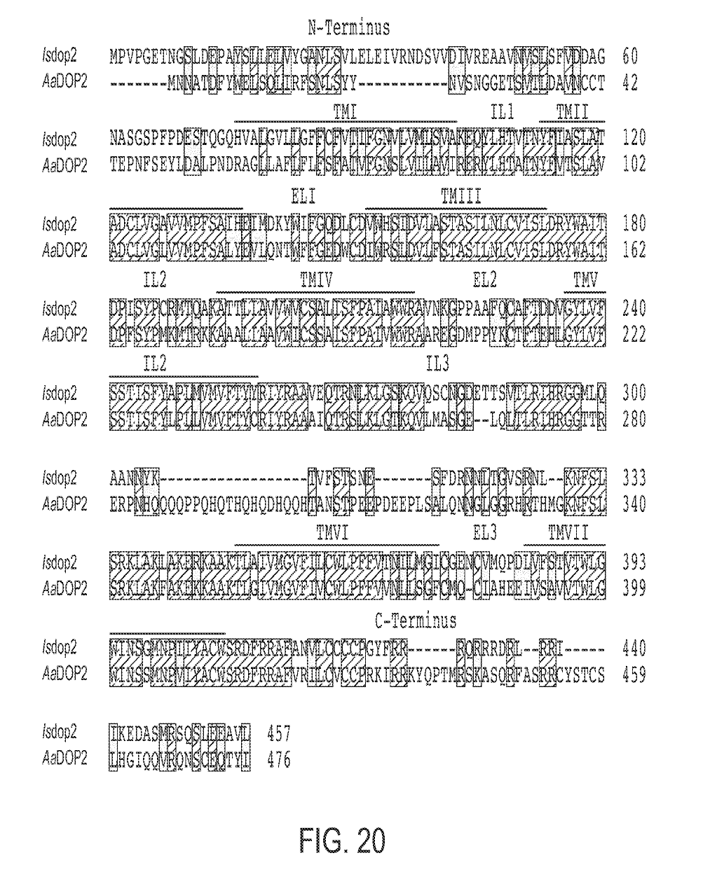

We previously reported the effects of these eight compounds (i.e. the ones selected for confirmation assays) in secondary cAMP accumulation confirmation assays at the AaDOP2 mosquito receptor and the human D1 dopamine receptor. Although each compound showed antagonistic effects on AaDOP2 and hD1, we observed important differences in the relative potencies of these compounds (Meyer et al., 2012). Together with the present data, we also noted differences in the responses to the antagonists between the orthologous Isdop2 and AaDOP2 arthropod receptors. For example, the rank order of potency for the antagonists were methiothepin>butaclamol and SCH23390>cis-(Z)-flupenthixol>amitriptyline>doxepin>clozapine compared to amitriptyline and methiothepin>cis-(Z)-flupenthixol>doxepin and clozapine>mianserin>butaclamol>SCH23390, for Isdop2 (Table 21) and AaDOP2 (Meyer et al., 2012), respectively. Notably, amitriptyline and methiothepin were the most potent AaDOP2 antagonists, whereas amitriptyline was the second and third least potent at Isdop1 and Isdop2, respectively. Additionally, whereas SCH23390 was identified as a potent antagonist at both I. scapularis receptors (Table 21) and the hD1, it had very weak antagonistic properties at the AaDOP2 receptor (approximately 3.000-fold less than that for hD1, Meyer et al., 2012). Moreover, whereas five of the eight chemistries were selective for AaDOP2 over hD1, none of the eight compounds were selective for the I. scapularis dopamine receptors over the human dopamine D1 receptor. The Isdop2 and AaDOP2 receptors share molecular characteristics, as shown by the amino acid alignment in FIG. 20. The transmembrane domains, representing the most conserved regions among GPCRs in general, are 72% identical; however these receptors are divergent in both sequence and size of the N-(Isdop2: 75 amino acids, AaDOP2: 57 amino acids) and C-termini (Isdop2: 50 amino acids, AaDOP2: 63 amino acids) as well as in the highly variable third intracellular loop (Isdop2: 90 amino acids, AaDOP2: 115 amino acids). These sequence differences may offer an opportunity to develop chemistries that specifically target individual receptors. This notion is supported by our data that show considerable differences in the pharmacology of Isdop2 and AaDOP2. The present study emphasizes the utility of confirmation assays to confirm screen hits and further explore the pharmacological nuances of each target. In addition, the confirmation assays allowed us to discriminate the relative in vitro selectivity of antagonists between different arthropod and human dopamine receptors.

Despite limited sequence similarity between the dopamine receptors of ticks, insects, and vertebrates, our approach uncovered chemistries that exhibit cross reactivity with orthologous receptors in other species, including humans. The issues of receptor selectivity and off-target effects must be rigorously evaluated. This is a primary rationale for incorporation of the orthologous human D1 receptor into our early-phase drug discovery activities (Meyer et al., 2012). Where possible, the inclusion of additional vertebrate and invertebrate receptors could also greatly enhance initial analyses of compound specificity. The LOPAC 1280 library was employed in this study as it is enriched for human GPCR active chemistries and thus well suited for screen validation. Screening the LOPAC1280 library also allowed us to identify three receptor antagonists (i.e. amitriptyline, doxepin, and clozapine) that are FDA approved and have been used in humans for decades, suggesting the potential for drug re-purposing. However, expanded screening of tens and hundreds of thousands of chemistries is required to identify novel chemical scaffolds with in vitro activity against tick dopamine targets. These efforts will explore more unique “chemical space”, thus increasing the opportunity for discovery of molecules with limited off-target effects.

### CONCLUSIONS AND FUTURE DIRECTIONS

Our development, optimization, and validation of an assay platform for chemical library screening of a tick dopamine receptor may also be adapted to screen other arthropod Gas- and Gaicoupled receptors to discover chemistries that modulate their function. Implementation of this platform and subsequent confirmation assays resulted in identification of several antagonists/inverse agonists of the two D1-like dopamine receptors in I. scapularis. The comparative molecular and pharmacological analyses between these tick receptors with that of an orthologous mosquito dopamine receptor (Meyer et al., 2012) revealed commonalities and distinct pharmacological properties, which may be further exploited in efforts to develop species-specific chemistries for vector control. This research motivates future studies aimed to investigate the in vivo activities of dopamine receptor antagonists as potential leads for acaricide discovery.

TABLE 1
Responses of the AaDOP1 and AaDOP2 receptors to biogenic
amines and synthetic dopamine receptor agonists.

EC₅₀ values

| Compound | AaDOP1 | AaDOP2 |
|---|---|---|
| Dopamine | 3.1 ± 1.1 nM | 240 ± 16 nM |
| Epinephrine | 5.8 ± 1.5 nM | ≧10 μM |
| Norepinephrine | 760 ± 180 nM | ≧10 μM |
| Histamine | ≧10 μM | ≧10 μM |
| Octopamine | ≧10 μM | ≧10 μM |
| Serotonin | ≧10 μM | ≧10 μM |
| Tyramine | ≧10 μM | ≧10 μM |
| Dihydrexidine | 6.9 ± 1.5 nM | 290 ± 54 nM |
| SKF 81297 | 24 ± 7.0 nM | ≧10 μM |
| SKF 38393 | 310 ± 46 nM | ≧10 μM |

HEK293 cells stably expressing both a CRELuc reporter construct and either of the receptors were stimulated with potential agonists. Dose-response curves were plotted and the EC₅₀ values were calculated. Compounds with EC₅₀ values ≧10 μM are considered to lack intrinsic activity at AaDOP2.

TABLE 2
Summary of antagonist hits identified from the AaDOP2 Screen again LOPAC₁₂₈₀ chemical compound
library. Additional information on screening results can be found in Tables 5-8.

| | | % of the | |
|---|---|---|---|
| | | SCH23390 | |
| AaDOP2 hit class | Chemistry | effect | Mode of action |
| Dopamine receptor | R(+)-SCH-23390 hydrochloride*^{‡} | 83 | D₁ DAR antagonist |
| antagonists (20) | (±)-Butaclamol hydrochloride^{‡} | 81 | D₂ DAR selective antagonist |
| | (+)-Butaclamol hydrochloride | 87 | DAR antagonist |
| | Chlorprothixene hydrochloride | 94 | D₂ DAR antagonist |
| | Clozapine^{‡} | 81 | D₄ DAR selective antagonist |
| | Fluphenazine dihydrochloride | 82 | DAR antagonist |
| | cis-(Z)-Flupenthixol | 88 | DAR antagonist |
| | dihydrochloride^{‡} | | |
| | JL-18 | 98 | D₄ DAR selective antagonist |
| | LE 300 | 99 | D₁ DAR antagonist |
| | Loxapine succinate | 97 | N.D. |
| | (±)-Octoclothepin maleate | 97 | D₂DAR/5-HT receptor |
| | | | antagonist |
| | Perphenazine | 95 | D₂ DAR antagonist, σ receptor |
| | | | agonist |
| | Prochlorperazine dimaleate | 83 | DAR antagonist |
| | Promazine hydrochloride | 88 | D₂ DAR antagonist |
| | Propionylpromazine | 85 | D₂ DAR antagonist |
| | hydrochloride | | |
| | Risperidone | 83 | D₂ DAR/5-HT receptor |
| | | | antagonist |
| | Triflupromazine hydrochloride | 88 | D₂ DAR antagonist |
| | Trifluoperazine dihydrochloride | 81 | DAR/calmodulin antagonist |
| | Thiothixene hydrochloride | 86 | DAR antagonist |
| | Thioridazine hydrochloride | 86 | DAR/Ca²⁺ channel antagonist |
| Serotonin receptor | Amperozide hydrochloride | 83 | 5-HT & DAR antagonist |
| ligands (6) | LY-310,762 hydrochloride | 81 | 5-HT_{1D} selective antagonist |
| | Mianserin hydrochloride^{‡} | 95 | 5-HT receptor antagonist |
| | Methiothepin mesylate^{‡} | 99 | 5-HT₁ selective antagonist |
| | Pirenperone | 90 | 5-HT₂ selective antagonist |
| | Ritanserin | 83 | 5-HT₂ selective antagonist |
| Histamine receptor | Ketotifen fumarate | 96 | H1 antagonist |
| ligands (2) | Promethazine hydrochloride | 95 | H1 antagonist |
| mAChR ligands (1) | Benztropine mesylate | 89 | mAChR antagonist |
| Biogenic amine | Amitriptyline hydrochloride^{‡} | 90 | N.D. |
| uptake inhibitors (9) | Amoxapine | 90 | NOR uptake inhibitor |
| | 4′-Chloro-3-alpha- | 85 | DA uptake inhibitor |
| | (diphenylmethoxy) tropane | | |
| | hydrochloride | | |
| | Doxepin hydrochloride^{‡} | 90 | N.D. |
| | Imipramine hydrochloride | 96 | 5-HT & NOR uptake inhibitor |
| | Maprotiline hydrochloride | 82 | NOR uptake inhibitor |
| | Nortriptyline hydrochloride | 96 | N.D. |
| | Protriptyline hydrochloride | 82 | NOR uptake inhibitor |
| | Trimipramine maleate | 87 | 5-HT & NOR uptake inhibitor |
| Protein kinase | Diacylglycerol kinase inhibitor I | 90 | Diacylglycerol kinase inhibitor |
| modulators (6) | Kenpaullone | 83 | Phosphatase inhibitor |
| | NSC 95397 | 83 | Syk, Lck inhibitor |
| | Piceatannol^{‡} | 98 | CDK inhibitor |
| | Phorbol 12-myristate 13-acetate | 88 | Activates protein kinase C |
| | Purvalanol A | 93 | CDK1, CDK2, CDK5 inhibitor |
| Miscellaneous; e.g., | beta-Lapachone | 86 | Induces apoptosis |
| cell cycle | (S)-(+)-Camptothecin | 93 | DNA topoisomerase I inhibitor |
| regulators/apoptosis | Emetine dihydrochloride hydrate | 86 | Apoptosis inducer; RNA- |
| modulators (7) | | | protein translation inhibitor |
| | Idarubicin | 83 | Disrupts topoisomerase II |
| | Mitoxantrone | 83 | DNA synthesis inhibitor |
| | Niclosamide^{‡} | 95 | Uncouples oxidative |
| | | | phosphorylation |
| | Resveratrol^{‡} | 89 | Inhibits lipo- & cyclo- |
| | | | oxygenase activity |
| Total | 51 (4% hit rate) | | |

^{†}Percent inhibition of receptor response in the presence of test compound relative to the SCH23390 control; *, SCH23390 “antagonist control”; ^{‡}, compound analyzed in cAMP confirmation assay; CDK, cyclin dependent kinase; DAR, dopamine receptor; H, histamine receptor; Lck, lymphocyte-specific protein tyrosine kinase; NOR, norepinephrine; mAChR, muscarinic acetylcholine receptor; Syk, spleen tyrosine kinase; σ, sigma receptor; 5-HT, 5-hydroxytryptamine (serotonin). N.D.=not determined.

TABLE 3
Confirmation and secondary assays for “hit” antagonist
chemistries using direct cAMP accumulation assays for Aadop2 and the
H. sapiens D₁ dopamine receptor (hD₁).

| | IC₅₀ value | IC₅₀ value | Relative fold |
|---|---|---|---|
| | (at 3 μM | (at 100 nM | selectivity |
| | dopamine for | dopamine for | for AaDOP2 |
| Compound | AaDOP2) | hD₁) | vs. hD₁ |
| Amitriptyline | 14 ± 3.4 nM | 470 ± 49 nM | 36 |
| (+) Butaclamol | 480 ± 33 nM | 3.7 ± 0.64 nM | 0.008 |
| cis-(Z)- | 20 ± 5.4 nM | 11 ± 1.9 nM | 0.55 |
| Flupenthixol | | | |
| Clozapine | 31 ± 6.5 nM | 300 ± 35 nM | 9.7 |
| Doxepin | 31 ± 4.9 nM | 960 ± 86 nM | 31 |
| Methiothepin | 14 ± 5.1 nM | 80 ± 11 nM | 5.7 |
| Mianserin | 120 ± 40 nM | 1200 ± 260 nM | 10 |
| Niclosamide | ≧10 μM | N.D. | N.D. |
| Piceatannol | ≧10 μM | N.D. | N.D. |
| Resveratrol | ≧10 μM | N.D. | N.D. |
| SCH23390 | 1600 ± 73 nM | 0.47 ± 0.03 nM | 0.0003 |

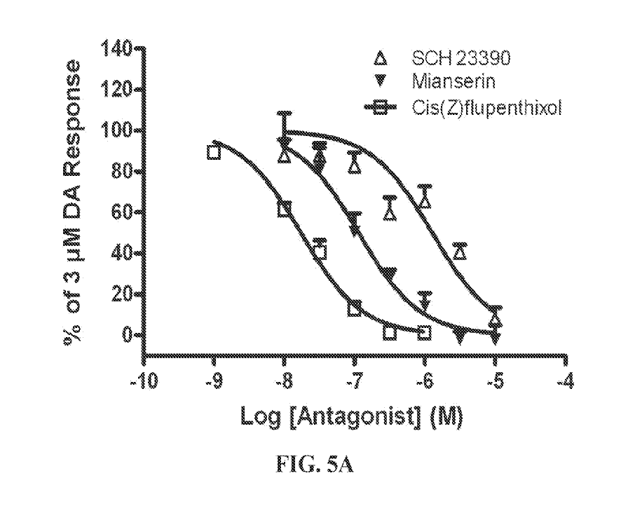

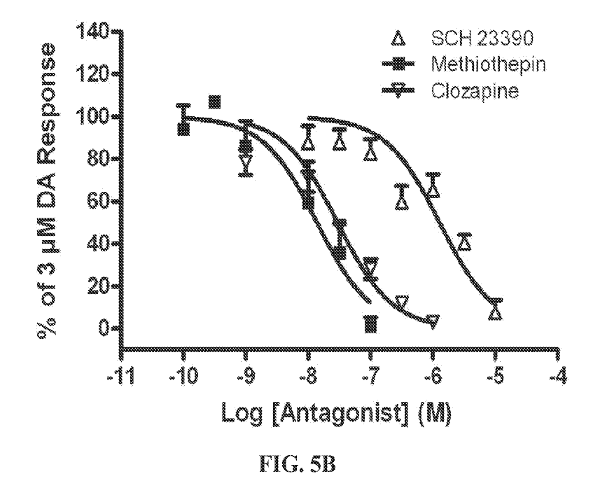

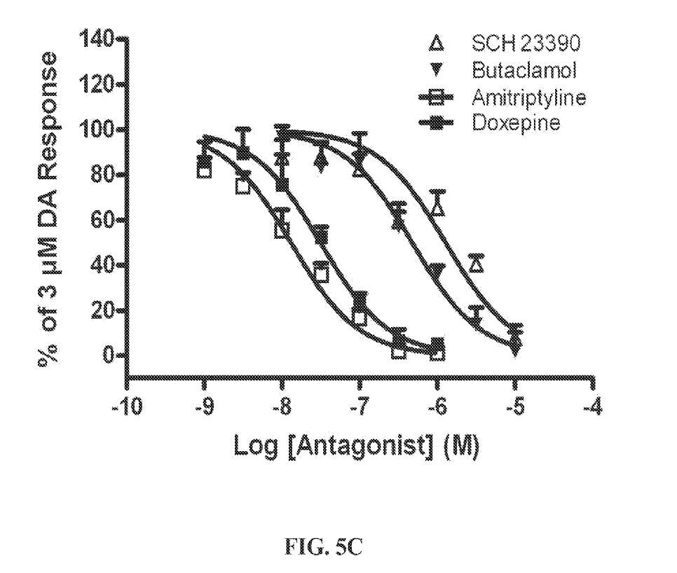

Select chemistries and the assay control (SCH23390) were tested in dose-response experiments in the presence of 3 μM dopamine in AaDOP2- or hD₁-expressing cells (FIG. 5). Compounds with IC₅₀ values ≧10 μM are considered to lack intrinsic activity at AaDOP2 and were not tested at hD₁. N.D.=not determined.

TABLE 4
A collection of some exemplary compounds that interact have an
affinity for Aedesdop2.

| | Affinity | | |
|---|---|---|---|
| Name | (Aedesdop2) | Structure | Systematic name (IUPAC) |
| Meththiothepin | 14 nM | 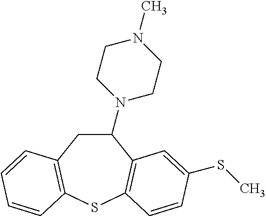 [SMILES: CSc1ccc2c(c1)C(N1CCN(C)CC1)Cc1ccccc1S2] | (1-methyl-4-(8-(methylthio)- 10,11-dihydrodibenzo- [b,f]thiepin-10-yl)piperazines) |
| Amitriptyline | 14 nM | 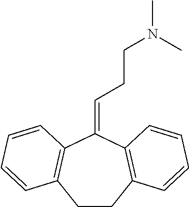 [SMILES: CN(C)CCC=C1c2ccccc2CCc2ccccc21] | (3-(10,11-dihydro-5H- dibenzo[a,d][7]annulen-5- ylidene)-N,N-dimethylpropan-1- amine) |
| Flupentixol | 20 nM | 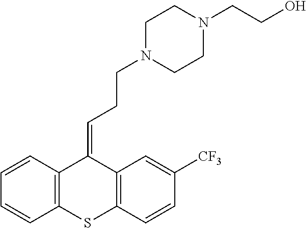 [SMILES: OCCN1CCN(CC/C=C2/c3ccccc3Sc3ccc(C(F)(F)F)cc32)CC1] | ((Z)-2-(4-(3-(2-(trifluoromethyl)- 9H-thioxanthen-9- ylidene)propyl)piperazin-1- yl)ethanol) |
| Doxepin | 31 nM | 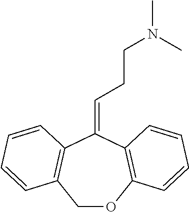 [SMILES: CN(C)CC/C=C1/c2ccccc2COc2ccccc21] | ((Z)-3-(dibenzo[b,e]oxepin- 11(6H)-ylidene)-N,N- dimethylpropan-1-amine) |
| Clozapine | 31 nM | 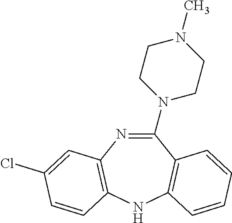 [SMILES: CN1CCN(C2=Nc3cc(Cl)ccc3Nc3ccccc32)CC1] | (8-chloro-11-(4-methylpiperazin- 1-yl)-5I-dibenzo[b,e][1,4]- diazepine) |
| Mianserin | 120 nM | 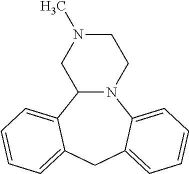 [SMILES: CN1CCN2c3ccccc3Cc3ccccc3C2C1] | ((±)-2-methyl-1,2,3,4,10,14b- hexahydrodibenzo[c,f]pyrazino [1,2-a]azepine) |
| Other related | % | Structure of | IUPAC name |
| chemical hits | inhibition of | core | |
| (from Aedes | DA activity | compound | |
| cell-based | | | |
| assay screen) | | | |
| SCH-23390 | 83 | 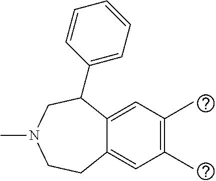 | 8-chloro-3-methyl-5-phenyl- 1,2,4,5-tetrahydro-3-benzazepin- 7-ol |
| Butaclamol | 87 | 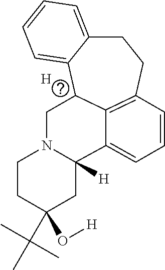 | 3-(1,1-dimethylethyl)- 2,3,4,4a,8,9,13b,14-octahydro- 1H-benzo[6,7]cyclohepta[1,2,3- de]pyrido[2,1-a]isoquinolin-3-ol |
| LE 300 | 99 | 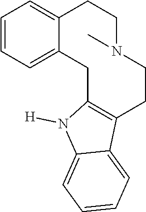 [SMILES: CN1CCc2ccccc2Cc2[nH]c3ccccc3c2CC1] | 7-Methyl-6,7,8,9,14,15- hexahydro-5H- benz[d]indolo[2,3-g]azecine |
| LY-310, 762 | 81 | 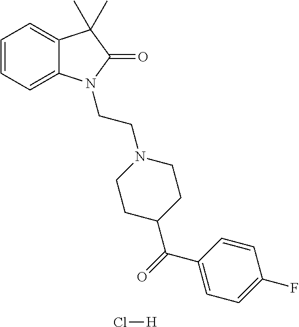 | 1-[2-[4-(4- fluorobenzoyl)piperidin-1- yl]ethyl]-3,3-dimethylindol-2- one |
| Chlorprothixene | 94 | 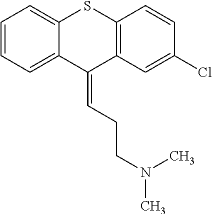 [SMILES: CN(C)CC/C=C1/c2ccccc2Sc2ccc(Cl)cc21] | (±)-2-methyl-1,2,3,4,10,14b- hexahydrodibenzo[c,f]pyrazino [1,2-a]azepine |
| Fluphenazine | 82 | 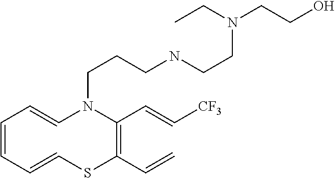 [SMILES: C=C/C1=C(\C=C\C(F)(F)F)N(CCCNCCN(CC)CCO)/C=C/C=C\C=C\S1] | 2-[4-[3-[2-(trifluoromethyl)-10H- phenothiazin-10- yl]propyl]piperazin-1-yl]ethanol |
| Loxapine | 97 | 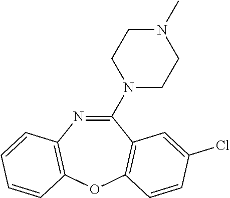 [SMILES: CN1CCN(C2=Nc3ccccc3Oc3ccc(Cl)cc32)CC1] | 2-Chloro-11-(4-methylpiperazin- 1-yl)dibenzo[b,f][1,4]oxazepine |
| Octoclothepin | 97 | 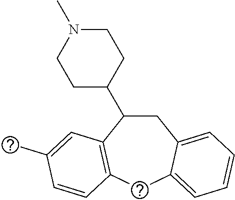 [SMILES: Cc1ccc2c(c1)C(C1CCN(C)CC1)Cc1ccccc1C2] | 1-(8-Chloro-10,11- dihydrodibenzo[b,f\|thiepin-10- yl)-4-methyl-piperazine |
| Perphenazine | 95 | 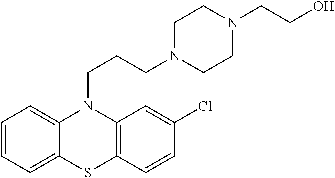 [SMILES: OCCN1CCN(CCCN2c3ccccc3Sc3ccc(Cl)cc32)CC1] | 2-[4-[3-(2-chloro-10H- phenothiazin-10-yl) propyl]piperazin-1-yl]ethanol |
| Prochlorperazine | 83 | 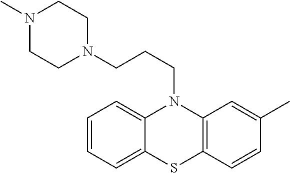 | 2-chloro-10-[3-(4-methyl-1- piperazinyl)propyl]- 10H-phenothiazine |
| Promazine | 88 | 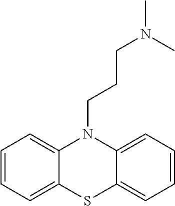 [SMILES: CN(C)CCCN1c2ccccc2Sc2ccccc21] | N,N-dimethyl-3-(10H- phenothiazin-10-yl)-propan-1- amine |
| Propionyl- promazine | 85 | 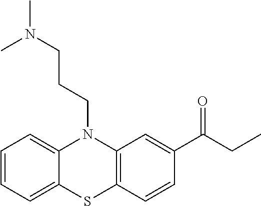 [SMILES: CCC(=O)c1ccc2c(c1)N(CCCN(C)C)c1ccccc1S2] | 1-[10-[3- (dimethylamino)propyl] phenothiazin-2-yl]propan-1-one |
| Risperidone | 83 | 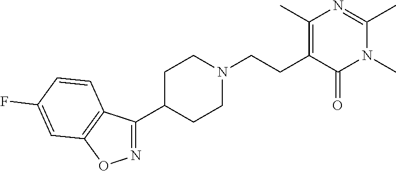 [SMILES: Cc1nc(C)n(C)c(=O)c1CCN1CCC(c2noc3cc(F)ccc23)CC1] | 4-[2-[4-(6- fluorobenzo[d]isoxazol-3-yl)- 1-piperidyl]ethyl]-3-methyl- 2,6-diazabicyclo[4.4.0]deca-1,3- dien-5-one |
| Triflupromazine | 88 | 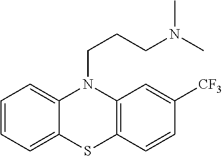 [SMILES: CN(C)CCCN1c2ccccc2Sc2ccc(C(F)(F)F)cc21] | N,N-dimethyl-3-[2- (trifluoromethyl)-10H- phenothiazin-10-yl]propan-1- amine |
| Trifluoperazine | 81 | 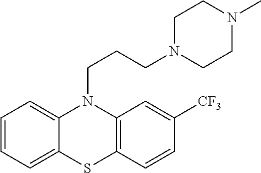 [SMILES: CN1CCN(CCCN2c3ccccc3Sc3ccc(C(F)(F)F)cc32)CC1] | 10-[3-(4-methylpiperazin-1- yl)propyl]- 2-(trifluoromethyl)-10H- phenothiazine |
| Thiothixene | 86 | 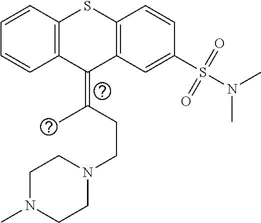 [SMILES: C/C(CCN1CCN(C)CC1)=C1\c2ccccc2Sc2ccc(S(=O)(=O)N(C)C)cc21] | (9Z)-N,N-dimethyl-9-[3-(4- methylpiperazin-1- yl)propylidene]-9H- thioxanthene-2-sulfonamide |
| Thioridazine | 86 | 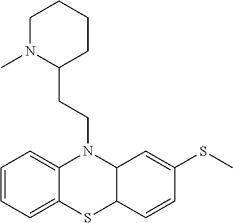 [SMILES: CSC1=CC2C(C=C1)Sc1ccccc1N2CCC1CCCCN1C] | 10-{2-[(RS)-1-Methylpiperidin- 2-yl]ethyl}- 2-methylsulfanylphenothiazine |
| Amperozide | 83 | 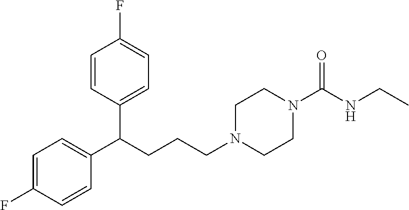 [SMILES: CCNC(=O)N1CCN(CCCC(c2ccc(F)cc2)c2ccc(F)cc2)CC1] | 4-[4,4-bis(4-fluorophenyl)butyl]- N-ethylpiperazine-1-carboxamide |
| Pirenperone | 90 | 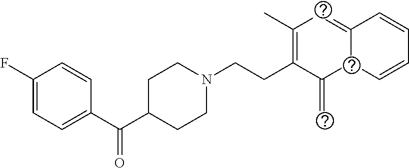 [SMILES: C=C1C(CCN2CCC(C(=O)c3ccc(F)cc3)CC2)=C(C)C=C2C=CC=CC12] | 3-[2-[4-(p- fluorobenzoyl)piperidino]ethyl]- 2-methyl-4h-pyrido[1,2- a]pyrimidin-4-one |
| Ritanserin | 83 | 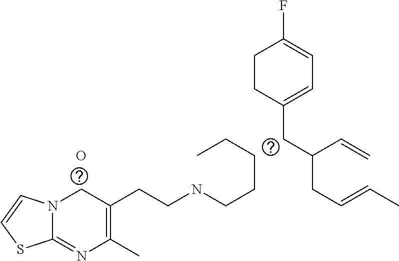 | 6-[2-[4-[bis(4- fluorophenyl)methylidene] piperidin-1-yl]ethyl]-7-methyl- [1,3]thiazolo[2,3-b]pyrimidin-5- one |
| Ketotifen | 96 | 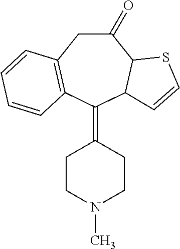 [SMILES: CN1CCC(=C2c3ccccc3CC(=O)C3SC=CC23)CC1] | 4-(1-methylpiperidin-4-ylidene)- 4,9-dihydro-10H- benzo[4,5]cyclohepta[1,2- b]thiophen-10-one |
| Promethazine | 95 | 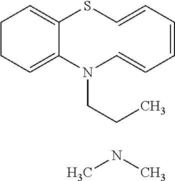 | (RS)-N,N-dimethyl-1-(10H- phenothiazin-10-yl)propan-2- amine |
| Benztropine | 89 | 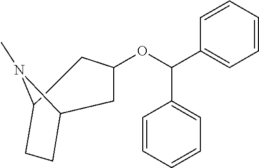 [SMILES: CN1C2CCC1CC(OC(c1ccccc1)c1ccccc1)C2] | (3-endo)-3-(diphenylmethoxy)-8- methyl-8-azabicyclo[3.2.1]octane |
| Amoxapine | 90 | 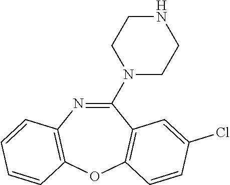 [SMILES: Clc1ccc2c(c1)C(N1CCNCC1)=Nc1ccccc1O2] | 2-Chloro-11-(piperazin-1- yl)dibenzo[b,f][1,4]oxazepine |
| Imipramine | 96 | 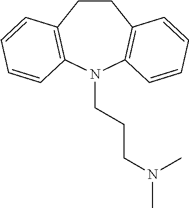 [SMILES: CN(C)CCCN1c2ccccc2CCc2ccccc21] | 3-(10,11-dihydro-5H- dibenzo[b,f]azepin-5-yl)-N,N- dimethylpropan-1-amine |
| Maprotiline | 82 | 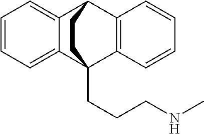 | N-Methyl-9,10- ethanoanthracene-9(10H)- propanamine |
| Nortriptyline | 96 | 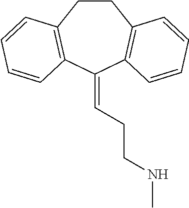 [SMILES: CNCCC=C1c2ccccc2CCc2ccccc21] | 3-(10,11-dihydro-5H- dibenzo[a,d]cyclohepten-5- ylidene)-N-methyl-1- propanamine |
| Protriptyline | 82 | 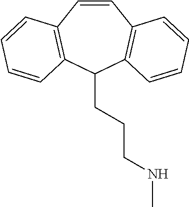 [SMILES: CNCCCC1c2ccccc2C=Cc2ccccc21] | 3-(5H-dibenzo[a,d][7]annulen-5- yl)-N-methylpropan-1-amine |
| Trimipramine | 87 | 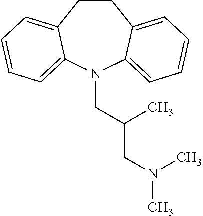 [SMILES: CC(CN(C)C)CN1c2ccccc2CCc2ccccc21] | (±)-3-(10,11-dihydro-5H- dibenzo[b,f]azepin-5-yl)-N,N,2- trimethylpropan-1- |

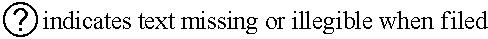

Table 5
primer pairs and experimental conditions used
in RT-PCR analysis of Aadop1 and Aadop2 transcripts.

| | | | | | Presence |
|---|---|---|---|---|---|
| | | Exons | | | (+)/ |
| | | joined | Expected | Annealing | Absence |
| | | by | Product | Tempera- | (-) of |
| | Primer name/ | RT-PCR | Size | ture | RT-PCR |
| Gene | Sequence | Product | (bp) | (° C.) | Product^{a} |
| Aadop1 | Aadop1_Full_F | 1-4 | 1233 | 45/50 | -/- |
| | 5′-AATACGATTGGGATTTTTTG-3′ | | | | |
| | Aadop1_Full_R | | | | |
| | 5′-GATGGCGGATACCTGTTCGAG-3′ | | | | |
| | Aadop1_Full_1F | 1-2 | 224 | 50 | + |
| | 5′-TTTCTCTCCGTAGCCGGTAA-3′ | | | | |
| | Aadop1_Full_1R | | | | |
| | 5′-GCGGTTGAACACATGACATC-3′ | | | | |
| | Aadop1_Full_1F | 1-4 | 1058 | 50 | + |
| | 5′-TTTCTCTCCGTAGCCGGTAA-3′ | | | | |
| | Aadop1_Full_2R | | | | |
| | 5′-GGCGGATACCTGTTCGAGAT-3′ | | | | |
| Aadop2 | Aadop2_Full_F | 1-4 | 1425 | 50 | + |
| | 5′-AATAATCGAACTGACTTCTAC-3′ | | | | |
| | Aadop2_Full_R | | | | |
| | 5′-GATATACGTCTGCTCGCAAGAG-3′ | | | | |

^{a}Data reflect RT-PCR results using total RNA samples extracted from adult female A. aegypti.

TABLE 6
Comparison of transmembrane (TM) domains of A. aegypti
AaDOP1 and AaDOP2 and related D₁-like dopamine receptors.

| | Percent amino acid identify in TM domains^{a} | | | | | | | |
|---|---|---|---|---|---|---|---|---|
| | D. melanogaster | | A. mellifera | | | | | |
| Recep- | D- | Dop | Am | Am | I. scapularis | | H. sapiens | |
| tor | Dop1 | R99B | DOP1 | DOP2 | Isdop1 | Isdop2 | D1 | D2 |
| Aa | 88 | 47 | 81 | 44 | 70 | 47 | 54 | 52 |
| DOP1 | | | | | | | | |
| Aa | 48 | 97 | 51 | 89 | 48 | 73 | 51 | 47 |
| DOP2 | | | | | | | | |

^{a}Calculations of percent identify were based on the aligned TM sequences shown in FIG. 8.

TABLE 7
Summary of selected amino acid features of Aedes aegypti AaDOP1 and AaDop2.

| | Amino acids in | Amino acids in |
|---|---|---|
| Protein features | AaDOP1 | AaDOP2 |
| Total size^{a} | 412 | 476 |
| Size of N-terminus^{a} | 41 | 57 |
| Size of intracellular loops I, II, III^{a} | 10, 20, 62 | 10, 20, 115 |
| Size of extracellular loops I, II, III^{a} | 14, 28, 7 | 15, 18, 9 |
| Size of carboxyl tail^{a} | 61 | 63 |
| 1-4 N linked glycosylation sites (N- | N.P.^{g} | N3, N19, N24, N46 |
| terminus) | | |
| Conserved cysteines in extracellular | C115, C204 | C132, C211 |
| loops 1-II^{b} | | |
| C-terminus palmitoylation sites | C370, C371 | C426, C428 |
| Protein kinase A/C phosphorylation | S72, T155, S245, | S166, T172, T250, |
| (Intracellular loops and C-terminus) | S262, S269, S72, | S252, T278, T305, |
| | T155, S245, S262, | S339, S341, T440, |
| | S269, | S446, S451, S456, |
| | | T457, S459, S470 |
| Conserved aspartate in TM II + III^{c} | D88, D122 | D104, D139 |
| Conserved “DRY” motif^{d} | D139, R140, Y141 | D156, R157, Y158 |
| Conserved serines in TM V^{e} | S216, S217, S220 | S223, S224, S227 |
| Conserved aromatic residues in TM V^{f} | F221 | F228 |
| Conserved aromatic residues in TM VI^{f} | W308, F311, F312 | W368, F371, F372 |

^{a}Values refer to the number of amino acids composing these features
^{b}Presumed to form a disulfide bond for protein stabilization
^{c}Predicted as important for binding the amine moieties of catecholamines
^{d}Implicated in G-protein coupling
^{e}Predicted to form hydrogen bonds with catechol hydroxyl groups
^{f}Aromatic residues implicated in ligand interaction
^{g}N.P. = not predicted

TABLE 8
In vivo Aedes aegypti L3 larvae time course experiment
showing percent mortality of the mosquito population over a 72
hour period following exposure to compounds from FIG. 12.

| Compound ^{a} | 0.5 hr | 1 hr | 1.5 hr | 2 hr | 2.5 hr | 3 hr |
|---|---|---|---|---|---|---|
| Diphenhydramine HCL | 0 | 10 | 0 | 10 | 0 | 0 |
| LY310762 HCL | 0 | 0 | 0 | 0 | 0 | 0 |
| Ketanserin Tartrate | 0 | 0 | 0 | 0 | 0 | 0 |
| Methiothepin Maleate | 10 | 0 | 50 | 20 | 20 | 0 |
| Cis-flupenthixol diHCL | 10 | 30 | 30 | 10 | 10 | 10 |
| Pirenzepine diHCL | 0 | 0 | 0 | 0 | 0 | 0 |
| Asenapine Maleate | 70 | 20 | 0 | 10 | 0 | 0 |
| Chlorprothiexene HCl | 30 | 30 | 20 | 10 | 0 | 0 |
| Amperozide HCl | 90 | 0 | 10 | 0 | 0 | 0 |
| Mianserin HCl | 0 | 20 | 10 | 10 | 10 | 20 |
| Loxapine Succinate Salt | 30 | 0 | 40 | 30 | 0 | 0 |
| Chlorpromazine HCl | 70 | 0 | 10 | 10 | 0 | 0 |
| Benztropine Mesylate | 0 | 0 | 0 | 20 | 20 | 0 |
| Negative Control | 0 | 0 | 0 | 0 | 0 | 2 |
| (water only)^{b} | | | | | | |
| | | | | | | Cumulative |
| | | | | | | % Mortality |
| Compound | 3.5 hr | 4 hr | 24 hr | 48 hr | 72 hr | (72 hrs) |
| Diphenhydramine HCL | 0 | 0 | 50 | 10 | 0 | 80 |
| LY310762 HCL | 0 | 0 | 0 | 10 | 0 | 10 |
| Ketanserin Tartrate | 0 | 0 | 0 | 0 | 0 | 0 |
| Methiothepin Maleate | 0 | 0 | 0 | 0 | 0 | 100 |
| Cis-flupenthixol diHCL | 0 | 0 | 0 | 0 | 0 | 100 |
| Pirenzepine diHCL | 0 | 0 | 0 | 0 | 0 | 0 |
| Asenapine Maleate | 0 | 0 | 0 | 0 | 0 | 100 |
| Chlorprothiexene HCl | 0 | 0 | 0 | 0 | 0 | 90 |
| Amperozide HCl | 0 | 0 | 0 | 0 | 0 | 100 |
| Mianserin HCl | 10 | 0 | 10 | 0 | 0 | 90 |
| Loxapine Succinate Salt | 0 | 0 | 0 | 0 | 0 | 100 |
| Chlorpromazine HCl | 0 | 0 | 10 | 0 | 0 | 100 |
| Benztropine Mesylate | 20 | 0 | 40 | 0 | 0 | 100 |
| Negative Control | 0 | 0 | 0 | 0 | 0 | 2 |
| (water only)^{b} | | | | | | |

^{a} Chemistries were tested in duplicate at a single dose-point of 400 uM.
^{b}Represents average of seven technical replicates.

TABLE 9
Potency of antagonist chemistries against
the AaDOP2 target using HTRF cAMP assay

| Compound | IC₅₀ ± S.E.M. (nM) |
|---|---|
| Methiothepin | 0.25 ± 0.05 |
| Asenapine | 0.30 ± 0.06 |
| Cis-(Z)-flupenthixol | 0.35 ± 0.07 |
| Chlorprothixene | 1.2 ± 0.39 |
| Loxapine | 5.9 ± 1.4 |
| Amitriptyline | 6.5 ± 0.92 |
| Cyproheptadine | 6.5 ± 1.9 |
| Olanzapine | 11 ± 2.2 |
| Clozapine | 14 ± 2.9 |
| Chlorpromazine | 17 ± 0.88 |
| Doxepin | 20 ± 6.2 |
| Amoxapine | 20 ± 8.4 |
| R59-022 | 53 ± 13 |
| Clomipramine | 56 ± 18 |
| Mianserin | 130 ± 24 |
| Nortriptyline | 140 ± 50 |
| Risperidone | 150 ± 41 |
| (+)-Butaclamol | 190 ± 27 |
| Benztropine | 340 ± 41 |
| Imipramine | 360 ± 30 |
| Ritanserin | 500 ± 110 |
| Amperozide | 570 ± 110 |
| Protriptyline | 600 ± 250 |
| Norclomipramine | 670 ± 35 |
| Pirenperone | 680 ± 98 |
| Ketotifen | 750 ± 180 |
| SCH23390 | 1,300 ± 340 |
| LY310762 | 3,000 ± 820 |
| Ketanserin | 3,200 ± 360 |
| Desipramine | 3,300 ± 600 |
| Haloperidol | 4,300 ± 1000 |
| Aripiprazole | 6,500 ± 770 |
| Diphenhydramine | 7,500 ± 2800 |
| Loratidine | 18,000 ± 1800 |
| Trazodone | No Inhibition^{a} |
| Atomoxetine | No Inhibition^{a} |
| Fluoxetine | No Inhibition^{a} |
| Fluvoxamine | No Inhibition^{a} |
| Venlafaxine | No Inhibition^{a} |
| Buspirone | No Inhibition^{a} |
| Amitraz | No Inhibition^{a} |

^{a}<10% inhibition at 3 μM test compound.

TABLE 10
Toxicity of AaDOP2 antagonists to Aedes aegypti larvae
in a high-throughput in vivo screen at 24, 48 and 72 hours.

| Compound^{a} | 24 hours | 48 hours | 72 hours |
|---|---|---|---|
| Asenapine | 100 | 100 | 100 |
| Chlorpromazine | 100 | 100 | 100 |
| Benztropine | 100 | 100 | 100 |
| Methiothepin | 100 | 100 | 100 |
| Cis-flupenthixol | 100 | 100 | 100 |
| Loxapine | 97 | 100 | 100 |
| Chlorprothixene | 87 | 93 | 100 |
| Mianserin | 97 | 97 | 97 |
| Amperozide | 93 | 93 | 93 |
| Clomipramine | 70 | 93 | 93 |
| Amitriptyline | 63 | 87 | 93 |
| Diphenhydramine | 63 | 77 | 83 |
| Imipramine | 53 | 63 | 80 |
| Nortriptyline | 43 | 63 | 73 |
| Norclomipramine | 40 | 63 | 70 |
| Fluoxetine | 43 | 53 | 53 |
| Protriptyline | 37 | 43 | 53 |
| Desipramine | 30 | 40 | 43 |
| Fluvoxamine | 27 | 33 | 43 |
| Tomoxetine | 20 | 30 | 30 |
| SCH23390 | 3 | 23 | 47 |
| Venlafaxine | 3 | 7 | 13 |
| LY310762 HCL | 0 | 3 | 3 |
| Ketanserin | 0 | 0 | 0 |
| Pirenzepine | 0 | 0 | 0 |
| Control^{b} | 0 | 1 | 3 |

^{a}Results represent three independent experiments;
^{b}Control (water only).

TABLE 11
Toxicity of AaDOP2 antagonists to Aedes aegypti
larvae in concentration assays.

| Compound | LC₅₀ |
|---|---|
| Asenapine (n = 3) | 40 μM |
| Cis-(z)-flupenthixol (n = 4) | 88 μM |
| Chlorpromazine (n = 7) | 92 μM |
| Amperozide (n = 2) | N.D. |
| Methiothepin^{†} | — |
| Amitriptyline | 78 μM |

N.D. Not determined
^{†}Experiment ongoing
LC 50, concentration that kills 50% of Ae. aegytpi population.

TABLE 12
Toxicity of AaDOP2 antagonists to adult male
Blatella germanica in cockroach contact assay.

| | % Mortality | | | | |
|---|---|---|---|---|---|
| | 24 | 48 | 72 | 120 | 240 |
| Compound^{a} | hrs | hrs | hrs | hrs | hrs |
| 200 mM Amitriptyline^{b} | 5 | 20 | 25 | 30 | 35 |
| 200 mM Chlorpromazine^{c} | 3 | 10 | 10 | 14 | 17 |
| 200 mM Cis-(z)-flupenthixol | 10 | 10 | 10 | 20 | 20 |
| 200 mM Asenapine^{b} | 5 | 5 | 10 | 10 | 10 |
| 200 mM Amperozide | 0 | 0 | 0 | 5 | 5 |
| Negative control^{d} | 0 | 0 | 0 | 10 | 10 |
| Positive control (Indoxacarb) | 50 | 50 | 70 | 100 | 100 |

^{a}Compounds listed in rank order of toxicity;
^{b}Results represent two independent experiments;
^{c}Represents three independent experiments;
^{d}Negative control (DMSO:EtOH 1:1 v:v).

TABLE 13
Toxicity of AaDOP2 antagonists to adult Reticulitermes flavipes
in termite immersion assay.

| | % Mortality | | | | |
|---|---|---|---|---|---|
| | 24 | 48 | 72 | 120 | 240 |
| Compound^{a} | hrs | hrs | hrs | hrs | hrs |
| 100 mM Asenapine | 0 | 0 | 20 | 50 | 100 |
| 100 mM Cis-(z)-flupenthixol | 50 | 50 | 50 | 70 | 80 |
| 100 mM Amperozide | 50 | 50 | 50 | 60 | 70 |
| 100 mM Amitriptyline | 40 | 40 | 60 | 70 | 70 |
| 100 mM Chlorpromazine | 50 | 50 | 50 | 60 | 60 |
| Negative control^{b} | 10 | 10 | 10 | 10 | 20 |
| Positive control (Fipronil^{c}) | 90 | 100 | 100 | 100 | 100 |

^{a}Compounds listed in rank order of toxicity;
^{b}Negative control (DMSO:EtOH 1:1 v:v).
^{c}Fipronil (500 μM).

TABLE 14
Additional compounds confirmed in using the in vitro Concentration Response Curve assay as antagonists of the AaDOP2
receptor. Provided is the name and IUPAC designation of each compound, together with the corresponding Inhibitory Concentration
(IC50) value in μM determined in the assay (n = 2) relative to the control antagonist, Amitriptyline. Compounds
with very low IC50 values are particularly potent in the in vitro cell assay and are of particular interest.

| | | Avg |
|---|---|---|
| Compound Name | | IC50 |
| (or Vendor ID) | IUPAC/Chemical Name | (μM) |
| Amitriptyline | 3-(10,11-dihydro-5H-dibenzo[a,d]cycloheptene-5-ylidene)-N,N- | 0.007 |
| (control) | dimethylpropan-1-amine | |
| Maprotiline HCl | 9-(γ-Methylaminopropyl)-9,10-dihydro-9,10-ethanoanthracene | 1.370 |
| | hydrochloride | |
| Tioridazine HCl | 10-[2-(1-Methyl-2-piperidyl)ethyl]-2-(methylthio)-10H-phenothiazine | 0.003 |
| | hydrochloride | |
| Prochlorperazine | 2-Chloro-10-[3[(4-methyl-1-piperazinyl)propyl]-10H-phenothiazine | 0.370 |
| dimaleate salt | dimaleate | |
| Miconazole nitrate | 1-(2,4-Dichloro-β-[(2,4-dichlorobenzyl)oxy]phenethyl)imidazole | 7.353 |
| Toremifene citrate | (Z)-2-(4-(4-Chloro-1,2-diphenyl-1-butenyl)phenoxy)-N,N- | 44.900 |
| salt | dimethylethanamine | |
| Carvedilol | 1-(9H-Carbazol-4-yloxy)-3-[[2-(2-methoxyphenoxy)ethyl]amino]-2- | 8.498 |
| | propanol | |
| Phentolamine HCl | 2-[N-(3-Hydroxyphenyl)-p-toluidinomethyl]-2-imidazolidine | 9.027 |
| | hydrochloride | |
| Pergolide Mesylate | 8β-[(Methylthio)methyl]-6-propylergoline methanesulfonate salt | 0.704 |
| Azelastine HCl | 4-[(4-chlorophenyl)methyl]-2-(1-methylazepan-4-yl)phthalazin-1-one | 47.450 |
| | hydrochloride | |
| Peropirone Hcl | (3aR,7aS)-2-{4-[4-(1,2-benzisothiazol-3-yl)piperazin-1- | 0.186 |
| | yl]butyl}hexahydro-1H-isoindole-1,3(2H)-dione | |
| Nefazodone HCl | 2-[3-[4-(3-chlorophenyl)-1-piperazinyl]propyl]-5-ethyl- | 4.878 |
| | 2,4-dihydro-4-(2-phenoxyethyl)-3H-1,2,4-triazol-3-one | |
| | hydrochloride | |
| Benproperine | (RS)-1-[2-(2-benzylphenoxy)-1-methylethyl]piperidine | 4.513 |
| phosphate | | |
| Oxiconazole Nitrate | (E)-[1-(2,4-dichlorophenyl)-2-(1H- | 10.975 |
| | imidazol-1-yl)ethylidene][(2,4-dichlorophenyl)methoxy]amine | |
| Flecainide Acetate | N-(2-Piperidylmethyl)-2,5-bis-(2,2,2-trifluoroethoxy)benzamide | 0.318 |
| | acetate salt | |
| Desloratidine | 8-Chloro-6,11-dihydro-11-(4-piperidinylidene)-5H- | 1.171 |
| | benzo[5,6]cyclohepta[1,2,b]pyridine | |
| Perphenazine | 2-[4-[3-(2-chloro-10H-phenothiazin-10-yl) | 0.101 |
| | propyl]piperazin-1-yl]ethanol | |
| Fluphenazine diHCl | 4-[3-[2-(Trifluoromethyl)-10H-phenothiazin-10-yl]propyl]-1- | 0.015 |
| | piperazineethanol dihydrochloride | |
| (TimTec) | | |
| ST002262 | ethyl 6-bromo-5-methoxy-2-methyl-1-phenylindole-3-carboxylate | 4.855 |
| ST010134 | 6-(3-hydroxy-3-methyl-but-1-ynyl)-benzo(de)isochromene-1,3-dione | 0.952 |
| ST024096 | 4-{[2-(4-chlorophenyl)quinazolin-4-yl]amino}benzoic acid, chloride | 0.107 |
| ST027415 | (2-methylphenyl){2-[(2-methylphenyl)amino]quinazolin-4-yl}amine | 8.860 |
| ST029812 | methyl 4-{[2-(ethylpropyl)quinazolin-4-yl]amino}benzoate | 8.930 |
| ST029892 | (3-chloro-4-methylphenyl)[2-(ethylpropyl)quinazolin-4-yl]amine | 4.506 |
| ST038696 | 5-(3-nitro-2H-chromen-2-yl)-2H-benzo[d]1,3-dioxolane | 5.505 |
| ST070482 | 5-methyl-2-(methylethyl)cyclohexyl 2-oxo-2-phenylacetate | 32.400 |
| ST052085 | 5-[(6-bromo(2H-benzo[d]1,3-dioxolen-5-yl))methylene]-2-imino-3- | 74.184 |
| | [4-(trifluorome thoxy)phenyl]-1,3-thiazolidin-4-one | |
| ST071653 | ethyl | 1.648 |
| | 2-amino-4-(2-chloro-5-nitrophenyl)-6-(hydroxymethyl)-8-oxo-4H- | |
| | pyrano[3,2-b]pyran-3-carboxylate | |
| ST075014 | N-(4-(2H,3H-benzo[3,4-e]1,4-dioxin-6-yl)(1,3-thiazol-2-yl))(3,4- | 8.309 |
| | dimethoxyphenyl)carboxamide | |
| ST077642 | N-[2-(5-methoxyindol-3-yl)ethyl]-2-(1-methyl-2-phenylindol-3-yl)-2- | 17.635 |
| | oxoacetamide | |
| ST088437 | 2-methyl-5-benzyl-1,2,3,4-tetrahydropyridino[4,3-b]indole | 1.970 |
| ST092311 | 5-acetyl-2H-benzo[d]1,3-dioxolene | 5.820 |
| ST095663 | (phenylethyl)(2-piperazinylquinazolin-4-yl)amine | 4.841 |
| (ChemDiv, Inc) | | |
| G869-0096 | N-(4-((4-methyl-6-(phenylamino)pyrimidin-2-yl)amino)phenyl)propionamide | 4.741 |
| G869-0097 | N-(4-((4-methyl-6-(phenylamino)pyrimidin-2-yl)amino)phenyl)butyramide | 6.052 |
| G869-0098 | N-(4-((4-methyl-6-(phenylamino)pyrimidin-2-yl)amino)phenyl)isobutyramide | 3.101 |
| G869-0099 | N-(4-((4-methyl-6-(phenylamino)pyrimidin-2-yl)amino)phenyl)pentanamide | 5.947 |
| G869-0100 | N-(4-((4-methyl-6-(phenylamino)pyrimidin-2-yl)amino)phenyl)pivalamide | 2.077 |
| G869-0101 | 3-methyl-N-(4-((4-methyl-6-(phenylamino)pyrimidin-2-yl)amino)phenyl)butanamide | 5.500 |
| G869-0105 | N-(4-((4-methyl-6-(phenylamino)pyrimidin-2-yl)amino)phenyl)cyclohexanecarboxamide | 11.556 |
| G869-0121 | 3,5-dimethoxy-N-(4-((4-methyl-6-(phenylamino)pyrimidin-2-yl)amino)phenyl)benzamide | 2.216 |
| G869-0127 | 2-chloro-N-(4-((4-methyl-6-(phenylamino)pyrimidin-2-yl)amino)phenyl)benzamide | 7.510 |
| G869-0268 | N-(4-((4-((4-methoxyphenyl)amino)-6-methylpyrimidin-2-yl)amino) | 13.218 |
| | phenyl)acetamide | |
| G869-0273 | N-(4-((4-((4-methoxyphenyl)amino)-6-methylpyrimidin-2-yl)amino) | 5.085 |
| | phenyl)pivalamide | |
| G869-0275 | N-(4-((4-((4-methoxyphenyl)amino)-6-methylpyrimidin-2-yl)amino) | 9.021 |
| | phenyl)-3,3-dimethylbutanamide | |
| G869-0304 | 2-chloro-6-fluoro-N-(4-((4-((4-methoxyphenyl)amino)-6-methylpyrimidin-2-yl)amino)phenyl)benzamide | 7.167 |
| G869-0472 | 2-bromo-N-(4-((4-methyl-6-(pyrrolidin-1-yl)pyrimidin-2-yl)amino)phenyl)benzamide | 19.405 |
| G869-0499 | N-(4-((4-methyl-6-(pyrrolidin-1-yl)pyrimidin-2-yl)amino)phenyl)-2- | 9.233 |
| | phenylacetamide | |
| G932-0029 | 6-(4-methylpiperazin-1-yl)-1-phenyl-N-(p-tolyl)-1H-pyrazolo[3,4-d]pyrimidin-4-amine | 4.552 |
| G932-0129 | 6-(4-methylpiperazin-1-yl)-1-phenyl-N-(o-tolyl)-1H-pyrazolo[3,4-d]pyrimidin-4-amine | 3.488 |
| G932-0232 | 2-(4-(1-phenyl-4-(phenylamino)-1H-pyrazolo[3,4-d]pyrimidin-6-yl)piperazin-1-yl)ethanol | 1.174 |
| G932-0245 | 6-(4-ethylpiperazin-1-yl)-N,1-diphenyl-1H-pyrazolo[3,4-d]pyrimidin-4-amine | 0.557 |
| G932-0329 | N-(4-fluorophenyl)-6-(4-methylpiperazin-1-yl)-1-phenyl-1H-pyrazolo[3,4-d]pyrimidin-4-amine | 2.495 |
| G932-0345 | 6-(4-ethylpiperazin-1-yl)-N-(4-fluorophenyl)-1-phenyl-1H-pyrazolo[3,4-d]pyrimidin-4-amin | 4.544 |
| G932-0372 | N4-(4-chlorophenyl)-N6-(2-(diethylamino)ethyl)-1-phenyl-1H-pyrazolo[3,4-d]pyrimidine-4,6-diamine | 5.130 |
| G932-0379 | N-(4-chlorophenyl)-6-(4-methylpiperazin-1-yl)-1-phenyl-1H-pyrazolo[3,4-d]pyrimidin-4-amine | 3.981 |
| G932-0529 | N-(4-methoxyphenyl)-6-(4-methylpiperazin-1-yl)-1-phenyl-1H-pyrazolo[3,4-d]pyrimidin-4-amine | 6.869 |
| G932-0671 | N-(3-fluorophenyl)-6-(4-methylpiperazin-1-yl)-1-phenyl-1H-pyrazolo[3,4-d]pyrimidin-4-amine | 0.585 |
| G932-0674 | 2-(4-(4-((3-fluorophenyl)amino)-1-phenyl-1H-pyrazolo[3,4-d]pyrimidin-6-yl)piperazin-1-yl)ethanol | 1.481 |
| G932-0687 | 6-(4-ethylpiperazin-1-yl)-N-(3-fluorophenyl)-1-phenyl-1H-pyrazolo[3,4-d]pyrimidin-4-amine | 0.769 |
| G932-0818 | N-(benzo[d][1,3]dioxol-5-yl)-6-(4-methylpiperazin-1-yl)-1-phenyl-1H-pyrazolo[3,4-d]pyrimidin-4-amine | 1.654 |

TABLE 15
Lethal Time (LT₅₀) of Compounds in Aedes aegypti
L3 Larval Time Course Assay

| Compound | LT₅₀ (mins) | No. Replicates (n) |
|---|---|---|
| 400 μM Asenapine | 30 | 3 |
| 400 μM Amperozide | 42 | 3 |
| 400 μM Cis-flupenthixol | 54 | 5 |
| 400 μM Chlorpromazine | 178 | 7 |

TABLE 16
Toxicity of AaDOP2 antagonists to adult male
Blatella germanica in cockroach contact assay

| | % Mortality | | | |
|---|---|---|---|---|
| Compound | 24 hrs | 48 hrs | 72 hrs | 120 hrs |
| 200 mM Amitriptyline | 3 | 20 | 23 | 30 |
| 200 mM Asenapine^{a} | 17 | 20 | 27 | 27 |
| 200 mM Chlorpromazine | 7 | 17 | 17 | 20 |
| 200 mM cis-flupenthixol | 3 | 10 | 10 | 20 |
| 200 mMAmperozide^{a} | 0 | 0 | 3 | 7 |
| Negative control | 0 | 0 | 3 | 7 |
| (2 μL DMSO/ethanol) | | | | |
| Positive control (Indoxacarb) | 63 | 63 | 80 | 100 |

Negative control (DMSO:EtOH 1:1 v:v
Results represent three independent experiments (n = 3) unless otherwise specified
^{a}Results represent two independent experiments (n = 2)

Table 17 Summary of amino acid similarity between the conserved TM domain regions of Isdop1 and Isdop2 in Ixodes scapularis with the D₁-like dopamine receptors in Drosophila melanogaster (D-Dop1; Gotzes et al., 1994; Dop R99B (DAMB); Feng et al., 1996; Han et al. 1996), Apis mellifera (DOP1, DOP2: Mustard et al., 2003), and Homo sapiens (D1: Sunahara et al., 1990; D5; Sunahara et al., 1991).

| | Percent amino acid similarity in TM domains^{a} | | | | | |
|---|---|---|---|---|---|---|
| I. scapularis | D. melanogaster | | A. mellifera | | H. sapiens | |
| protein | D-Dop1 | DopR99B | DOP1 | DOP2 | D1 | D5 |
| Isdop1 | 72 | 48 | 65 | 45 | 53 | 52 |
| Isdop2 | 47 | 74 | 49 | 74 | 47 | 47 |

^{a}Calculation based on the number of conserved amino acids among the combined TM domain sequences aligned in FIG. 15.

Table 18 Summary of key features in Isdop1 and Isdop2 based on their deducted amino acid sequences. The number of amino acids composing the N- and C-termini and the intracellular and extracellular loops were determined relative to the predicted transmembrane (TM) domain sequences shown in FIG. 15

| | Amino acids in | Amino acids in |
|---|---|---|
| Protein features | Isdop1 | Isdop2 |
| 1-4N-linked glycosylation sites | N24, N25 | N8, N26, N37, |
| (N-terminus) | | N50, N61 |
| Conserved cysteines in | C121, C205 | C150, C229 |
| extracellular loops I, II | | |
| C-terminus palmitoylation sites | C354, C355 | C420, C421, C422, |
| | | C423 |
| Protein kinase A/C | T161, T251, T262, | T109, T190, S275, |
| phosphorylation (Intracellular | S366, S369, T372, | T290, T307, S334, |
| loops II, III and C-terminus) | S375, T380, S385, | S446, S451 |
| | S403, S415, S418 | |
| Conserved aspartate In TMII, TMIII | D94, D128 | D122, D157 |
| Conserved “DRY” motif | D145, R146, F147 | D174, R175, Y176 |
| Conserved serines in TMV | S217, S218, S221 | S241, S242, S245 |
| Conserved aromatic residue in TMV | F222 | F246 |
| Conserved aromatic residues in | W294, F297, F298 | W361, F364, F365 |
| TMVI | | |
| Site of N-terminus^{a} | 47 | 75 |
| Size of intracellular loops | 10, 20, 47 | 10, 20, 90 |
| I, II, III^{a} | | |
| Size of extracellular loops | 14, 23, 7 | 15, 18, 10 |
| I, II, III^{a} | | |
| Size of carboxyl tail^{a} | 88 | 50 |

^{a}Values refer to the number of amino acids composing these features.

Table 18 Activity of Isdop1 and Isdop2 following stimulation with biogenic amines. Cyclic AMP accumulation was measured in the absence (basal) or presence of the indicated biogenic amines (10 μM). All experiments were conducted in the presence of 300 nM propranolol to block activation of endogenous β adrenergic receptors in HEK cells. The stimulatory response is shown as fold-over-basal, and the data include the mean±standard error of six independent experiments assayed in duplicate, *p<0.05, **p<0.01, ***p<0.001 using one-way ANOVA with Dunnett's post-hoc test comparing each drug treatment condition to basal.

Stimulatory response (fold-over-basal)

| Biogenic amine | Isdop1 | Isdop2 |
|---|---|---|
| Dopamine | 1.58 ± 0.13** | 10.02 ± 3.51*** |
| Epinephrine | 1.48 ± 0.17* | 1.99 ± 0.47 |
| Histamine | 1.02 ± 0.10 | 1.55 ± 0.28 |
| Norepinephrine | 1.46 ± 0.10* | 2.43 ± 0.65 |
| Octapamine | 1.01 ± 0.07 | 1.20 ± 0.24 |
| Serotonin | 1.06 ± 0.08 | 1.52 ± 0.20 |
| Tyramine | 1.34 ± 0.11 | 1.27 ± 0.22 |

^{a} Calculation based on the number of conserved amino acids among the combined TM domain sequences aligned in FIG. 15.

Table 19 Hit compounds with antagonist properties at Isdop2 identified in the LOPAC¹²⁸⁰ chemical library screen.

| | % In- | |
|---|---|---|
| | hibi- | |
| Hit class & Compound | tion^{a} | Mode of action |
| Dopamine receptor ligands (26) | | |
| R( [symbol] )-SCH-23390 hydrochloride^{b,c} | 77 | D₁ receptor antagonist |
| BP 897^{d} | 71 | Partially selective D₃ |
| | | receptor agonist |
| (±)-Butaclamol hydrochloride | 86 | DA receptor antagonist |
| (+)-Butaclamol hydrochloride^{c} | 93 | DA receptor antagonist |
| Chlorpromazine hydrochloride^{d} | 92 | DA receptor antagonist |
| Chlorprothixene hydrochloride | 97 | D₂ receptor antagonist |
| Clozapine^{c} | 89 | D₄ receptor selective |
| | | antagonist |
| Cortexolone maleate^{d} | 70 | D₂ receptor antagonist |
| Fluphenazine dihydrochlorid^{e} | 79 | DA receptor antagonist |
| Cis-(Z)-Flupenthixol dihydrochloride^{c} | 92 | DA receptor antagonist |
| LE300 | 100 | D₁ receptor antagonist |
| R(+)-Lisuride hydrogen maleate^{d} | 84 | D₂ receptor antagonist & |
| | | 5-HT receptor ligand |
| Loxapine succinate | 93 | N.D. |
| (±)-Octoclothepin maleate | 99 | D₂ & 5-HT receptor |
| | | antagonist |
| Perphenazine | 76 | D₂ receptor antagonist/s |
| | | receptor agonist |
| Prochlorperazine dimaleate | 86 | DA receptor antagonist |
| Promazine hydrochloride | 87 | D₂ receptor antagonist |
| Propionylpromazine hydrochloride | 94 | D₂ receptor antagonist |
| Risperidone | 100 | D₂ & 5-HT receptor |
| | | antagonist |
| SKF 75670 hydrobromide^{d} | 68 | Atypical D₁ receptor |
| | | agonist |
| SKF 83959 hydrobromide^{d} | 87 | Atypical D₁ receptor |
| | | agonist |
| R(+)-Terguride^{d} | 68 | Partial DA receptor |
| | | agonist |
| Triflupromazine hydrochloride | 83 | D₂ receptor antagonist |
| Trifluoperazine dihydrochloride | 76 | DA receptor & |
| | | Calmodulin antagonist |
| Thiothixene hydrochloride | 87 | DA receptor antagonist |
| Thioridazine hydrochloride | 85 | DA receptor & Ca²⁺ |
| | | channel antagonist |
| Serotonin receptor ligands (9) | | |
| Amperozide hydrochloride | 69 | 5-HT receptor antagonist |
| Cyclobenzaprine^{d} | 95 | 5-HT₂ receptor |
| | | antagonist |
| Cyproheptadine^{d} | 93 | 5-HT₂ receptor |
| | | antagonist |
| LY-310,762 hydrochloride | 72 | 5-HT_{1D} selective |
| | | antagonist |
| Mianserin hydrochloride^{c} | 87 | 5-HT receptor antagonist |
| Methiothepin mesylate^{c} | 100 | 5-HT₁ selective |
| | | antagonist |
| NAN-190 hydrobromide^{d} | 69 | 5-HT_{1A} selective |
| | | antagonist |
| Pirenperone | 87 | 5-HT₂ antagonist |
| Ritanserin | 77 | 5-HT₂ selective |
| | | antagonist |
| Histamine receptor ligands (3) | | |
| Clemastine fumarate^{d} | 90 | H₁ receptor antagonist |
| Orphenadrine hydrochloride^{d} | 81 | H₁ & mAChR receptor |
| | | antagonist |
| Promethazine hydrochloride | 74 | H₁ receptor antagonist |
| Muscarinic ACh receptor ligands (2) | | |
| Aminobenztropine^{d} | 71 | mACh receptor ligand |
| Benztropine mesylate | 84 | mACh receptor |
| | | antagonist |
| Metabotropic Glu receptor ligand (1) | | |
| SIB 1757^{d} | 100 | mGlu5 receptor |
| | | antagonist |
| Adrenergic receptor ligand (1) | | |
| SKF86466^{d} | 86 | α2AAR antagonist |
| Biogenic amine uptake inhibitors (9) | | |
| Amitriptyline hydrochloride^{c} | 87 | N.D. |
| Amoxapine | 92 | NE uptake inhibitor |
| Clomipramine hydrochloride^{d} | 84 | 5-HT uptake inhibitor |
| 40-Chloro-3-alpha-(diphenyl-methoxy) | 80 | DA uptake inhibitor |
| tropane hydrochloride | | |
| Doxepin hydrochloride^{c} | 87 | N.D. |
| Imipramine hydrochloride | 70 | 5-HT &NE uptake |
| | | inhibitor |
| Nortriptyline hydrochloride | 79 | NE uptake inhibitor |
| Protriptyline hydrochloride | 75 | NE uptake inhibitor |
| Trimipramine maleate | 86 | 5-HT &NE uptake |
| | | inhibitor |
| Protein kinase modulators (14) | | |
| CGP-74514A hydrochlorided | 98 | Cdk1 inhibitor |
| Diacylglycerol kinase inhibitor I | 69 | Diacylglycerol kinase |
| | | inhibitor |
| Genistein^{d} | 71 | Tyrosine & |
| | | topoisomerase II kinase |
| | | inhibitor |
| GW5074^{d} | 81 | cRaf1 kinase inhibitor |
| H-7 dihydrochloride^{d} | 73 | PKC, PKA inhibitor |
| Indirubin-3′-oxime^{d} | 93 | CDK inhibitor |
| Kenpaullone | 68 | CDK1, CDK2, CDK5 |
| | | inhibitor |
| NSC 95397 | 93 | Cdc25 inhibitor |
| Piceatannol | 100 | Syk & Lck inhibitor |
| Phorbol 12-myristate 13-acetate | 75 | Activates protein kinase |
| | | C |
| Purvalanol A | 84 | CDK inhibitor |
| PD 98,059^{d} | 87 | MAPKK inhibitor |
| Roscovitine^{d} | 91 | CDK inhibitor |
| Rottlerin^{d} | 97 | PKC and CaM III kinase |
| | | inhibitor |
| Ion channel inhibitors (5) | | |
| Dequalinium dichloride^{d} | 75 | K⁺ channel blocker |
| Dihydroouabain^{d} | 76 | Na⁺/K⁺ ATPase inhibitor |
| Ouabain^{d} | 86 | Na⁺/K⁺ ATPase inhibitor |
| Phenamil methanesulfonate^{d} | 72 | Na⁺ channel inhibitor |
| Sanguinarine chloride^{d} | 100 | Mg²⁺ and Na⁺/K⁺ |
| | | ATPase inhibitor |
| Miscellaneous (15) | | |
| Apigenin^{d} | 98 | Arrests cell cycle at |
| | | G2/M phase |
| (S)-(+)-Camptothecin | 97 | DNA topoisomerase I |
| | | inhibitor |
| Cyclosporin A^{d} | 73 | Calcineurin phosphatase |
| | | inhibitor |
| Daidzein^{d} | 77 | Mitochondrial ALDH |
| | | inhibitor |
| 2,3-Dimethoxy-1,4-naphthoquinone^{d} | 68 | Redox cycling reagent |
| Diphenyleneiodonium chloride^{d} | 78 | eNOS inhibitor |
| D-ribofuranosylbenzimidazole^{d} | 82 | Inhibitor of RNA |
| | | synthesis |
| Emetine dihydrochloride hydrate | 100 | Apoptosis inducer; |
| | | protein translation |
| | | inhibitor |
| Idarubicin | 97 | Disrupts topoisomerase |
| | | II |
| Niclosamide | 100 | Uncouples oxidative |
| | | phosphorylation |
| Resveratrol | 100 | Inhibits lipo- & cyclo- |
| | | oxygenase activity |
| Rotenone^{d} | 77 | Inhibits mitochondria |
| | | electron transport |
| Tyrphostin A9^{d} | 87 | PDGF tyrosine kinase |
| | | receptor inhibitor |
| Tyrphostin AG 879^{d} | 75 | TrkA receptor inhibitor |
| Z-L-Phe chloromethyl ketone^{d} | 90 | Chymotrypsin A-gamma |
| | | inhibitor |
| Total number of hits 85 (6.6% hit rate) | | |

Abbreviations: DA, Dopamine; NE, norepinephrine; mGlu, metabotropic glutamate receptor; mACh, muscarinic acetylcholine receptor; CDK, cyclin-dependent kinase; Cdc25, cell division cycle dual-specificity phosphatase; Syk, spleen tyrosine kinase; Lck, lymphocyte-specific tyrosine kinase; MAPKK, mitogen-activated protein kinase kinase; ALDH, aldehyde dehydrogenase; PDGF, platelet-derived growth factor; eNOS, endothelial nitric oxide synthase; CaM, calmodulin; 5-HT, 5-hydroxytryptamine; PKA, protein kinase A; PKC, protein kinase C; TrkA, neurotrophic tyrosine kinase receptor type 1; N.D. not determined.
^{a}Percent inhibition of response to 3 mM dopamine.
^{b}Reference compound used to establish hit criteria.
^{c}Compound selected for in vitro confirmation assays.
^{d}Unique Isdop2 hits (i.e. unique to the current screen, not identified as hits in the AaDOP2 screen, Meyer et al., 2012).

Table 18 Estimated IC₅₀ values (in μM) for select antagonists of the Isdop1 and Isdop2 receptors in confirmation assays using a direct measurement of cAMP. The antagonistic properties are represented by average and 95% confidence intervals of IC₅₀ values based on at least three independent experiments.

| Compound | Isdop1 (10 nM DA) | Isdop2 (30 μM DA) |
|---|---|---|
| Amitriptyline | 77 (20-292) | 2.9 (2.0-4.3) |
| Butaclamol | 3.5 (2.2-.54) | 0.32 (0.13-0.77) |
| Clozapine | 4.7 (2.9-7.5) | 9.3 (6.5-16.2) |
| Cis-(Z)-Flupenthixol | 3.7 (2.5-5.5) | 0.87 (0.62-1.23) |
| Methiothepin | 0.95 (0.63-1.4) | 0.085 (0.04--.17) |
| SCH23390 | 0.057 (0.04-0.079) | 0.12 (0.055-0.27) |

Abbreviation: DA, Dopamine.

TABLE 19
Active compounds identified in AaDOP2 agonist screen against
the LOPAC₁₂₈₀ library. Compound activity in AaDOP2 cells expressed
as a percent of response to 1 μM dihydrexidine (% of DHX).
All compounds were tested at a final concentration 10 μM.

| % of | | |
|---|---|---|
| DHX | Chemical Name | Pharmacological Action |
| 131 | 6,7-ADTN | Dopamine receptor agonist |
| 116 | Dihydrexidine | D1 dopamine receptor agonist |
| 107 | N-Methyldopamine | Dopamine receptor agonist |
| 105 | (±)-Chloro-APB | D1 dopamine receptor agonist. |
| 103 | Dopamine | Endogenous neurotransmitter |
| 101 | SKF 89626 | D1 dopamine receptor agonist |
| 101 | Betamethasone | SAID: glucocorticoid |
| 93 | Agroclavine | Dopamine receptor agonist; ergot alkaloid |
| 90 | (−)-Epinephrine | Endogenous hormone and neurotransmitter |
| 86 | L(−)- | Adrenergic neurotransmitter; |
| | Norepinephrine | vasoconstrictor |
| 81 | (±)-Epinephrine | Adrenoceptor agonist |
| 75 | 1-Aminobenzotriazole | Cytochrome P450 and chloroperoxidase |
| | | inhibitor |
| 73 | EIPA | Selective blocker of Na+/H+ antiport |
| 68 | R(−)-Propyl-NPA | Highly potent D2 dopamine receptor |
| | | agonist |
| 56 | R(−)-N-Allyl-NPA | Dopamine receptor agonist |
| 47 | Methylergonovine | Dopamine antagonist; ergot alkaloid |
| | | Potent and selective D2 dopamine receptor |
| 44 | 3OH-NPA | agonist |
| 38 | N-Oleoyldopamine | Weak CB1 cannabinoid receptor ligand. |
| 38 | Dipropyldopamine | Dopamine receptor agonist |
| 38 | SKF81297 | D1 dopamine receptor agonist |
| 36 | N6-Benzyl-NECA | Selective A3 adenosine receptor agonist |
| 34 | CPCA | Potent A2 adenosine receptor agonist |
| 34 | Forskolin | Activates adenylate cyclase |
| 31 | Fenoldopam | Peripheral D1 dopamine receptor agonist |
| 30 | SKF 83959 | Atypical D1 dopamine receptor agonist |

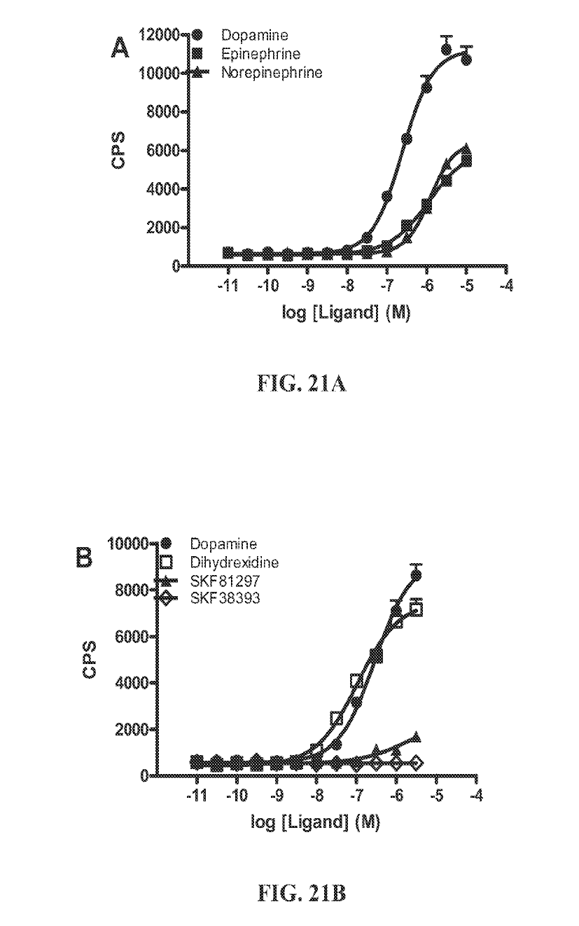

The compounds in Table 19 were identified as candidate agonists of the AaDOP2 target in vitro using the screening methods provided against the LOPAC¹²⁸⁰ library. Follow up CRCs of two biogenic amine “hits” show that both epinephrine and norepinephrine behave as partial agonists at the AaDOP2 receptor (FIG. 21A). Surprisingly, a small set of well characterized mammalian D1 receptor agonists showed weak (i.e. SKF81297=38%) or no activity (i.e. SKF38393<10%) in the screen. The activity of fresh powders of SKF81297 and SKF38393 was compared to two control ligands, dopamine and dihydrexidine (FIG. 21B). The results of these follow up CRC studies are consistent with the observations from the screening exercise and show the weak activity of these synthetic human D1 agonists at the AaDOP2 receptor. These findings support the overall hypothesis that the discovery of unique AaDOP2 agonist chemistries is very feasible. Additionally, the screening data combined with the follow up CRCs support the utility of the AaDOP2 agonist assay for additional drug discovery efforts.

Additional libraries of compounds that can be screened in order to try and identify compounds that have an affinity for gene products that are substantially similar to those encoded by SEQ. ID NOS. 18. These libraries may contain compounds such as: Linear tricyclic compounds, bearing, when possible, hetero atoms in the central ring, including N, O, and S. Compounds with exocyclic bonds, e.g. amitriptyline and doxepin, will be catalytically reduced and the reduced molecules assayed to determine whether that bond is necessary for high activity. One or two members of the series that prove to have high activity will be compared with their secondary and primary amine analogues. For example, nortriptyline is the secondary amine analog of amitriptyline. We might also study the N-methylpiperazine analogues of amitriptyline and doxepin to determine whether an N,N-dimethylamino or N-methylpiperazine is optimum. Still other N,N-dialkyl compounds can also be tested.

Parallel comparisons can also be made of ring-substituted compounds with their non-substituted congeners, e.g., the 8-chloro atom can be removed from clozapine and that molecule (des-chloroclozapine) can be screen to determine if it interacts with these gene products and/or has insecticidal activity.

### REFERENCES

1. Hemingway J, Ranson H (2000) Insecticide resistance in insect vectors of human disease. Annu Rev Entomol 45: 371-391.
2. Hemingway J, Beaty B J, Rowland M, Scott T W, Sharp B L (2006) The Innovative Vector Control Consortium: improved control of mosquito-borne diseases. Trends Parasitol 22: 308-312.
3. Greenwood B M, Fidock D A, Kyle D E, Kappe S H, Alonso P L, et al. (2008) Malaria: progress, perils, and prospects for eradication. J Clin Invest 118: 1266-76.
4. Strader C D, Fong T M, Graziano M P, Tota M R (1995) The family of G-protein-coupled receptors. FASEB J 9: 745-754.
5. Gether U (2000) Uncovering molecular mechanisms involved in activation of G protein-coupled receptors. Endocr Rev 21: 90-113.
6. Filmore D (2004) It's a GPCR world. Modern Drug Disc 7: 24-28.
7. Gamo F-J, Sanz L M, Vidal J, de Cozar C, Alvarez E, et al. (2010) Thousands of chemical starting points for antimalarial lead identification. Nature 465: 305-312.
8. Chen A C, He H, Davey R B (2007) Mutations in a putative octopamine receptor gene in amitraz-resistant cattle ticks. Vet Parasitol 148: 379-383.
9. Hill C A, Fox A N, Pitts R J, Kent L B, Tan P L, et al. (2002) G protein-coupled receptors in Anopheles gambiae. Science 298: 176-178.
10. Nene V, Wortman J R, Lawson D, et al. (2007) Genome sequence of Aedes aegypti, a major arbovirus vector. Science 316: 1718-1723.
11. Hauser F, Cazzamali G, Williamson M, Blenau W, Grimmelikhuijzen J P (2006) A review of neurohormone GPCRs present in the fruitfly Drosophila melanogaster and the honey bee Apis mellifera. Prog Neurobiol 80:1-19.
12. Kebabian J W, Calne D B (1979) Multiple receptors for dopamine. Nature 277: 93-96.
13. Yellman C, Tao H, He B, Hirsh J (1997) Conserved and sexually dimorphic behavioral responses to biogenic amines in decapitated Drosophila. Proc Natl Acad Sci USA 94: 4131-4136.
14. Draper I, Kurshan P T, McBride E, Jackson F R, Kopin A S (2007) Locomotor activity is regulated by D2-like receptors in Drosophila: an anatomic and functional analysis. Dev Neurobiol 67: 378-393.
15. Mustard J A, Pham P M, Smith B H (2010) Modulation of motor behavior by dopamine and the D-1 like receptor AmDOP2 in the honey bee. J Insect Physiol 56: 422-430.
16. Kume K, Kume S, Park S K, Hirsh J, Jackson F R (2005) Dopamine is a regulator of arousal in the fruit fly. J Neurosci 25: 7377-7384.
17. Kim Y C, Lee H G, Han, K A (2007) D1 dopamine receptor dDA1 is required in the mushroom body neurons for aversive and appetitive learning in Drosophila. J Neurosci 27: 7640-7647.
18. Riemensperger T, Isabel G, Coulom H, Neuser K, Seugnet L, et al. (2011) Behavioral consequences of dopamine deficiency in the Drosophila central nervous system. Proc Natl Acad Sci USA 108: 834-839.
19. Ali D W (1997) The aminergic and peptidergic innervation of insect salivary glands. J Exp Biol 200: 1941-1949.
20. Sauer J R, Essenberg R C, Bowman A S (2000) Salivary glands in Ixodid ticks: control and mechanism of secretion. J Insect Physiol 46: 1069-1078.
21. Andersen J P, Schwartz A, Gramsbergen J B, Loeschcke V (2006) Dopamine levels in the mosquito Aedes aegypti during adult development, following blood feeding and in response to heat stress. J Insect Phys 52: 1163-1170.
22. Ferdig M T, Taft A S, Smartt C T, Lowenberger C A, Li J, et al. (2000) Aedes aegypti dopa decarboxylase: gene structure and regulation. Insect Mol Biol 9: 231-239.
23. Johnson J K, Li J, Christensen B M (2001) Cloning and characterization of a dopachrome conversion enzyme from the yellow fever mosquito, Aedes aegypti. Insect Biochem Mol Biol 31: 1125-1135.
24. Huang C—Y, Christensen B M, Chen C-C (2005a) Role of dopachrome conversion enzyme in the melanization of filarial worms in mosquitoes. Insect Mol Biol 14: 675-682.
25. Huang C—Y, Chou S—Y, Bartholomay L C, Christensen B M, Chen C-C (2005b) The use of gene silencing to study the role of dopa decarboxylase in mosquito melanization reactions. Insect Mol Biol 14: 237-244.
26. Hodgetts R B, O'Keefe S L (2006) Dopa decarboxylase: A model gene-enzyme system for studying development, behavior, and systematics Annu Rev Entomol 51: 259-284.
27. Moreira L A, Ye Y H, Turner K, Eyles D W, McGraw E A, et al. (2011) The wMelPop strain of Wolbachia interferes with dopamine levels in Aedes aegypti. Parasites and Vectors 4: 28.
28. Pratt S, Pryor S C (1986) Dopamine- and octopamine-sensitive adenylate cyclase in the brain of adult Culex pipiens mosquitoes. Cellular Mol Neurobiol 6: 325-329.
29. Lawson, D, Arensburger, P, Atkinson, P, Besansky, N J, Bruggner R V, et al. (2008) VectorBase: a data resource for invertebrate vector genomics. Nucleic Acids Res 37: Database issue, D583-587.
30. Gotzes F, Balfanz S, Baumann A (1994) Primary structure and functional characterization of a Drosophila dopamine receptor with high homology to human D1/5 receptors. Receptor Chan 2: 131-141.
31. Feng G, Hannan F, Reale V, Hon Y Y, Kousky C T, et al. (1996) Cloning and functional characterization of a novel dopamine receptor from Drosophila melanogaster. J Neurosci 16: 3925-3933.
32. Rutherford K, Parkhill J, Crook J, Horsnell T, Rice P, et al. (2000) Artemis: sequence visualization and annotation. Bioinformatics 16: 944-945.
33. Chema R, Sugawara H, Koike T, Lopez R, Gibson T J, et al. (2003) Multiple sequence alignment with the Clustal series of programs Nucleic Acids Res 31: 3497-3500.
34. Swofford D L (2001) Phylogenetic analysis using parsimony (*and other methods) Version 4. Sinauer Assoc, Sunderland, Mass.
35. Comet F (1988) Multiple sequence alignment with hierarchical clustering”. Nucleic Acids Res 16: 10881-10890.
36. Meyer J M, Ejendal K F K, Watts V J, Hill C A (2011) Molecular and pharmacological characterization of two D₁-like dopamine receptors in the Lyme disease vector, Ixodes scapularis. Insect Biochem Mol Biol 41: 563-571.
37. Przybyla J A, Cueva J P, Chemel B R, Hsu K J, Riese D J, et al. (2009) Comparison of the enantiomers of (±)-doxanthrine, a high efficacy full dopamine D₁ receptor agonist, and a reversal of enantioselectivity at D₁ versus alpha_{2c} adrenergic receptors. Eur Neuropsychopharmacol 19: 138-146.
38. Zhang J-H, Chung T D Y, Oldenburg K R (1999) Statistical parameter for use in evaluation and validation of high throughput screening assays. J Biomol Screen 4: 67-73.
39. Sugamori K S, Demchyshyn L L, McConkey F, Forte M A, Niznik H B (1995) A primordial dopamine D₁-like adenylyl cyclase-linked receptor from Drosophila melanogaster displaying poor affinity for benzazepines. FEBS Lett 362: 131-138.
40. Mustard J A, Blenau W, Hamilton I S, Ward V K, Ebert P R, et al. (2003) Analysis of two D₁-like dopamine receptors from the honey bee Apis mellifera reveals agonist-independent activity. Mol Brain Res 12: 67-77.
41. \check{S}imo L, Ko\check{c}i J, \check{Z}it\check{n}an D, Park Y (2011) Evidence for D1 dopamine receptor activation by a paracrine signal of dopamine in tick salivary glands. PLoS One 6: e16158.
42. Mustard J A, Beggs K T, Mercer A R (2005) Molecular biology of the invertebrate dopamine receptors. Arch Insect Biochem Physiol 59: 103-117.
43. Han K-A, Millar N S, Grotewiel M S, Davis R L (1996) DAMB, a novel dopamine receptor expressed specifically in Drosophila mushroom bodies. Neuron 16: 1127-1135.
44. Han K-A, Millar N S, Davis R L (1998) A novel octopamine receptor with preferential expression in Drosophila mushroom bodies. J Neurosci 18: 3650-3658.
45. Grohmann L, Blenau W, Erber J, Ebert P R, Strunker T, et al. (2003) Molecular and functional characterization of an octopamine receptor from honeybee (Apis mellifera) brain. J Neurochem 86: 725-735.
46. Lee D-W, Pietrantonio P V (2003) In vitro expression and pharmacology of the 5-HT₇-like receptor present in the mosquito Aedes aegypti tracheolar cells and hindgut-associated nerves. Insect Mol Biol 12: 561-569.
47. Belmont M, Cazzamali G, Williamson M, Hauser F, Grimmelikhuizen C J P (2006) Identification of four evolutionarily related G protein-coupled receptors from the malaria mosquito Anopheles gambiae. Biochem Biophys Res Comm 344: 160-165.
48. Hansen K K, Stafflinger E, Schneider M, Hauser F, Cazzamali G, et al. (2010) Discovery of a novel insect neuropeptide signaling system closely related to the insect adipokinetic hormone and corazonin hormonal systems. J Biol Chem 285: 10736-10747.
49. Kim Y—C, Lee H-G, Seong C—S, Han K-A (2003) Expression of a D1 dopamine receptor dDA1/DmDOP1 in the central nervous system of Drosophila melanogaster. Gene Exp Pat 3: 237-245.
50. Blenau W, Erber J, Baumann A (1998) Characterization of a dopamine receptor from Apis mellifera: Cloning, functional expression, pharmacology, and mRNA localization in the brain. J Neurochem 70: 15-23.
51. Kokay I C, Ebert P R, Kirchhof B S, Mercer A R (1999) Distribution of dopamine receptors and dopamine receptor homologs in the brain of the honey bee, Apis mellifera L. Microscopy Res Tech 44: 179-189.
52. Blenau W, Baumann A (2001) Molecular and pharmacological properties of insect biogenic amine receptors: Lessons from Drosophila melanogaster and Apis mellifera. Arch Insect Biochem Physiol 48: 13-38.
53. Humphries M A, Mustard J A, Hunter S J, Mercer A, Ward V, et al. (2003) Invertebrate D2 type dopamine receptor exhibits age-based plasticity of expression in the mushroom bodies of the honeybee brain. J Neurobiol 55: 315-330.
54. Strader C D, Sigal I S, Candelore M R, Rands E, Hill W S, et al. (1988) Conserved aspartic acid residue-79 and residue-113 of the beta-adrenergic receptor have different roles in receptor function. J Biol Chem 263: 10267-10271.
55. Strader C D, Candelore M R, Hill W S, Sigal I S, Dixon R A F (1989) Identification of two serine residues involved in agonist activation of the beta-adrenergic receptor. J Biol Chem 264: 13572-13578.
56. Pollock N J, Manelli A M, Hutchins C W, Steffey M E, MacKenzie R G, et al. (1992) Serine mutations in transmembrane-V of the dopamine D1-receptor affect ligand interactions and receptor activation. J Biol Chem 267: 17780-17786.
57. Dixon R A F, Sigal I S, Candelore M R, Register R B, Scattergood W, et al. (1987) Structural features required for ligand binding to the β-adrenergic receptor. EMBO J. 6: 3269-3275.
58. Fraser C M, Chung F Z, Wang C D, Venter J C (1988) Site-directed mutagenesis of human β-adrenergic receptors: substitution of aspartic acid-130 by asparagine produces a receptor with high-affinity agonist binding that is uncoupled from adenylate cyclase. Proc Natl Acad Sci USA 85: 5478-5482.
59. Karnik S S, Sakmar T P, Chen H B, Khorana H G (1988) Cysteine residues 110 and 187 are essential for the formation of the correct structure in bovine Rhodopsin. Proc Natl Acad Sci USA 85: 8459-8463.
60. Fraser C M (1989) Site-directed mutagenesis of (3-adrenergic receptors: identification of conserved cysteine residues that independently affect ligand binding and receptor activation. J Biol Chem 264: 9266-9270.
61. Reale V, Hannan F, Hall L M, Evans P D (1997) Agonist-specific coupling of a cloned Drosophila melanogaster D1-like dopamine receptor to multiple second messenger pathways by synthetic agonists. J Neurosci 17: 6545-6554.
62. Sanyal S, Wintle R F, Kindt K S, Nuttley W M, Aryan R, et al. (2004) Dopamine modulates the plasticity of mechanosensory responses in Caenorhabditis elegans. EMBO J. 23: 473-482.
63. Tiberi M, Caron M G (1994) High agonist-independent activity is a distinguishing feature of the dopamine D1B receptor subtype. J Biol Chem 269: 27925-27931.
64. Seifert R, Wenzel-Seifert K (2002) Constitutive activity of G protein-coupled receptors: cause of disease and common property of wild-type receptors. Naunyn-Schmiedeberg's Arch Pharmacol 366: 381-416.
65. Raymond-Delpech V, Matsuda K, Sattelle B, Rauh J J and Sattelle D B (2005) Ion channels: molecular targets of neuroactive insecticides. Invert Neurosci 5: 119-133.
66. Missale C, Nash S R, Robinson S W, Jaber M, Caron M G (1998) Dopamine receptors: from structure to function. Physiol Rev 78, 189-225.
67. Pridgeon J W, Becnel J J, Clark G G, Linthicum K J (2009) A high-throughput screening method to identify potential pesticides for mosquito control. J Med Entomol 46: 335-341.
68. Mugumbate G, Jackson G E, van der Spoel D (2010) Open conformation of adipokinetic hormone receptor from the malaria mosquito facilitates hormone binding. Peptides 32: 553-559.
69. Hirashima A, Eiraku T, Kuwano E, Eto M (2004) Comparative receptor surface analysis of agonists for tyramine receptor which inhibit sex-pheremone production in Plodia interpunctella. Comb Chem High Throughput Screen 8: 589-594.
70. Senderowitz H, Marantz Y (2009) G protein-coupled receptors: target-based in silico screening. Curr Pharm Des 15: 4049-4068.
71. Casida J E, Quistad G B (1998) Golden age of insecticide research: past, present or future? Annu Rev Entomol 43:1-16.
72. Benting J, Leonhardt M, Lindell S D, Tiebes J (2005) The design, synthesis and screening of a muscarinic acetylcholine receptor targeted compound library. Comb Chem High Throughput Screen 8: 649-653.
73. Roeder T (2005) Tyramine and octopamine: ruling behavior and metabolism. Annu Rev Entomol 50: 447-477.
74. Sunahara R K, Niznik H B, Weiner D M, Stormann T M, Brann M R, et al. (1990) Human dopamine D₁ receptor encoded by an intronless gene on chromosome 5. Nature 347: 80-83.
75. Sunahara R K, Guan H C, O'Dowd B F, Seeman P, Laurier L G, et al. (1991) Cloning of the gene for a human dopamine D₅ receptor with higher affinity for dopamine than D₁. Nature 350: 614-619.
76. Sunahara, R. K., Niznik, H. B., Weiner, D. M., Stormann, T. M., Brann, M. R., Kennedy, J. L., Gelernter, J. E., Rozmahel, R., Yang, Y., Israel, Y., Seeman, P., O'Dowd, B. F., 1990. Human dopamine D₁ receptor encoded by an intronless gene on chromosome 5. Nature 347, 80-83.
77. Bloomquist, 1996; Casida and Quistad, 1998
78. Ejendal, K. F. K., Meyer, J. M., Brust, T. F., Avramova. L., Garland-Kuntz, E., Giraldo-Calderon, G., Hill, C. A., and Watts, V. J. Discovery of antagonists of tick dopamine receptors via chemical library screening and comparative pharmacological analyses. Insect Biochem. Mol. Biol. In press.
79. Extermination, Insect and Pest Control Services in the US: 56171. IBISWorld Industry Report. Nov. 23, 2009.
80. Gondhalekar, A. D., Song, C. and Scharf, M. E. 2011. Development of strategies for monitoring indoxacarb and gel bait susceptibility in the German cockroach (Blattodea: Blattellidae). Pest. Manag. Sci. 67, 262-70.
81. Meyer, J. M., Ejendal, K. F. K., Avramova, L. V., Garland-Kuntz, E. E., Giraldo-Calderon, G. I., Brust, T. F., Watts, V. J and Hill, C. A. 2012. A “Genome-to-Lead” Approach for Insecticide Discovery: Pharmacological Characterization and Screening of Aedes aegypti D1-like Dopamine Receptors. PLoS Negl. Trop. Dis. 6; -1478.
82. WHO. 1998. Test Procedures for Insecticide Resistance Monitoring in Malaria Vectors, Bio-efficacy and Persistence of Insecticides on Treated Surfaces. Document WHO/CDS/CPC/MAL/98. 12. World Health Organization, Geneva.
83. Altschul, S. F., Madden, T. L., Schäffer, A. A., Zhang, J., Zhang, Z., Miller, W., Lipman, D. J., 1997. Gapped BLAST and PSI-BLAST: a new generation of protein database search programs. Nucleic Acids Res. 25, 3389-3402.
84. Blenau, W., Erber, J., Baumann, A., 1998. Characterization of a dopamine receptor from Apis mellifera: cloning, functional expression, pharmacology, and mRNA localization in the brain. J. Neurochem. 70, 15-23.
85. Blenau, W., Baumann, A., 2001. Molecular and pharmacological properties of insect biogenic amine receptors: lessons from Drosophila melanogaster and Apis mellifera. Arch. Insect Biochem. Physiol. 48, 13-38.
86. Bowman, A. S., Sauer, J. R., 2004. Tick salivary glands: function, physiology and future. Parasitology 129, S67-S81.
87. Caragea, C., Sinapov, J., Silvescu, A., Dobbs, D., Honavar, V., 2007. Glycosylation site prediction using ensembles of Support Vector Machine classifiers. BMC Bioinform. 8, 438.
88. Chen, A., Holmes, S. P., Pietrantonio, P. V., 2004. Molecular cloning and functional expression of a serotonin receptor from the Southern cattle tick, Boophilus microplus (Acari: Ixodidae). Insect Mol. Biol. 13, 45-54.
89. Chema, R., Sugawara, H., Koike, T., Lopez, R., Gibson, T. J., Higgins, D. G., Thompson, J. D., 2003. Multiple sequence alignment with the Clustal series of programs. Nucleic Acids Res. 31, 3497-3500
90. Civelli, O., Bunzow, J. R., Grandy, D. K., 1993. Molecular diversity of the dopamine receptors. Annu Rev. Pharmacol. Toxicol. 32, 281-307.
91. Corpet, F., 1988. Multiple sequence alignment with hierarchical clustering. Nucleic Acids Res. 16 (22), 10881-10890.
92. Dixon, R. A. F., Sigal, I. S., Candelore, M. R., Register, R. B., Scattergood, W., Rands, E., Strader, C. D., 1987. Structural features required for ligand binding to the badrenergic receptor. EMBO J. 6 (11), 3269-3275.
93. Feng, G., Hannan, F., Reale, V., Hon, Y. Y., Kousky, C. T., Evans, P. D., Hall, L. M., 1996. Cloning and functional characterization of a novel dopamine receptor from Drosophila melanogaster. J. Neurosci. 16, 3925-3933.
94. Filmore, D., 2004. It's a GPCR world. Mod. Drug Discov. 7 (11), 24e28. Fraser, C. M., Chung, F. Z., Wang, C. D., Venter, J. C., 1988. Site-directed mutagenesis of human b-adrenergic receptors: substitution of aspartic acid-130 by asparagine produces a receptor with high-affinity agonist binding that is uncoupled from adenylate cyclase. Proc. Natl. Acad. Sci. U.S.A. 85, 5478-5482.
95. Fraser, C. M., 1989. Site-directed mutagenesis of b-adrenergic receptors: identification of conserved cysteine residues that independently affect ligand binding and receptor activation. J. Biol. Chem. 264, 9266-9270.
96. Gether, U., 2000. Uncovering molecular mechanisms involved in activation of G protein-coupled receptors. Endocr. Rev. 21, 90-113.
97. Gotzes, F., Balfanz, S., Baumann, A., 1994. Primary structure and functional characterization of a Drosophila dopamine receptor with high homology to human D1/5 receptors. Recept. Channels 2, 131-141.
98. Grandy, D. K., Marchionni, M. A., Makam, H., Stofko, R. E., Alfano, M., Frothingham, L., Fischer, J. B., Burke-Howie, K. J., Bunzow, J. R., Server, A. C., Civelli, O., 1989. Cloning of the cDNA and gene for a human D2 dopamine receptor. Proc. Natl. Acad. Sci. U.S.A. 86, 9762-9766.
99. Grohmann, L., Blenau, W., Erber, J., Ebert, P. R., Strunker, T., Baumann, A., 2003. Molecular and functional characterization of an octopamine receptor from honeybee (Apis mellifera) brain. J. Neurochem. 86, 725-735.
100. Han, K.-A., Millar, N. S., Grotewiel, M. S., Davis, R. L., 1996.DAMB, a novel dopamine receptor expressed specifically in Drosophila mushroom bodies. Neuron 16, 1127-1135.
101. Han, K.-A., Millar, N. S., Davis, R. L., 1998. A novel octopamine receptor with preferential expression in Drosophila mushroom bodies. J. Neurosci. 18 (10), 3650-3658.
102. Hearn, M. G., Ren, Y., McBride, E. W., Reveillaud, I., Beinborn, M., Kopin, A. S., 2002. A Drosophila dopamine 2-like receptor: molecular characterization and identification of multiple alternatively spliced variants. Proc. Natl. Acad. Sci. U.S.A. 99, 14554-14559.
103. Holmes, S. P., Barhoumi, R., Nachman, R. J., Pietrantonio, P. V., 2003. Functional analysis of a G protein-coupled receptor from the Southern cattle tick Boophilus microplus (Acari: Ixodidae) identifies it as the first arthropod myokinin receptor. Insect Mol. Biol. 12, 27-38.
104. Humphries, M. A., Mustard, J. A., Hunter, S. J., Mercer, A., Ward, V., Ebert, P. R., 2003. Invertebrate D2 type dopamine receptor exhibits age-based plasticity of expression in the mushroom bodies of the honeybee brain. J. Neurobiol. 55, 215-330.
105. Jin, H., Xie, Z., George, S. R., O'Dowd, B. F., 1999. Palmitoylation occurs at cysteine 347 and cysteine 351 of the dopamine D(1) receptor. Eur. J. Pharmacol. 386, 305-312.
106. Jongejan, F., Uilenberg, G., 2004. The global importance of ticks. Parasitology 129, S3-S14.
107. Karnik, S. S., Sakmar, T. P., Chen, H. B., Khorana, H. G., 1988. Cysteine residues 110 and 187 are essential for the formation of the correct structure in bovine rhodopsin. Proc. Natl. Acad. Sci. U.S.A. 85, 8459-8463.
108. Karpa, K. D., Lidow, M. S., Pickering, M. T., Levenson, R., Bergson, C., 1999. N-linked glycosylation is required for plasma membrane localization of D5, but not D1, dopamine receptors in transfected mammalian cells. Mol. Pharmacol. 56, 1071-1078.
109. Kyte, J., Doolittle, R. F., 1982. A simple method for displaying the hydropathic character of a protein. J. Mol. Biol. 157, 105-132.
110. Lawson, D., Arensburger, P., Atkinson, P., et al., 2009. VectorBase: a data resource for invertebrate vector genomics. Nucleic Acids Res. 37, D583-D587. Database issue.
111. Lees, K., Bowman, A. S., 2007. Tick neurobiology: recent advances and the postgenomic era. Invert. Neurosci. 7, 183-198.
112. Meyer, J. M., Kurtti, T. J., Van Zee, J. P., Hill, C. A., 2010. Genome organization of major tandem repeats in the hard tick, Ixodes scapularis. Chrom. Res. 18, 357-370.
113. Missale, C., Nash, S. R., Robinson, S. W., Jaber, M., Caron, M. G., 1998. Dopamine receptors: from structure to function. Physiol. Rev. 78, 189-225.
114. Mustard, J. A., Blenau, W., Hamilton, I. S., Ward, V. K., Ebert, P. R., Mercer, A. R., 2003. Analysis of two D1-like dopamine receptors from the honey bee Apis mellifera reveals agonist-independent activity. Mol. Brain. Res. 12, 67-77.
115. Mustard, J. A., Beggs, K. T., Mercer, A. R., 2005. Molecular biology of the invertebrate dopamine receptors. Arch. Insect Biochem. Physiol. 59, 103-117.
116. Namkung, Y., Sibley, D. R., 2004. Protein kinase C mediates phosphorylation, desensitization, and trafficking of the D2 dopamine receptor. J. Biol. Chem. 279, 49533-49541.
117. Ohta, H., Tsuchihara, K., Mitsumasu, K., Yaginuma, T., Ozoe, Y., Asaoka, K., 2009. Comparative pharmacology of two D1-like dopamine receptors cloned from the silkworm Bombyx mori. Insect Biochem. Mol. Biol. 39, 342-347.
118. Palczewski, K., Kumasaka, T., Hori, T., Behnke, C. A., Motoshima, H., Fox, B. A., Trong, I. L., Teller, D. C., Okada, T., Stenkamp, R. E., Yamamoto, M., Miyano, M., 2000. Crystal structure of Rhodopsin: a G protein-coupled receptor. Science 289, 739-745.
119. Pollock, N. J., Manelli, A. M., Hutchins, C. W., Steffey, M. E., MacKenzie, R. G., Frail, D. E., 1992. Serine mutations in transmembrane-V of the dopamine D1-receptor affect ligand interactions and receptor activation. J. Biol. Chem. 267, 17780-17786.
120. Probst, W. C., Snyder, L. A., Schuster, D. I., Brosius, J., Sealfon, S. C., 1992. Sequence alignment of the G-protein coupled receptor superfamily. DNA Cell Biol. 11, 1-20.
121. Przybyla, J. A., Cueva, J. P., Chemel, B. R., Hsu, K. J., Riese, D. J., McCorvy, J. D., Chester, J. A., Nichols, D. E., Watts, V. J., 2009. Comparison of the enantiomers of (_)-doxanthrine, a high efficacy full dopamine D1 receptor agonist, and a reversal of enantioselectivity at D1 versus alpha2C adrenergic receptors. Eur. Neuropsychopharmacol. 19 (2), 138-146.
122. Reale, V., Hannan, F., Hall, L. M., Evans, P. D., 1997. Agonist-specific coupling of a cloned Drosophila melanogaster D1-like dopamine receptor to multiple second messenger pathways by synthetic agonists. J. Neurosci. 17, 6545-6554.
123. Ren, J., Wen, L., Gao, X., Jin, C., Xue, Y., Yao, X., 2008. CSS-Palm 2.0: an updated software for palmitoylation sites prediction. Prot. Engin. Des. Sel. 21, 639e644. Rutherford, K., Parkhill, J., Crook, J., Horsnell, T., Rice, P., Rajandream, M.-A., Barrell, B., 2000. Artemis: sequence visualization and annotation. Bioinformatics 16 (10), 944-945.
124. Sanyal, S., Wintle, R. F., Kindt, K. S., Nuttley, W. M., Aryan, R., Fitzmaurice, P., Bigras, E., Merz, D. C., Hebert, T. E., van der Kooy, D., Schafer, W. R., Culotti, J. G., Van Tol, H. H., 2004. Dopamine modulates the plasticity of mechanosensory responses in Caenorhabditis elegans. EMBO J. 23, 473-482.
125. Sauer, J. R., Essenberg, R. C., Bowman, A. S., 2000. Salivary glands in ixodid ticks: control and mechanism of secretion. J. Insect Physiol. 46, 1069-1078.
126. Seifert, R., Wenzel-Seifert, K., 2002. Constitutive activity of G protein-coupled receptors: cause of disease and common property of wild-type receptors. Naunyn-Schmiedeberg's Arch. Pharmacol. 366, 381-416.
127. Sillitoe, R. V., Vogel, M. W., 2008. Desire, disease, and the origins of the dopaminergic system. Schizophrenia Bull. 34, 212-219.
128. Simo, L., Ko_ci, J., _Zit_nan, D., Park, Y., 2011. Evidence for D1 dopamine receptor activation by a paracrine signal of dopamine in tick salivary glands. LOS One 6, -16158.
129. Sokoloff, P., Giros, B., Martres, M.-P., Bouthenet, M.-L., Schwartz, J.-C., 1990. Molecular cloning and characterization of a novel dopamine receptor (D3) as a target for neuroleptics. Nature 347, 146-151.
130. Strader, C. D., Sigal, I. S., Candelore, M. R., Rands, E., Hill, W. S., Dixon, R. A. F., 1988. Conserved aspartic acid residue-79 and residue-113 of the beta-adrenergic receptor have different roles in receptor function. J. Biol. Chem. 263, 10267-10271.
131. Strader, C. D., Candelore, M. R., Hill, W. S., Sigal, I. S., Dixon, R. A. F., 1989. Identification of two serine residues involved in agonist activation of the beta-adrenergic receptor. J. Biol. Chem. 264, 13572-13578.
132. Strader, C. D., Fong, T. M., Graziano, M. P., Tota, M. R., 1995. The family of G-proteincoupled receptors. FASEB J. 9, 745-754.
133. Sugamori, K. S., Demchyshyn, L. L., McConkey, F., Forte, M. A., Niznik, H. B., 1995. A primordial dopamine D1-like adenylyl cyclase-linked receptor from Drosophila melanogaster displaying poor affinity for benzazepines. FEBS Lett. 362, 131-138.
134. Sunahara, R. K., Niznik, H. B., Weiner, D. M., Stormann, T. M., Brann, M. R., Kennedy, J. L., Gelernter, J. E., Rozmahel, R., Yang, Y., Israel, Y., Seeman, P., O'Dowd, B. F., 1990. Human dopamine D1 receptor encoded by an intronless gene on chromosome 5. Nature 347, 80-83.
135. Sunahara, R. K., Guan, H.-C., O'Dowd, B. F., Seeman, P., Laurier, L. G., Ng, G., George, S. R., Torchia, J., Van Tol, H. H. M., Niznik, H. B., 1991. Cloning of the gene for a human dopamine D5 receptor with higher affinity for dopamine than D1. Nature 350, 614-619.
136. Swofford, D. L., 2001. Phylogenetic Analysis Using Parsimony (*And Other Methods) Version 4. Sinauer Assoc., Sunderland, Mass.
137. Tiberi, M., Caron, M. G., 1994. High agonist-independent activity is a distinguishing feature of the dopamine D1B receptor subtype. J. Biol. Chem. 269, 27925-27931.
138. Ullmann, A. J., Piesman, J., Dolan, M. C., Black, W. C. I. V., 2002. A preliminary linkage map of the tick, Ixodes scapularis. Exp. Appl. Acarol. 28, 107-126.
139. Van Tol, H. H. M., Bunzow, J. R., Guan, H.-C., Sunahara, R. K., Seeman, P., Niznik, H. B., Civelli, O., 1991. Cloning of the gene for a human dopamine D4 receptor with high affinity for the antipsychotic clozapine. Nature 350, 610-614.
140. Van Zee, J. P., Geraci, N. S., Guerrero, F. D., Wikel, S. K., Stuart, J. J., Nene, V. M., Hill, C. A., 2007. Tick genomics: the Ixodes genome project and beyond. Intl. J. Parasitol. 37, 1297-1305
141. Bacon, R. M., Kugeler, K. J., Mead, P. S., 2008. Surveillance for Lyme disease-United States, 1992e2006. MMWR Surveill. Summ. 57, 1-9.
142. Bai, H., Zhu, F., Shah, K., Palli, S. R., 2011. Large-scale RNAi screen of G proteincoupled receptors involved in larval growth, molting and metamorphosis in the red flour beetle. BMC Genom. 12, 388.
143. Blenau, W., Baumann, A., 2001. Molecular and pharmacological properties of insect biogenic amine receptors: lessons from Drosophila melanogaster and Apis mellifera. Arch. Insect Biochem. Physiol. 48, 13-38.
144. Bowman, A. S., Sauer, J. R., 2004. Tick salivary glands: function, physiology and future. Parasitology 129 (Suppl.), S67-S81.
145. Day, M. J., 2011. One health: the importance of companion animal vector-borne diseases. Parasit Vectors 4, 49.
146. Ejendal, K. F. K., Przybyla, J. A., Watts, V. J., 2011. Adenylyl cyclase isoform-specific signaling of GPCRs. In: Siehler, S., Milligan, G. (Eds.), G Protein-Coupled Receptors Structure, Signaling, and Physiology. Cambridge University Press, pp. 189-216.
147. Feng, G., Hannan, F., Reale, V., Hon, Y. Y., Kousky, C. T., Evans, P. D., Hall, L. M., 1996. Cloning and functional characterization of a novel dopamine receptor from Drosophila melanogaster. J. Neurosci. 16, 3925-3933.
148. Filmore, D., 2004. It's a GPCR world. Mod. Drug Discov. (7), 24-28.
149. George, J. E., Pound, J. M., Davey, R. B., 2004. Chemical control of ticks on cattle and the resistance of these parasites to acaricides. Parasitology 129 (Suppl.), S353-S366.
150. Gotzes, F., Balfanz, S., Baumann, A., 1994. Primary structure and functional characterization of a Drosophila dopamine receptor with high homology to human D1/5 receptors. Recept. Channels 2, 131-141.
151. Hall, T. A., 1999. BioEdit: a user-friendly biological sequence alignment editor and analysis program for Windows 95/98/NT. Nucl. Acids Symp. Ser. 41, 95-98.
152. Iversen, S. D., Iversen, L. L., 2007. Dopamine: 50 years in perspective. Trends Neurosci. 30, 188-193.
153. Iversen, P. W., Eastwood, B. J., Sittampalam, G. S., Cox, K. L., 2006. A comparison of assay performance measures in screening assays: signal window, Z′ factor, and assay variability ratio. J. Biomol. Screen. 11, 247-252.
154. Jongejan, F., Uilenberg, G., 2004. The global importance of ticks. Parasitology 129 (Suppl.), S3-14.
155. Meyer, J. M., Ejendal, K. F. K., Watts, V. J., Hill, C. A., 2011. Molecular and pharmacological characterization of two D(1)-like dopamine receptors in the Lyme disease vector, Ixodes scapularis. Insect Biochem. Mol. Biol. 41, 563-571.
156. Meyer, J. M., Ejendal, K. F., Avramova, L. V., Garland-Kuntz, E. E., Giraldo-Calderon, G. I., Brust, T. F., Watts, V. J., Hill, C. A., 2012. A “genome-to-lead” approach for insecticide discovery: pharmacological characterization and screening of Aedes aegypti D(1)-like dopamine receptors. PLoS Negl Trop. Dis. 6. -1478.
157. Mustard, J. A., Blenau, W., Hamilton, I. S., Ward, V. K., Ebert, P. R., Mercer, A. R., 2003. Analysis of two D1-like dopamine receptors from the honey bee Apis mellifera reveals agonist-independent activity. Brain Res. Mol. Brain. Res. 113, 67-77.
158. Mustard, J. A., Beggs, K. T., Mercer, A. R., 2005. Molecular biology of the invertebrate dopamine receptors. Arch. Insect Biochem. Physiol. 59, 103-117.
159. Overington, J. P., Al-Lazikani, B., Hopkins, A. L., 2006. How many drug targets are there? Nat. Rev. Drug Discov. 5, 993-996.
160. Riemensperger, T., Isabel, G., Coulom, H., Neuser, K., Seugnet, L., Kume, K., Iche-Torres, M., Cassar, M., Strauss, R., Preat, T., Hirsh, J., Birman, S., 2011. Behavioral consequences of dopamine deficiency in the Drosophila central nervous system. Proc. Natl. Acad. Sci. USA 108, 834-839.
161. Rosario-Cruz, R., Almazan, C., Miller, R. J., Dominguez-Garcia, D. I., Hernandez-Ortiz, R., de la Fuente, J., 2009. Genetic basis and impact of tick acaricide resistance. Front. Biosci. 14, 2657-2665.
162. Simo, L., Ko_ci, J., _Zit_nan, D., Park, Y., 2011. Evidence for D1 dopamine receptor activation by a paracrine signal of dopamine in tick salivary glands. PLoS One 6, -16158.
163. Suarez, C. E., Noh, S., 2011. Emerging perspectives in the research of bovine babesiosis and anaplasmosis. Vet. Parasitol. 180, 109-125.
164. Sunahara, R. K., Niznik, H. B., Weiner, D. M., Stormann, T. M., Brann, M. R., Kennedy, J. L., Gelernter, J. E., Rozmahel, R., Yang, Y. L., Israel, Y., et al., 1990. Human dopamine D1 receptor encoded by an intronless gene on chromosome 5. Nature 347, 80-83.
165. Sunahara, R. K., Guan, H. C., O'Dowd, B. F., Seeman, P., Laurier, L. G., Ng, G., George, S. R., Torchia, J., Van Tol, H. H., Niznik, H. B., 1991. Cloning of the gene for a human dopamine D5 receptor with higher affinity for dopamine than D1. Nature 350, 614-619.
166. Zhang, J. H., Chung, T. D., Oldenburg, K. R., 1999. A simple statistical parameter for use in evaluation and validation of high throughput screening assays. J. Biomol. Screen. 4, 67-73.

While the novel technology has been illustrated and described in detail in the figures and foregoing description, the same is to be considered as illustrative and not restrictive in character, it being understood that only the preferred embodiments have been shown and described and that all changes and modifications that come within the spirit of the novel technology are desired to be protected. As well, while the novel technology was illustrated using specific examples, theoretical arguments, accounts, and illustrations, these illustrations and the accompanying discussion should by no means be interpreted as limiting the technology. All patents, patent applications, and references to texts, scientific treatises, publications, and the like referenced in this application are incorporated herein by reference in their entirety.

1-41. (canceled)

42. A method of controlling a population of invertebrates, comprising the steps of:
contacting an invertebrate with a compound that includes at least one of the group consisting of asenapine, amperozide, cis-(Z)-flupenthixol, benztropine, methoiothepin, loxapine, mianserin, clominpramine, amperozide, butaclamol, clozapine, doxepin, and SCH23390 amitriptyline, chlorpromazine chlorprothixene, (±)-butaclamol hydrochloride, doxepin hydrochloride, cis-(Z)-flupenthixol dihydrochloride, methiothepin maleate, mianserin hydrochloride, niclosamide, piceatannol, and resveratrol.

43. The method of claim 42, wherein the invertebrate is at least one of an insect, a tick, a termite, a mosquito, or a cockroach.

44. The method of claim 42, further comprising the steps of:
contacting the invertebrate with a compound that includes at least one of the group of chemical scaffolds consisting of dibenzocycloheptane derivatives, phenothiazine derivatives, thioxanthene derivatives, butyrophenone derivatives, diphenyl amine-containing compounds, quinazoline derivatives, benzodiazoxide derivatives, indole derivatives, piperzinylpyrazolopyrimidines, aryldiaminopryimidine, and hexahydrothienopyridines.

45. The method of claim 42, further comprising the steps of:
contacting the invertebrate with a compound that includes at least 3 conjugated or non-conjugated rings, wherein said rings are independently selected from the group consisting of aromatic, non-aromatic, heterocyclic and non-heterocyclic rings, and wherein heterocyclic rings may independently include at least one of the following atoms: C, O, N, or S and wherein each ring may be independently, substituted or unsubstituted with at least one of the following groups or atoms including H, amines, imines, ketones, aldehydes, alcohols, thiols, aromatic rings, alkanes, alkenes, alkynes, halogens and the like.

46. The method of claim 42, further comprising the steps of:
contacting the insect with at least one polynucleotide that interferes with the expression of a gene having at least 90 percent homology to at least one of SEQ ID. NO. 1 or SEQ ID. NO. 3.

47. The method according to claim 46, wherein the polynucleotide interferes with the expression of at least one gene that hybridizes under stringent conditions to SEQ ID. NO. 1.

48. The method according to claim 46, wherein the polynucleotide interferes with the expression of at least one gene that includes SEQ ID. NO. 1.

49. The method according to claim 46, wherein the polynucleotide interferes with the expression of at least one gene that hybridizes under stringent conditions to SEQ ID. NO. 3.

50. The method according to claim 46, wherein the polynucleotide interferes with the expression of at least one gene that includes SEQ ID. NO. 3.

51. A method for screening, comprising the steps of:
expressing a polynucleotide having at least 90 percent homology to at least one of SEQ ID. NO. 1 and SEQ ID. NO. 3; and
contacting cells that express said polynucleotide with at least one exogenous compound, wherein said polynucleotide is not expressed in its native host.

52. The method according to claim 51, wherein said polynucleotide has at least 90 percent identity to at least one of SEQ ID. NO. 1 and SEQ ID. NO. 3.

53. The method according to claim 51, wherein said polynucleotide has at least 95 percent identity to SEQ ID. NO. 1.

54. The method according to claim 51, wherein said polynucleotide is SEQ ID. NO. 1.

55. The method according to claim 51, wherein said polynucleotide has at least 95 percent homology to SEQ ID. NO. 3.

56. The method according to claim 51, wherein said polynucleotide has at least 90 percent identity to SEQ ID. NO. 3.

57. The method according to claim 51, wherein said polynucleotide is SEQ ID. NO. 3.

58. A method for screening, further comprising the step of:
contacting an isolated polypeptide having at least 90 percent homology to at least one of SEQ ID. NO. 2 and SEQ ID. NO. 4; with at least one exogenous compound; and
detecting an interaction between said polypeptide and the at least one compound.

59. The method according to claim 58, wherein said polypeptide has at least 90 percent identity to SEQ ID. NO. 2.

60. The method of claim 58, further comprising the step of:
detecting an interaction between said polypeptide and the compound.

61. The method according to claim 58, wherein said polypeptide has at least 90 percent identity to SEQ ID. NO. 4.


SEQUENCE LISTING (8 sequences)

SEQ ID NO: 1 - DNA, 1239 bp, Aedes aegypti
atgaatacgattgggattttttggcgatattggtttctctcgagagccctcacaaacccactccaacacaagcaaacaacacaaccgaattctaaccgacgccccgtcttttcttctgcttccctttccgttgcaggtgtttttctatcgattgtgatatttctctccgtagccggtaatattctagtgtgtatagcaatctacaccgagcggagtctgcggcggattgggaatctcttcctggcgtcgcttgccatcgccgatctgttcgtggcatcgttggtgatgacgttcgccggtgtcaacgatttactgggctactggatattcggggcccagttctgcgacacctgggtggcgttcgatgtcatgtgttcaaccgcttccatcctgaatctgtgcgccatctcgctggaccggtacatccacatcaaggatccgctcagatatggccgctgggtaacgcgccgggttgccatcgggacgatcgtcgtcatctggctgctggcggccctggtctcgtttgtgccgatctcgctggacctgcaccgggacaagcgggacgaaacggacacctcgctcatcatcaacggcgtcaagtacgaaacgtgcgccctggacctgacgcccacctatgccgtcgtttctagttgtataagtttctacgtaccttgtatagttatgatcgggatctactgcagactctactgctacgcccagaagcacgtcaaatcgatacgagcggtgacgaggccgggtgaaatcagcgagaagcgctaccgcagcatacggaggcccaaaccgagcaaaaacaagctgaaattgcggcagctggcccatcacgcctcttcgccgtaccacgtgtcggaccacaaggcggccatcaccgtcggagtgatcatgggagtgtttctcgtctgctgggtgccgttcttctgtgtaaatatcatcgccgcgttctgcaagacctgtatcgggccgcagacgttcaaggtgctgtcctggctgggctactctaactcggccttcaatccgattatctactcgatcttcaataccgagttccgggaggcgttcaagcggatcctgacgacgaggagctcgtggtgctgtgcgcaggagatgggcaacattcatccgcggcacagcgaccgctacgtgacggactacgcggccaaaaatgtggtcatgaactccgggcgatcgtccgccgatctcgaacaggtatccgccatctga

SEQ ID NO: 2 - protein, 412 aa, Aedes aegypti
MNTIGIFWRYWFLSRALTNPLQHKQTTQPNSNRRPVFSSASLSVAGVFLSIVIFLSVAGNILVCIAIYTERSLRRIGNLFLASLAIADLFVASLVMTFAGVNDLLGYWIFGAQFCDTWVAFDVMCSTASILNLCAISLDRYIHIKDPLRYGRWVTRRVAIGTIVVIWLLAALVSFVPISLDLHRDKRDETDTSLIINGVKYETCALDLTPTYAVVSSCISFYVPCIVMIGIYCRLYCYAQKHVKSIRAVTRPGEISEKRYRSIRRPKPSKNKLKLRQLAHHASSPYHVSDHKAAITVGVIMGVFLVCWVPFFCVNIIAAFCKTCIGPQTFKVLSWLGYSNSAFNPIIYSIFNTEFREAFKRILTTRSSWCCAQEMGNIHPRHSDRYVTDYAAKNVVMNSGRSSADLEQVSAI

SEQ ID NO: 3 - DNA, 1431 bp, Aedes aegypti
atgaataatgcaactgacttctactgggaactgtcgcaactgataaggtttagcaatctcagctactacaatgtgtcaaatggtggcgaaacttcggtgacactggatgcggtcaactgctgtaccactgagccgaacttctcagagtacctggatgccctgccaaacgacagggccggattgttagcgtttctgttcctgttttccttcgcaacagtcttcggtaactcgctggtaatactggctgttatccgagagcggtaccttcacacggcgaccaattatttcgtgaccagcctggccgtggcggactgcctggtaggactggtcgtgatgccgttctctgcattgtatgaggtgctgcagaatacgtggttcttcggtgaagattggtgtgatatatggcgctcgttagatgtattgtttagtactgcttccatactgaacctatgtgtgatatctcttgatcggtactgggccataacggatccattttcctacccgatgaagatgactcgaaagaaggcagctgccctaatagcagctgtgtggatatgctcaagtgcgatcagcttcccggcgatcgtgtggtggcgagctgcacgggaaggcgatatgccaccgtacaagtgcacatttaccgagcatctcggctatttggtgttctcctccaccatatcgttctatcttccactgcttgtaatggtgttcacttattgtagaatataccgagctgcagcaatacaaactcgatcgctcaagctgggaaccaagcaagtgctgatggcatccggtgaactacaacttaccctccgaatacatcgaggtggcaccactcgagaaaggcccaaccatcagcaacagcaaccgccgcagcaccaaacgcatcagcatcaagaccatcagcagcacacggcgaattcgacaccggaagaaccggacgaggaaccgctttccgcgctgcagaacaatggccttggagggcgccatcggacgcacatgggcaagaacttttccctgtcgcgaaagctggccaagtttgccaaggagaagaaggccgccaaaacgctcggcattgtgatgggtgtgttcatagtttgttggcttccattttttgtggtgaacctgctgtcggggttttgtatgcagtgtatagcgcacgaggaaattgtgtcggccgtggtcacgtggctggggtggatcaattccagtatgaatccggtgatctacgcgtgttggagcagagatttcagaagggccttcgtgcggatattgtgtgtttgctgccccagaaagatccggcgaaaataccagccaacaatgcgctcgaaagcgtctcagagatttgcatccagacggtgttactcgacgtgttccctgcatgggatccagcaagtacgtcaaaactcttgcgagcagacgtatatctag

SEQ ID NO: 4 - protein, 476 aa, Aedes aegypti
MNNATDFYWELSQLIRFSNLSYYNVSNGGETSVTLDAVNCCTTEPNFSEYLDALPNDRAGLLAFLFLFSFATVFGNSLVILAVIRERYLHTATNYFVTSLAVADCLVGLVVMPFSALYEVLQNTWFFGEDWCDIWRSLDVLFSTASILNLCVISLDRYWAITDPFSYPMKMTRKKAAALIAAVWICSSAISFPAIVWWRAAREGDMPPYKCTFTEHLGYLVFSSTISFYLPLLVMVFTYCRIYRAAAIQTRSLKLGTKQVLMASGELQLTLRIHRGGTTRERPNHQQQQPPQHQTHQHQDHQQHTANSTPEEPDEEPLSALQNNGLGGRHRTHMGKNFSLSRKLAKFAKEKKAAKTLGIVMGVFIVCWLPFFVVNLLSGFCMQCIAHEEIVSAVVTWLGWINSSMNPVIYACWSRDFRRAFVRILCVCCPRKIRRKYQPTMRSKASQRFASRRCYSTCSLHGIQQVRQNSCEQTYI

SEQ ID NO: 5 - DNA, 1278 bp, Ixodes scapularis
atgcagccttcttcgtccagcaccctgggttccattgcgagcgtactacccaccttggaggcagcggccaacaacaccaccgggatcggtcaggacggccaggtgtcctccggccagctggacagttgggacctgtcctgccgctacatcatcggcaccttcctctcactcatcatcctgacggccatcaccggcaacattcttgtctgcctggccatctacacggatcgccggctccgcaagctgggcaacctcttcctcgtttctctggccgtggccgacttgtttgtgtcctcactggtgatgacgttcgcggtggttaacgacctcatgggatactgggcgttcggcccccagttctgcgacatctggattgccttcgatgtgatgtgcagcacggcgtccattctgaacttgtgcgccatcagcatggaccgttttctgcacatcaaggacccgctgggctacggccgctggatgaccaagcgtgccgtgctgggcaccatctgtgccatctggatgttgtcagcgttgatgtcgtttctgcccatctcgctgggctggcaccggccgtatccggactcactgcttctggtcaacggactcacaatgtgtgcgctggacttgacgccggagtacgcggtcacatcgtctctcattagcttctacatgccctgcgtggtcatggtggcgttgtacacgcggctctacctgtacgcgcgcaagcacgtgcagaacatccgcgccgtcaccaagcccatgaaccacaaagacatgagccccaccaagttccgcagcatgggacagtcctcgctgcacgtgatggaccacaaagccgccatcacgctcggaatcatcgtgggtgtgttcctctgctgctgggtgcccttcttctgcgccaacattgtggccgccttctgcaagacctgcatctccgaagattgcttcaagtttctcacctggctgggctacctcaactcggcgctcaacccaatcatctacagcatcttcaacacggagtttagggacgccttccgcagggtcatcacggctcacgcatgctgcaaggcgctcgccgagagggcgagggcacccagcgacctctcctatcgcaccaagagaagcaccatttgcgagacgcccagggcgaacagtgtggccaattccgcgggatgccgaagcaacaacaacactgccgaacgcatttctctccgcgccgaagtcgacacccgggtcgagatcagcgtgaagagcgtgggcgagatctcggccatatag

SEQ ID NO: 6 - protein, 425 aa, Ixodes scapularis
MQPSSSSTLGSIASVLPTLEAAANNTTGIGQDGQVSSGQLDSWDLSCRYIIGTFLSLIILTAITGNILVCLAIYTDRRLRKLGNLFLVSLAVADLFVSSLVMTFAVVNDLMGYWAFGPQFCDIWIAFDVMCSTASILNLCAISMDRFLHIKDPLGYGRWMTKRAVLGTICAIWMLSALMSFLPISLGWHRPYPDSLLLVNGLTMCALDLTPEYAVTSSLISFYMPCVVMVALYTRLYLYARKHVQNIRAVTKPMNHKDMSPTKFRSMGQSSLHVMDHKAAITLGIIVGVFLCCWVPFFCANIVAAFCKTCISEDCFKFLTWLGYLNSALNPIIYSIFNTEFRDAFRRVITAHACCKALAERARAPSDLSYRTKRSTICETPRANSVANSAGCRSNNNTAERISLRAEVDTRVEISVKSVGEISAI

SEQ ID NO: 7 - DNA, 1374 bp, Ixodes scapularis
atgccagttccgggcgagacgaacggctcgctggacgagccggcgtacagcctgctggaactggtgtatggcgcgaacctgtccgtgctcgagctggagatcgtgcgcaacgacagcgtggtggacatcgtgcgcgaggcggcggttaacgtctcgctgtccttcgtggacgacgccggcaacgcgagcgggtccccgtttccggacgagtcgacgcaggggcagcacgtggcgctcggcgtactgctgggcttcttctgcttcgtgaccatcttcggcaatgtgctggtcatgctgtccgtggcgaaagagcagtacctgcacacggtgaccaactacttcatcgcgtcgctggcgacggcggactgcctggtgggcgccgtggtcatgcctttctcggccatccacgagatcatggacaagtactggatattcggccaggatctgtgcgacgtgtggcactcgatcgacgtgctcgccagtacggcgtccatcctcaacctctgcgtcatatcgctggatcgctattgggctatcactgatcccatcagctacccgtgccgtatgacgcaggccaaggcgaccaccctgatcgccgtcgtctgggtctgctccgcgctcatctccttcccggccatcgcgtggtggcgcgccgtcaacaagggtccgccggccgccttccagtgcgccttcaccgacgacgtgggctacctggtcttctcgtccaccatctccttctacgccccgctcatggtcatggtgttcacctactaccgcatctaccgagccgccgtcgaacagacgcggaacctcaagctcggctcaaagcaagtacagtcctgcaacggcgacgagaccacttcggtcacgctgcgaatccaccgcgggggaatgctgcaggcagcgaacaactacaagacagtcttttcgacctcgaacgagtccttcgaccggaacaaccttacgggggtgtccaggaacctcaaaaacttttcgctcagccgaaagctcgccaagctcgccaaggaacgcaaggccgccaagacgctcgccatcgtcatgggcgtgttcatcctgtgctggctgcccttcttcgtcaccaatatcctcatgggcatctgcggcgagaactgcgtcatgcagccggacctcgtattctccacagtgacctggctcggctggatcaactccggcatgaaccccatcatctacgcctgctggagcagggacttccgacgagctttcgccaacgtgctctgctgctgctgcccgggctacttccgccgacgacagcggcgaagagaccgcctccgacgcatcatcaaggaagacgcgagcatgcggtcgcagagcctagaggaggccgtcctatag

SEQ ID NO: 8 - protein, 457 aa, Ixodes scapularis
MPVPGETNGSLDEPAYSLLELVYGANLSVLELEIVRNDSVVDIVREAAVNVSLSFVDDAGNASGSPFPDESTQGQHVALGVLLGFFCFVTIFGNVLVMLSVAKEQYLHTVTNYFIASLATADCLVGAVVMPFSAIHEIMDKYWIFGQDLCDVWHSIDVLASTASILNLCVISLDRYWAITDPISYPCRMTQAKATTLIAVVWVCSALISFPAIAWWRAVNKGPPAAFQCAFTDDVGYLVFSSTISFYAPLMVMVFTYYRIYRAAVEQTRNLKLGSKQVQSCNGDETTSVTLRIHRGGMLQAANNYKTVFSTSNESFDRNNLTGVSRNLKNFSLSRKLAKLAKERKAAKTLAIVMGVFILCWLPFFVTNILMGICGENCVMQPDLVFSTVTWLGWINSGMNPIIYACWSRDFRRAFANVLCCCCPGYFRRRQRRRDRLRRIIKEDASMRSQSLEEAVL